(19) United States
(12) Patent Application Publication
(10) Pub. No.: US 2025/0009748 A1
(43) Pub. Date: Jan. 9, 2025

(54) COMBINATION THERAPY USING SUBSTITUTED PYRIMIDIN-4(3H)-ONES AND SOTORASIB

(71) Applicants: NAVIRE PHARMA, INC., Palo Alto, CA (US); BRIDGEBIO SERVICES, INC., Palo Alto, CA (US)

(72) Inventors: Pedro BELTRAN, Palo Alto, CA (US); Carl DAMBKOWSKI, Palo Alto, CA (US); Justin LIM, Palo Alto, CA (US); Anna WADE, Palo Alto, CA (US); Eli WALLACE, Palo Alto, CA (US); Yuting SUN, Palo Alto, CA (US); Nancy KOHL, Palo Alto, CA (US); Brooke MEYERS, Palo Alto, CA (US); Kerstin SINKEVICIUS, Palo Alto, CA (US); James STICE, Palo Alto, CA (US); David VAN VEENHUYZEN, Palo Alto, CA (US); Lauren WOOD, Palo Alto, CA (US)

(21) Appl. No.: 18/696,931

(22) PCT Filed: Sep. 30, 2022
(86) PCT No.: PCT/US2022/045413

§ 371 Date: Mar. 28, 2024

Related U.S. Application Data
(60) Provisional application No. 63/250,883, filed on Sep. 30, 2021.

(51) Int. Cl. A61K 31/513; A61K 31/519; A61P 35/00

(52) U.S. Cl. CPC A61K 31/513; A61K 31/519; A61P 35/00

(57) ABSTRACT

The present disclosure provides a method of treating cancer in a subject. The method including administering to the subject: a) a therapeutically effective amount of a compound of formula (I); and b) a therapeutically effective amount of sotorasib, wherein the compound of formula (I) is represented by (I), or a pharmaceutically acceptable salt, hydrate, solvate, stereoisomer, conformational isomer, tautomer, or a combination thereof. In particular, the present disclosure provides a method of treating a solid tumor (e.g., an advanced or metastatic non-small cell lung cancer) with a therapeutically effective amount of a compound of formula (10b) (i.e., 6-((3S,4S)-4-amino-3-methyl-2-oxa-8-azaspiro[4.5]decan-8-yl)-3-(Rₐ)-(2,3-dichlorophenyl)-2,5-dimethylpyrimidin-4(3H)-one) in combination with sotorasib in a subject, wherein the subject has one or more mutations in KRAS, such as KRAS G12C.

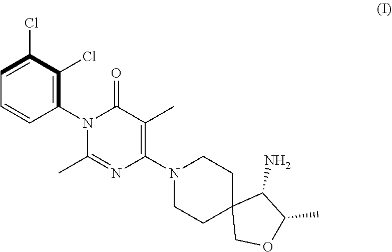

### CROSS-REFERENCES TO RELATED APPLICATIONS

This application claims priority to U.S. Provisional Application No. 63/250,883 filed Sep. 30, 2021, which is incorporated herein in its entirety for all purposes.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND

The mitogen-activated protein kinase (MAPK) signaling pathway plays critical roles in the regulation of diverse cellular activities, including cell proliferation, survival, differentiation, and motility. The MAPK signal transduction pathway is mediated by receptor tyrosine kinases (RTKs). Dysregulation of the MAPK pathway occurs in more than one-third of all malignancies. The classical MAPK pathway consists of Ras (a family of related proteins which is expressed in all animal cell lineages and organs), Raf (a family of three serine/threonine-specific protein kinases that are related to retroviral oncogenes), MEK (mitogen-activated protein kinase kinase), and ERK (extracellular signal-regulated kinases), sequentially relaying proliferative signals generated at the cell surface receptors into the nucleus through cytoplasmic signaling. RTKs and components of the MAPK pathway, such as RAS and RAF, are frequently activated by mutation in human cancers, resulting in constitutive pathway activation. Several RTK and MAPK pathway inhibitors, including epidermal growth factor receptor (EGFR), anaplastic lymphoma kinase (ALK), and MAPK inhibitors, have been approved for the treatment of solid tumors in which activation of these pathways is the oncogenic driver.

KRAS (Kirsten rat sarcoma 2 viral oncogene homolog) is a part of the RAS/MAPK pathway. KRAS is one of the most prevalent oncogenes in a variety of human cancers. Although there is compelling evidence that oncogenic KRAS drives tumorigenesis, efforts to target mutant KRAS have been stalled for many years. Recently, progress has been made in the development of therapeutics against cancers characterized by KRAS having a G12C mutation, which is present in ˜15% of lung adenocarcinoma and 0%-8% of other cancers. Results from early-stage clinical trials indicate that many cancer patients in this subgroup could significantly benefit from these novel therapeutics. The first KRAS G12C inhibitor, sotorasib, received accelerated approval by the Food and Drug Administration (FDA) for the treatment of adult patients with KRAS G12C-mutated locally advanced or metastatic NSCLC, as determined by an FDA-approved test, who have received at least one prior systemic therapy. Another KRAS G12C inhibitor, adagrasib, has been granted FDA breakthrough therapy designation for the same indication. Additional KRAS G12C inhibitors are also under investigation.

Despite progress with inhibitors of RTK and MAPK pathway inhibitors, resistance to these pathway inhibitors have been observed in both clinical and nonclinical studies. Such resistance may be driven by activation or upregulation of other components of the pathway. Accordingly, monotherapies comprising RTK and MAPK pathway inhibitors may not be sufficient for treating tumors that have developed or may develop resistance to approved pathway inhibitors.

Protein-tyrosine phosphatase non-receptor type 11 (PTPN11, also known as Src Homology-2 phosphatase (SHP2)) is a non-receptor protein tyrosine phosphatase encoded by the PTPN11 gene. SHP2 plays a key role in the RTK-mediated MAPK signal transduction pathway. This PTP contains two tandem Src homology-2 (SH2) domains, which function as phospho-tyrosine binding domains, a catalytic domain, and a C-terminal tail. In the basal state the protein typically exists in an inactive, self-inhibited conformation with the N-terminal SH2 domain blocking the active site. When stimulated by signal transduction mediated by cytokines and growth factor binding of phosphorylated proteins to the SH2 domains the auto-inhibition is relieved, this makes the active site available for dephosphorylation of PTPN11 substrates (MG Mohl, B G Neel, Curr. Opin. Genetics Dev. 2007, 17, 23-30. K S Grossmann, Adv. Cancer Res. 2010, 106, 53-89. W. Q. Huang et. al. Curr. Cancer Drug Targets 2014, 14, 567-588. C. Gordon et. al. Cancer Metastasis Rev. 2008, 27, 179-192.).

Germ-line and somatic mutations in PTPN11 have been reported in several human diseases resulting in gain-of-function in the catalytic activity, including Noonan Syndrome and Leopard Syndrome; as well as multiple cancers such as juvenile myelomonocytic leukemia, neuroblastoma, myelodysplastic syndrome, B cell acute lymphoblastic leukemia/lymphoma, melanoma, acute myeloid leukemia and cancers of the breast, lung and colon (MG Mohl, BG Neel, Curr. Opin. Genetics Dev. 2007, 17, 23-30). Recent studies have demonstrated that single PTPN11 mutations are able to induce Noonan syndrome, JMML-like myeloproliferative disease and acute leukemia in mice. These mutations disrupt the auto-inhibition between the N-SH2 domains and the catalytic site allowing constitutive access of substrates to the catalytic site of the enzyme (E. Darian et al, Proteins, 2011, 79, 1573-1588. Z-H Yu et al, JBC, 2013, 288, 10472, W Qiu et al BMC Struct. Biol. 2014, 14, 10).

PTPN11 is widely expressed in most tissues and plays a regulatory role in various cell signaling events that are important for a diversity of cell functions that includes proliferation, differentiation, cell cycle maintenance, epithelial-mesenchymal transition (EMT), mitogenic activation, metabolic control, transcription regulation, and cell migration, through multiple signaling pathways including the Ras-MAPK, the JAK-STAT or the PI3K-AKT pathways (Tajan, M. et. al. Eur. J. Medical Genetics, 2015, 58, 509-525. Prahallad, A. et. al. Cell Reports, 2015, 12, 1978-1985).

Additionally there is growing evidence that PTPN11/SHP2 is implicated in immune evasion during tumorigenesis, and hence a SHP2 inhibitor could stimulate the immune response in cancer patients (Cancer Res. 2015 Feb. 1; 75(3):508-18. T Yokosuka T, J Exp Med. 2012, 209(6), 1201. S Amamath Sci Transl Med. 2011, 3, 111ra120. T Okazaki, PNAS 2001, 98:24, 13866-71).

Substituted pyrimin-4(3H)-one compounds refer to a class of compounds having inhibitory activities against PTPN11/SHP2, as disclosed in International Patent Application No. PCT/US2019/045903 filed Aug. 9, 2019, and represented by the following formula:

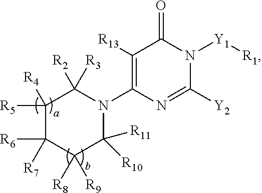

or a pharmaceutically acceptable salt, hydrate, solvate, stereoisomer, conformational isomer, tautomer, or a combination thereof, wherein the subscripts a and b, Y₁, Y₂, R₁, R₂, R₃, R₄, R₅, R₆, R₇, R₈, R₉, R₁₀, R₁₁, and R₁₃ are as provided in PCT/US2019/045903, which is incorporated herein in its entirety for all purposes. In particular, the substituted pyrimin-4(3H)-one compound is represented by formula (I):

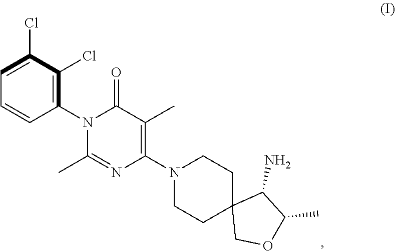

or a pharmaceutically acceptable salt, hydrate, solvate, stereoisomer, conformational isomer, tautomer, or a combination thereof. In certain embodiments, the compound of formula (I) is Compound (10b), represented by the formula:

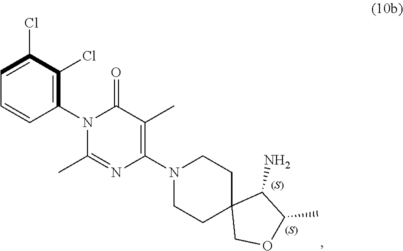

having the name of 6-((3S,4S)-4-amino-3-methyl-2-oxa-8-azaspiro[4.5]decan-8-yl)-3-(Rₐ)-(2,3-dichlorophenyl)-2,5-dimethylpyrimidin-4(3H)-one.

The compound of formula (I), in particular formula (10b), is a potent, selective, orally active allosteric inhibitor of Src Homology-2 Phosphatase (SHP2) (also known as protein tyrosine phosphatase non-receptor type 11 (PTPN11)), a tyrosine phosphatase that plays a key role in the receptor tyrosine kinase (RTK)-mediated mitogen activated protein kinase (MAPK) signal transduction pathway (Matozaki, 2009). Key components of the MAPK pathway include the small GTPase RAS, the serine/threonine-protein kinase RAF, mitogen-activated protein kinase (MEK), and extracellular signal activated kinase (ERK). In cells, SHP2 binds to phosphorylated tyrosine residues in the intracellular domain of RTKs such as the Epidermal Growth Factor Receptor (EGFR), leading to activation of the downstream MAPK signaling pathway.

As cancers may have or develop resistance to RTK and MAPK pathway inhibitors including KRAS G12C inhibitors, there remains a need for effective and safe therapeutic agents, including agents that may be used in combination, to treat cancers.

### SUMMARY

The present disclosure provides methods for treating various cancers, the methods including administration of a KRAS G12C inhibitor (e.g., sotorasib, also known as AMG 510) and a PTPN11 inhibitor (e.g., a compound of formula (I) or (10b)). The combination therapies provided herein are useful in the treatment of cancers including lung cancers (e.g., non-small cell lung cancer).

In one aspect, the present disclosure provides a method of treating cancer (e.g., an advanced or metastatic non-small cell lung cancer) in a subject in need thereof, the method including administering to the subject:
a) a therapeutically effective amount of a compound represented by formula (I):

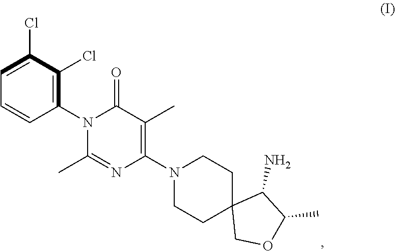

or a pharmaceutically acceptable salt, hydrate, solvate, stereoisomer, conformational isomer, tautomer, or a combination thereof; and
b) a therapeutically effective amount of sotorasib.

In another aspect, the present disclosure provides a pharmaceutical composition for treating cancer in a subject, the composition including:
a) a therapeutically effective amount of a compound represented by formula (I); and
b) a therapeutically effective amount of sotorasib, together with a pharmaceutically acceptable carrier or excipient,
   wherein the compound of formula (I) is as defined and described herein.

In another aspect, the present disclosure provides a kit for treating cancer in a subject, the kit including:
a) a therapeutically effective amount of a compound represented by formula (I); and
b) a therapeutically effective amount of sotorasib,
   together with instruction for effective administration,
      wherein the compound of formula (I) is as defined and described herein.

In some embodiments, the compound of formula (I) is represented by formula (10b):

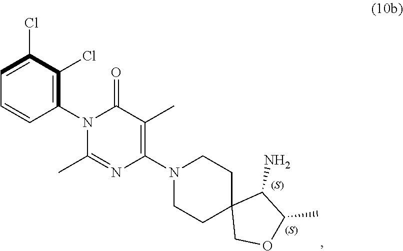

having the name of 6-((3S,4S)-4-amino-3-methyl-2-oxa-8-azaspiro[4.5]decan-8-yl)-3-(Rₐ)-(2,3-dichlorophenyl)-2,5-dimethylpyrimidin-4(3H)-one.

### BRIEF DESCRIPTION OF THE DRAWINGS

FIGS. 1A-1E show the compound of formula (10b) in combination with Compound A (i.e., 6-fluoro-7-(2-fluoro-6-hydroxyphenyl)-1-[4-methyl-2-(propan-2-yl)pyridin-3-yl]-4-[(2S)-2-methyl-4-(prop-2-enoyl)piperazin-1-yl]pyrido[2,3-d]pyrimidin-2(1H)-one) suppresses the growth of NCI-H358 subcutaneous tumors at tolerated doses in a mouse model. FIGS. 1A, 1B, and 1D show tumor volume monitored bi-weekly by caliper. FIGS. 1C and 1E show body weights recorded. Data represent mean±SEM. N=8 mice/group for FIGS. 1B-1E and N=10 mice/group for FIG. 1A.

FIGS. 2A and 2B show changes in tumor volume and body weight in mice bearing NCI-H358 subcutaneous cell line-derived tumors upon treatment with compound of formula (10b) alone or in combination with sotorasib (AMG 510).

FIGS. 3A and 3B show changes in tumor volume and body weight in mice bearing NCI-H2122 subcutaneous cell line-derived tumors upon treatment with compound of formula (10b) alone or in combination with sotorasib (AMG 510).

FIGS. 4A-4D show in vitro viability data for the compound of formula (10b) and sotorasib (AMG 510) in an NCI-H358 (KRAS^{G12C}) model. FIG. 4A: 3D viability at various concentrations of formula (10b). FIG. 4B: Bliss energy scoring for combinations of AMG 510 and formula (10b). FIG. 4C: fold change in DUSP6 levels. FIG. 4D: fold change in SPRY4 levels. As shown in FIGS. 4C-4D, the combination of AMG 510 and formula (10b) suppressed DUSP6 and SPRY4 levels relative to either agent alone.

FIG. 5 shows a Phase 1A/1B study of formula (10b) in combination with sotorasib in patients with solid tumors with a KRAS G12C mutation. The study design includes Phase 1a dose escalation and Phase 1b dose expansion/optimization.

FIG. 6 shows a flowchart for a trial conducted using the BOIN Design. Abbreviations: BOIN=Bayesian optimal interval design; DLT=dose limiting toxicity; MTD=maximum tolerated dose. Note: λe=19.7% and λd=29.8%. In practice, with 6 patients/cohort, if the DLT rate is ≤1/6 then escalate the dose, if the DLT rate is ≥2/6 then de-escalate the dose.

### DETAILED DESCRIPTION

### I. General

The present disclosure provides a combination therapy method of cancer (e.g., a solid tumor) in a subject. The method includes administering to the subject a) a therapeutically effective amount of a compound of formula (I) (as a PTPN11 inhibitor); and b) a therapeutically effective amount of sotorasib, wherein the compound of formula (I) is as defined and described herein. In particular, the cancer is characterized by a KRAS mutation, such as a mutation other than a Q61X mutation, e.g., a KRAS G12C mutation. In some instances, the cancer is a solid tumor, such as an advanced or metastatic non-small cell lung cancer (NSCLC). Also provided are a pharmaceutical composition thereof and a kit thereof for treating cancer in a subject.

### II. Definitions

As used herein, the terms below have the meanings indicated.

“Comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

“About,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that figure as well, taking into account significant figures.

“Salt” refers to acid or base salts of the compounds of the present disclosure. Illustrative examples of pharmaceutically acceptable acid addition salts are mineral acid (hydrochloric acid, hydrobromic acid, phosphoric acid, and the like) salts and organic acid (acetic acid, propionic acid, glutamic acid, citric acid and the like) salts. Examples of pharmaceutically acceptable base addition salts include sodium, potassium, calcium, ammonium, organic amino, or magnesium salt, or a similar salt. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“Solvate” refers to a compound provided herein or a salt thereof, that further includes a stoichiometric or non-stoichiometric amount of solvent bound by non-covalent intermolecular forces.

“Hydrate” refers to a compound that is complexed to a water molecule. The compounds of the present disclosure can be complexed with 1% water molecule or from 1 to 10 water molecules.

Asymmetric centers exist in the compounds disclosed herein. These centers are designated by the symbols “R” or “S,” depending on the configuration of substituents around the chiral carbon atom. It should be understood that the present disclosure encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method. Starting compounds of particular stereochemistry are either commercially available or can be made and resolved by various techniques. Additionally, the compounds disclosed herein may exist as geometric isomers. The present disclosure includes all cis, trans, syn, anti, entgegen (E), and zusammen (Z) isomers as well as the appropriate mixtures thereof. Additionally, compounds may exist as tautomers; all tautomeric isomers are provided by this present disclosure. Additionally, the compounds disclosed herein can exist in unsolvated as well as solvated forms with pharmaceutically acceptable solvents such as water, ethanol, and the like. In general, the solvated forms are considered equivalent to the unsolvated forms.

“Tautomer”, as use herein, alone or in combination, refers to one of two or more isomers that rapidly interconvert. Generally, this interconversion is sufficiently fast so that an individual tautomer is not isolated in the absence of another tautomer. The ratio of the amount of tautomers can be dependent on solvent composition, ionic strength, and pH, as well as other solution parameters. The ratio of the amount of tautomers can be different in a particular solution and in the microenvironment of a biomolecular binding site in said solution. Examples of tautomers include keto/enol, enamine/imine, and lactam/lactim tautomers. Additional examples of tautomers also include 2-hydroxypyridine/2(1H)-pyridone and 2-aminopyridine/2(1H)-iminopyridone tautomers.

Conformational isomers exist in the compounds disclosed herein. When R₁ is aryl or heteroaryl in the formula:

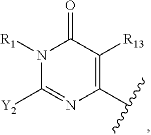

the aryl or heteroaryl group can orient in different conformations in relation to the pyrimidinone moiety, as represented by:

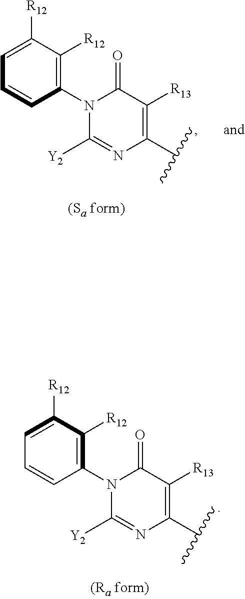

These forms are designated by the symbols “Sₐ” or “R_{d}”, depending on the conformation of the aryl or heteroaryl group in relation to the pyrimidinone moiety. Examples of “Sₐ” and “Rₐ” forms can be found in Examples 1-20 of International Patent Application No. PCT/US2019/045903, which is incorporated herein in its entirety for all purposes. The compound of formula (10b) is substantially in a “Rₐ” form.

“Pharmaceutically acceptable” refers to those compounds (salts, hydrates, solvates, stereoisomers, conformational isomers, tautomers, etc.) which are suitable for use in contact with the tissues of patients without undue toxicity, irritation, and allergic response, are commensurate with a reasonable benefit/risk ratio, and are effective for their intended use. The compounds disclosed herein can exist as pharmaceutically acceptable salts, as defined and described herein.

“Combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“PTPN11 inhibitor” is used herein to refer to a compound that exhibits an IC₅₀ with respect to PTPN11 activity of no more than about 100 micromolar (μM) and more typically not more than about 50 μM, as measured in the PTPN11 assay described generally in International Patent Application No. PCT/US2019/045903 (e.g., the enzymatic activity of recombinant human PTPN11 proteins of Example 21). “IC₅₀” is that concentration of inhibitor which reduces the activity of an enzyme (e.g., PTPN11) to half-maximal level. In certain embodiments, compounds disclosed in PCT/US2019/045903 exhibit an IC₅₀ of no more than about 10 μM for inhibition of PTPN11; in further embodiments, compounds exhibit an IC₅₀ of no more than about 1 μM for inhibition of PTPN11; in yet further embodiments, compounds exhibit an IC₅₀ of not more than about 200 nM for inhibition of PTPN11; in yet further embodiments, compounds exhibit an IC₅₀ of not more than about 100 nM for inhibition of PTPN11; and in yet further embodiments, compounds exhibit an IC₅₀ of not more than about 50 nM for inhibition of PTPN11, as measured in the PTPN11 assay described therein. In certain embodiments, the compound of formula (I) or (10b) exhibits an IC₅₀ of no more than 50 nM for inhibition of PTPN11 (e.g., a PTPN11-E76K mutant enzyme).

“Therapeutically effective amount” refers to an amount of a compound or of a pharmaceutical composition useful for treating or ameliorating an identified disease or condition, or for exhibiting a detectable therapeutic or inhibitory effect. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Treat”, “treating”, and “treatment” refer to any indicia of success in the treatment or amelioration of an injury, pathology, or condition, including any objective or subjective parameter such as abatement; remission; diminishing of symptoms or making the injury, pathology or condition more tolerable to the patient; slowing in the rate of degeneration or decline; making the final point of degeneration less debilitating; and/or improving a patient's physical or mental well-being. The treatment or amelioration of symptoms can be based on objective or subjective parameters; including the results of a physical examination, neuropsychiatric exams, and/or a psychiatric evaluation.

“Administering” refers to therapeutic provision of the compound or a form thereof to a subject, such as by oral administration or intravenous administration.

“Patient” or “subject” refers to a living organism suffering from or prone to a disease or condition that can be treated by administration of a pharmaceutical composition as provided herein. Non-limiting examples include humans, non-human primates (e.g., monkeys), goats, pigs, sheep, cows, deer, horses, bovines, rats, mice, rabbits, hamsters, guinea pigs, cats, dogs, and other non-mammalian animals. In some embodiments, the subject is human. In some embodiments, a subject is an adult (e.g., at least 18 years of age).

“Composition,” as used herein, is intended to encompass a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

“Pharmaceutically acceptable excipient” refers to a substance that aids the administration of an active agent to and absorption by a subject. Pharmaceutical excipients useful in the present disclosure include, but are not limited to, binders, fillers, disintegrants, lubricants, coatings, sweeteners, flavors and colors. Other pharmaceutical excipients can be useful in the present disclosure.

“Tablet” refers to solid pharmaceutical formulations with and without a coating. The term “tablet” also refers to tablets having one, two, three or even more layers, wherein each of the before mentioned types of tablets may be without or with one or more coatings. In some embodiments, tablets of the present disclosure can be prepared by roller compaction or other suitable means known in the art. The term “tablet” also comprises mini, melt, chewable, effervescent, and orally disintegrating tablets. Tablets include the compound of formula (I) or (10b) and one or more pharmaceutical excipients (e.g., fillers, binders, glidants, disintegrants, surfactants, binders, lubricants, and the like). Optionally, a coating agent can be also included. For the purposes of calculating percent weight of the tablet formulation, the amount of coating agent is not included in the calculation. That is, the percent weights reported herein are of the uncoated tablet.

Unless specifically indicated otherwise, the content of the compound of formula (I) or (10b) in, e.g., a tablet formulation is calculated based on the normalized weight of the compound of formula (I) or (10b) on a salt-free and anhydrous basis. That is, the salt and/or water content in the compound of formula (I) or (10b) is not included in the calculation.

“KRAS G12C inhibitor” as used herein refers to a compound which targets, decreases, or inhibits the synthesis or biological activity of KRAS (Kirsten rat sarcoma 2 viral oncogene homolog) by selectively modifying mutant cysteine 12 in G12C mutated KRAS. The KRAS G12C inhibitor may at least partially inhibit KRAS G12C. The KRAS G12C inhibitor may be a selective KRAS G12C inhibitor (e.g., having greater selectivity for KRAS having a G12C mutation over KRAS having another mutation such as a G12D mutation). In those cases, the selective KRAS G12C inhibitor may have high potency for KRAS G12C, along with low affinity for other KRAS mutations. The KRAS G12C inhibitor may be a covalent inhibitor (e.g., capable of covalently modifying cysteine 12). The KRAS G12C inhibitor may be a noncolvanet inhibitor. The KRAS G12C inhibitor may bind to an inactive (“GDP”) form of KRAS. The KRAS G12C inhibitor may bind to an active (“GTP”) form of KRAS. The KRAS G12C inhibitor may bind to both inactive (“GDP”) and active (“GTP”) forms of KRAS. Examples of KRAS G12C inhibitors include sotorasib (AMG 510), adagrasib (MRTX-849), MRTX1257, ARS-853, ARS-1620, JNJ-74699157 (ARS-3248), JDQ443, GDC-6036, JAB-21822, BI 1823911, MK-1084, LY3537982, and LY3499446.

“KRAS-positive cancer” refers to a cancer with the KRAS gene rearranged, mutated, or amplified. “KRAS G12C-positive cancer” refers to a cancer with the KRAS G12C gene rearranged, mutated, or amplified.

“A cancer resistant to a KRAS inhibitor” and/or “a cancer that is a KRAS-positive cancer resistant to a KRAS inhibitor” refer to a cancer or tumor that either fails to respond favorably to treatment with a prior KRAS inhibitor, or alternatively, recurs or relapses after responding favorably to a KRAS inhibitor. “A cancer resistant to a KRAS G12C inhibitor” and/or “a cancer that is a KRAS G12C-positive cancer resistant to a KRAS G12C inhibitor” refer to a cancer or tumor that either fails to respond favorably to treatment with a prior KRAS G12C inhibitor, or alternatively, recurs or relapses after responding favorably to a KRAS G12C inhibitor.

“Jointly therapeutically effective amount” as used herein means the amount at which the therapeutic agents, when given separately (in a chronologically staggered manner, especially a sequence-specific manner) to a warm-blooded animal, especially to a human to be treated, show an (additive, but preferably synergistic) interaction (joint therapeutic effect). Whether this is the case can be determined inter alia by following the blood levels, showing that both compounds are present in the blood of the human to be treated at least during certain time intervals.

“Synergistic effect” as used herein refers to an effect of at least two therapeutic agents: a PTPN11 inhibitor as defined herein; and a KRAS G12C inhibitor as defined herein, which is greater than the simple addition of the effects of each drug administered by themselves. The effect can be, for example, slowing the symptomatic progression of a proliferative disease, such as cancer, particularly lung cancer (e.g., non-small cell lung cancer), or symptoms thereof. Analogously, a “synergistically effective amount” refers to the amount needed to obtain a synergistic effect.

“A,” “an,” or “a(n)”, when used in reference to a group of substituents or “substituent group” herein, mean at least one. For example, where a compound is substituted with “an” alkyl or aryl, the compound is substituted with at least one alkyl and/or at least one aryl, wherein each alkyl and/or aryl is optionally different. In another example, where a compound is substituted with “a” substituent group, the compound is substituted with at least one substituent group, wherein each substituent group is optionally different.

### III. Combination Therapy

In one aspect, the present disclosure provides a method of treating cancer in a subject. The method includes administering to the subject:
a) a therapeutically effective amount of a compound represented by formula (I):

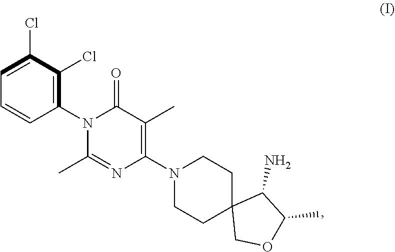

or a pharmaceutically acceptable salt, hydrate, solvate, stereoisomer, conformational isomer, tautomer, or a combination thereof; and
b) a therapeutically effective amount of sotorasib (AMG 510).

### III-1: Compound of Formula (I)

The compound of formula (I) can be in a pharmaceutically acceptable salt form or in a neutral form, each of which is optionally in a solvate or a hydrate form.

In some embodiments, the compound of formula (I) is in a pharmaceutically acceptable salt form. In some embodiments, a pharmaceutically acceptable acid addition salt of the compound of formula (I) is represented by formula (Ia):

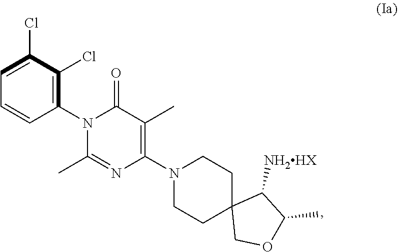

wherein HX is a pharmaceutically acceptable acid addition.

Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like.

In some embodiments, the compound of formula (I) is in a neutral form.

In some embodiments, the compound of formula (I) has a substantially moiety of 6-((3S,4S)-4-amino-3-methyl-2-oxa-8-azaspiro[4.5]decan-8-yl) with stereochemistry as shown in formula (10b):

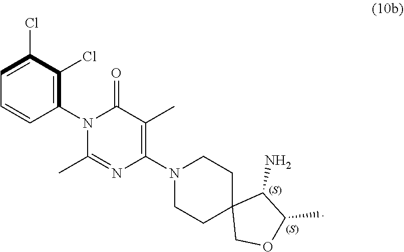

In some embodiments, the compound of formula (I) is substantially in a Rₐ conformation as shown in formula (10b):

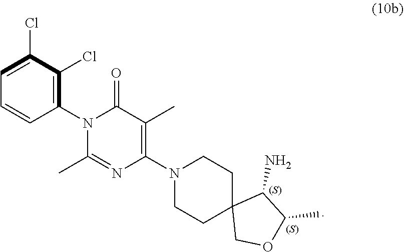

In some embodiments, the compound of formula (I) is represented by formula (10b):

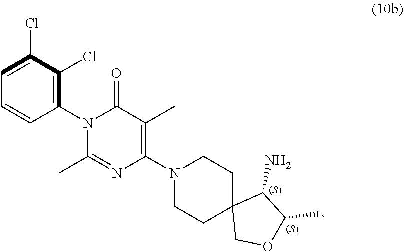

having the name of 6-((3S,4S)-4-amino-3-methyl-2-oxa-8-azaspiro[4.5]decan-8-yl)-3-(Ra)-(2,3-dichlorophenyl)-2,5-dimethylpyrimidin-4(3H)-one.

In some embodiments, the compound of formula (I) or (10b) is in a neutral form.

In some embodiments, the compound of formula (I) includes one or more corresponding enantiomer, diastereomers, and/or conformational isomers of the compound of formula (10b), as represented by formulae, respectively:

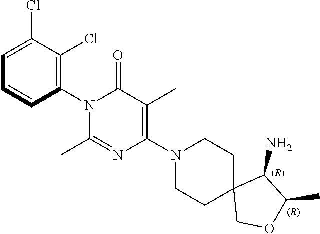

enantiomer (3R, 4R, Sₐ)

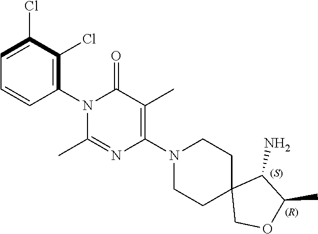

diastereomer (3R, 4S, Rₐ)

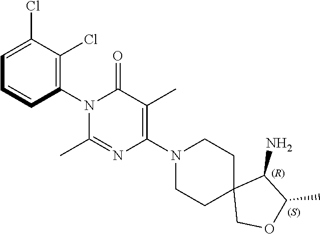

diastereomer (3S, 4R, Sₐ)

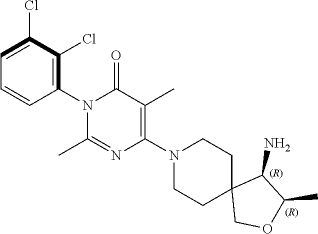

diastereomer (3R, 4R, Rₐ)

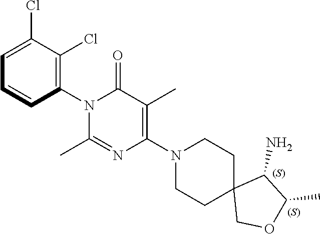

diastereomer (3S, 4S, Sₐ)

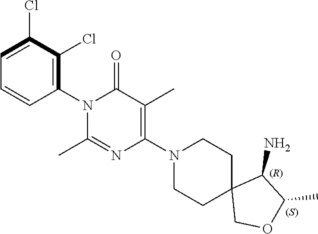

diastereomer (3S, 4R, Rₐ)

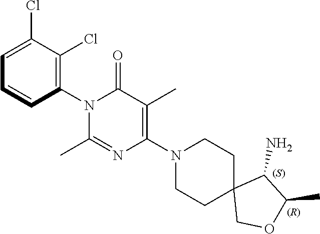

diastereomer (3R, 4S, Sₐ)

In some embodiments, the compound of formula (10b) has a purity of at least about 95 area % determined by a chiral high-performance liquid chromatography (HPLC). In some embodiments, the compound of formula (10b) has a purity of from about 95 area % to about 99 area %, from about 96 area % to about 99 area %, from about 97 area % to about 99 area %, or from about 98 area % to about 99 area %, determined by a chiral high-performance liquid chromatography (HPLC). In some embodiments, the compound of formula (10b) has a purity of from about 98 area % to about 99 area %.

In some embodiments, the compound of formula (I) includes one or more corresponding enantiomer, diastereomers, and/or conformational isomers of the compound of formula (10b), as represented by the formulae above; and a total of the one or more isomers is no more than about 5 area % determined by a chiral high-performance liquid chromatography (HPLC).

In some embodiments, the corresponding enantiomer, diastereomers, and/or conformational isomers of the compound of formula (10b) are present in the compound of formula (I) meet acceptance criteria as follows: enantiomer (3R, 4R, Sₐ)≤0.5 area %; diastereomer (3R, 4S, Rₐ)≤1.2 area %; diastereomer (3S, 4R, Sₐ)≤0.5 area %; diastereomer (3R, 4R, Rₐ)≤0.5 area %; diastereomer (3S, 4S, Sₐ)≤0.5 area %; diastereomer (3S, 4R, Rₐ)≤0.5 area %; and diastereomer (3R, 4S, Sₐ)≤0.5 area %, each of which is determined by a chiral high-performance liquid chromatography (HPLC). In some embodiments, the compound of formula (10b) has a purity of at least about 95 area %, wherein enantiomer (3R, 4R, Sₐ)<0.5 area %; diastereomer (3R, 4S, Rₐ)<1.2 area %; diastereomer (3S, 4R, Sₐ)<0.5 area %; diastereomer (3R, 4R, Rₐ)<0.5 area %; diastereomer (3S, 4S, Sₐ)<0.5 area %; diastereomer (3S, 4R, Rₐ)<0.5 area %; and diastereomer (3R, 4S, Sₐ)<0.5 area %, each of which is determined by a chiral high-performance liquid chromatography (HPLC). In some embodiments, the compound of formula (10b) has a purity of from about 95 area % to about 99 area %, from about 96 area % to about 99 area %, from about 97 area % to about 99 area %, or from about 98 area % to about 99 area %, wherein enantiomer (3R, 4R, Sₐ)<0.5 area %; diastereomer (3R, 4S, Rₐ)<1.2 area %; diastereomer (3S, 4R, Sₐ)<0.5 area %; diastereomer (3R, 4R, Rₐ)<0.5 area %; diastereomer (3S, 4S, Sₐ)<0.5 area %; diastereomer (3S, 4R, Rₐ)<0.5 area %; and diastereomer (3R, 4S, Sₐ)<0.5 area %, each of which is determined by a chiral high-performance liquid chromatography (HPLC). In some embodiments, the compound of formula (10b) has a purity of from about 98 area % to about 99 area %, wherein enantiomer (3R, 4R, Sₐ) is not detected; diastereomer (3R, 4S, Rₐ) is about 0.86 area %; diastereomer (3S, 4R, Sₐ) is not detected; diastereomer (3R, 4R, Rₐ) is about 0.07 area %; diastereomer (3S, 4S, Sₐ) is not detected; diastereomer (3S, 4R, Rₐ) is not detected; and diastereomer (3R, 4S, Sₐ) is not detected, each of which is determined by a chiral high-performance liquid chromatography (HPLC).

In some embodiments, the compound of any one of formulae (I), (Ia), and (10b) is in a solvate and/or a hydrate form.

### III-2. Cancer/Solid Tumor

The cancer can be any cancer that responds to the treatment of a PTPN11 inhibitor and/or a KRAS G12C inhibitor (e.g., sotorasib). In some embodiments, the cancer is caused and/or characterized by a KRAS mutation, such as a KRAS G12C mutation. In some embodiments, the cancer is characterized by a KRAS mutation other than a Q61X mutation. In some embodiments, the cancer is a KRAS-positive cancer. In some embodiments, the cancer is a KRAS G12C-positive cancer (e.g., a cancer characterized by a G12C mutation in KRAS).

The cancer can be characterized by a solid tumor and/or a liquid tumor. In some embodiments, the cancer includes a solid tumor. In some embodiments, the cancer includes a liquid tumor.

In some embodiments, the cancer is lung cancer, colorectal cancer, pancreatic cancer, urothelial carcinoma, stomach cancer, mesothelioma, or a combination thereof. In some embodiments, the cancer is non-small cell lung cancer (NSCLC). In some embodiments, the cancer is NSCLC characterized by a KRAS mutation, such as a KRAS G12C mutation. In certain embodiments, a KRAS protein includes a G12C mutation. In certain embodiments, the cancer is NSCLC characterized by a G12C mutation in KRAS. In some embodiments, the cancer is NSCLC characterized by a mutation in an epidermal growth factor receptor (EGFR) protein. In some embodiments, the cancer is NSCLC that is not characterized by a mutation in EGFR or anaplastic lymphoma kinase (ALK).

In some embodiments, the cancer is a KRAS G12C-positive cancer (e.g., a cancer characterized by a G12C mutation in KRAS). In some embodiments, the KRAS G12C-positive cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma. In some embodiments, the cancer is small bowel cancer, appendiceal cancer, endometrial cancer, hepatobiliary cancer, small cell lung cancer, cervical cancer, germ cell tumor, ovarian cancer, gastrointestinal neuroendocrine tumor, bladder cancer, myelodysplastic/myeloproliferative neoplasms, head and neck cancer, esophagogastric cancer, soft tissue sarcoma, mesothelioma, thyroid cancer, leukemia, or melanoma. In some embodiments, the KRAS G12C-positive cancer is non-small cell lung cancer, colorectal cancer, pancreatic cancer, appendiceal cancer, endometrial cancer, cancer of unknown primary, ampullary cancer, gastric cancer, small bowel cancer, sinonasal cancer, bile duct cancer, or melanoma. In some embodiments, the cancer is an advanced or metastatic KRAS G12C-positive solid tumor (e.g., lung cancer, colorectal cancer, pancreatic cancer, urothelial carcinoma, stomach cancer, mesothelioma, or a combination thereof). In some embodiments, the cancer is an advanced or metastatic KRAS G12C-positive non-small cell lung cancer (NSCLC). In some embodiments, the cancer is an advanced or metastatic KRAS G12C-positive solid tumor, provided that the solid tumor is other than non-small cell lung cancer (NSCLC).

The cancer can also be any cancer that is resistant to the treatment of a KRAS G12C inhibitor. In some embodiments, the cancer is resistant to a KRAS G12C inhibitor as defined and described herein. In some embodiments, the cancer is resistant to a KRAS G12C inhibitor that is an inhibitor of the inactive (“GDP”) form of KRAS. In some embodiments, the cancer is resistant to a KRAS G12C inhibitor that is an inhibitor of the active (“GTP”) form of KRAS. In some embodiments, the cancer is resistant to a KRAS G12C inhibitor that is an inhibitor of both the inactive (“GDP”) and active (“GTP”) forms of KRAS.

In some embodiments, the cancer is characterized by intrinsic and/or acquired resistance to a KRAS G12C inhibitor as defined and described herein. In some embodiments, the cancer is a KRAS G12C-positive cancer resistant to a KRAS G12C inhibitor as defined and described herein. In some embodiments, the cancer is a KRAS G12C-positive cancer characterized by intrinsic and/or acquired resistance to a KRAS G12C inhibitor as defined and described herein.

In some embodiments, the cancer is characterized by intrinsic and/or acquired resistance to another therapy such as a KRAS modulator, platinum-based therapy, or taxane therapy. In some embodiments, the cancer is characterized by intrinsic and/or acquired resistance to a KRAS G12C inhibitor. In some embodiments, the cancer is characterized by intrinsic and/or acquired resistance to a KRAS G12C inhibitor selected from the group consisting of sotorasib (AMG 510), adagrasib (MRTX-849), MRTX1257, ARS-853, ARS-1620, JNJ-74699157 (ARS-3248), JDQ443, GDC-6036, JAB-21822, BI 1823911, MK-1084, LY3537982, and LY3499446. In some embodiments, the cancer is characterized by intrinsic and/or acquired resistance to sotorasib (AMG 510). In some embodiments, the cancer is characterized by intrinsic and/or acquired resistance to adagrasib (MRTX-849).

In some embodiments, the cancer is resistant to a KRAS G12C inhibitor selected from the group consisting of sotorasib (AMG 510), adagrasib (MRTX-849), MRTX1257, ARS-853, ARS-1620, JNJ-74699157 (ARS-3248), JDQ443, GDC-6036, JAB-21822, BI 1823911, MK-1084, LY3537982, and LY3499446. In some embodiments, the cancer is resistant to sotorasib (AMG 510) or adagrasib (MRTX-849). In some embodiments, the cancer is resistant to sotorasib (AMG 510). In some embodiments, the cancer is resistant to adagrasib (MRTX-849). In some embodiments, the cancer is a KRAS G12C-positive cancer resistant to a KRAS G12C inhibitor selected from the group consisting of sotorasib (AMG 510), adagrasib (MRTX-849), MRTX1257, ARS-853, ARS-1620, JNJ-74699157 (ARS-3248), JDQ443, GDC-6036, JAB-21822, BI 1823911, MK-1084, LY3537982, and LY3499446. In some embodiments, the cancer is a KRAS G12C-positive cancer resistant to sotorasib (AMG 510) or adagrasib (MRTX-849). In some embodiments, the cancer is a KRAS G12C-positive cancer resistant to sotorasib (AMG 510). In some embodiments, the cancer is a KRAS G12C-positive cancer resistant to adagrasib (MRTX-849).

The solid tumor can be any solid tumor that responds to the treatment of a PTPN11 inhibitor and a KRAS G12C inhibitor (e.g., sotorasib). In some embodiments, the solid tumor is a tumor with one or more genes in KRAS rearranged, mutated, or amplified, provided that the tumor is other than caused by one or more additional activating mutations in BRAF V600X, PTPN11 (SHP2), or KRAS Q61X.

In some embodiments, the solid tumor is an advanced or metastatic non-small cell lung cancer (NSCLC) caused by a mutation in KRAS. In some embodiments, the solid tumor is an advanced or metastatic non-small cell lung cancer (NSCLC) caused by a mutation in KRAS, provided that the tumor is other than caused by one or more additional activating mutations in BRAF V600X, PTPN11 (SHP2), or KRAS Q61X. In some embodiments, the solid tumor is a KRAS G12C-positive solid tumor. In some embodiments, the solid tumor is an advanced or metastatic KRASG12C-positive non-small cell lung cancer (NSCLC).

The solid tumor can also be any tumor that is resistant to the treatment of a KRAS G12C inhibitor (e.g., sotorasib (AMG 510), adagrasib (MRTX-849), MRTX1257, ARS-853, ARS-1620, JNJ-74699157 (ARS-3248), JDQ443, GDC-6036, JAB-21822, BI 1823911, MK-1084, LY3537982, and LY3499446). In some embodiments, the solid tumor is resistant to a KRAS G12C inhibitor. In some embodiments, the solid tumor is characterized by intrinsic and/or acquired resistance to a KRAS G12C inhibitor as defined and described herein. In some embodiments, the solid tumor is a KRAS G12C-positive solid tumor resistant to a KRAS G12C inhibitor. In some embodiments, the solid tumor is a KRAS G12C-positive solid tumor characterized by intrinsic and/or acquired resistance to a KRAS G12C inhibitor. In some embodiments, the solid tumor is resistant to the treatment of a KRAS G12C inhibitor selected from the group consisting of sotorasib (AMG 510), adagrasib (MRTX-849), MRTX1257, ARS-853, ARS-1620, JNJ-74699157 (ARS-3248), JDQ443, GDC-6036, JAB-21822, BI 1823911, MK-1084, LY3537982, and LY3499446. In some embodiments, the solid tumor is resistant to sotorasib (AMG 510). In some embodiments, the solid tumor is resistant to adagrasib (MRTX-849). In some embodiments, the solid tumor is a KRAS-positive solid tumor resistant to the treatment of a KRAS G12C inhibitor selected from the group consisting of sotorasib (AMG 510), adagrasib (MRTX-849), MRTX1257, ARS-853, ARS-1620, JNJ-74699157(ARS-3248), JDQ443, GDC-6036, JAB-21822, BI 1823911, MK-1084, LY3537982, and LY3499446. In some embodiments, the solid tumor is a KRAS-positive solid tumor resistant to sotorasib (AMG 510). In some embodiments, the solid tumor is a KRAS-positive solid tumor resistant to adagrasib (MRTX-849). In some embodiments, the solid tumor is a KRAS G12C-positive solid tumor resistant to the treatment of a KRAS G12C inhibitor selected from the group consisting of sotorasib (AMG 510), adagrasib (MRTX-849), MRTX1257, ARS-853, ARS-1620, JNJ-74699157 (ARS-3248), JDQ443, GDC-6036, JAB-21822, BI 1823911, MK-1084, LY3537982, and LY3499446. In some embodiments, the solid tumor is a KRAS G12C-positive solid tumor resistant to sotorasib (AMG 510). In some embodiments, the solid tumor is a KRAS G12C-positive solid tumor resistant to adagrasib (MRTX-849).

In some embodiments, the cancer (e.g., solid tumor) is characterized by one or more mutations in the MAPK pathway, such as one or more mutations in KRAS, NRAS, HRAS, CRAF, BRAF, NRAF, MAPK/ERK, MAPKK/MEK, NF1, EGFR, IGFR, PDGFR, VEGFR, FGFR, CCKR, NGFR, EphR, AXLR, KEAP-1, TIE receptor, RYK receptor, DDR receptor, RET receptor, ROS receptor, LTK receptor, ROR receptor, MuSK receptor, or a combination thereof.

In some embodiments, the cancer or solid tumor is resistant to the treatment of an inhibitor that targets, decreases, or inhibits synthesis, expression, or biological activity in the MAPK pathway, such as an inhibitor targeting one or more of KRAS, NRAS, HRAS, CRAF, BRAF, NRAF, MAPK/ERK, MAPKK/MEK, NF1, EGFR, IGFR, PDGFR, VEGFR, FGFR, CCKR, NGFR, EphR, AXLR, KEAP-1, TIE receptor, RYK receptor, DDR receptor, RET receptor, ROS receptor, LTK receptor, ROR receptor, MuSK receptor, or a combination thereof. In some embodiments, the cancer or solid tumor is characterized by intrinsic and/or acquired resistance to an inhibitor that targets, decreases, or inhibits synthesis, expression, or biological activity in the MAPK pathway, such as an inhibitor targeting one or more of KRAS NRAS, HRAS, CRAF, BRAF, NRAF, MAPK/ERK, MAPKK/MEK, NF1, IGFR, PDGFR, VEGFR, FGFR, CCKR, NGFR, EphR, AXLR, KEAP-1, TIE receptor, RYK receptor, DDR receptor, RET receptor, ROS receptor, LTK receptor, ROR receptor, MuSK receptor, or a combination thereof. Examples of MEK inhibitors include cobimetinib, trametinib, binimetinib, mirdametinib, and selumetinib. Examples of BRAF inhibitors include sorafenib, regorafenib, vemurafenib, encorafenib, and dabrafenib. Examples of EGFR inhibitors include erlotinib, cetuximab, panitumumab, vandetanib, afatinib, gefitinib, osimertinib, necitumumab, brigatinib, neratinib, dacomitinib, amivantamab (JNJ-61186372), mobocertinib (TAK-788), BLU-945, varlitinib, tarloxitinib, poziotinib, and lapatinib.

In any one of embodiments, a standard of care or curative therapy is unavailable for treating the cancer or solid tumor, as described herein.

### III-3: Subject

In some embodiments, the subject is human. In some embodiments, the subject is under the care of a medical practitioner, such as a physician. In some embodiments, the subject has been diagnosed with the cancer. In some embodiments, the subject has relapsed. In some embodiments, the subject has previously entered remission. In some embodiments, the subject has previously undergone, is undergoing, or will undergo a monotherapy course of treatment. In some embodiments, the subject has previously undergone, is undergoing, or will undergo radiation therapy. In some embodiments, the subject has previously undergone, is undergoing, or will undergo immunotherapy. In some embodiments, the subject has previously undergone, is undergoing, or will undergo chemotherapy. In some embodiments, the subject has previously undergone, is undergoing, or will undergo a platinum-based chemotherapy. In some embodiments, the subject has previously undergone, is undergoing, or will undergo a therapeutic regimen comprising administration of a KRAS modulator (e.g., KRAS inhibitor). In some embodiments, the subject has previously undergone, is undergoing, or will undergo a therapeutic regimen comprising administration of an anti-PD-1/PD-L1 inhibitor (e.g., checkpoint inhibitor).

The subject can have an advanced (e.g., primary, metastatic, or recurrent) solid tumor with a KRAS G12C mutation (e.g., as described herein) as assessed by molecular diagnostic using an appropriate clinically validated and/or FDA approved test and with no available standard of care or curative therapies. In some embodiments, the subject has a KRAS G12C mutation (e.g., as described herein), as assessed by molecular diagnostic using an appropriate clinically validated and/or FDA approved test within at least two (2) years prior to the admission to the treatment as described herein.

In some embodiments, the subject has cancer characterized by one or more mutations in KRAS (e.g., as described herein), provided that the cancer is characterized by a KRAS G12C mutation. In some embodiments, the subject has cancer characterized by a KRAS G12C mutation that is not characterized by a KRAS Q61X mutation. In some embodiments, the subject has cancer characterized by a KRAS G12C mutation and an additional mutation at codon 13 (e.g., a G13D, G13A, G13C, G13R, G13S, and G13V mutation). In some embodiments, the subject has cancer characterized by a KRAS G12C mutation and an additional mutation at codon 61.

In some embodiments, the subject has one or more mutations in the MAPK pathway. In some embodiments, the one or more mutations in the MAPK pathway are one or more mutations other than a BRAF mutation comprising V600X mutation, provided that the subject also has a KRAS G12C mutation. In some embodiments, the subject has one or more mutations in the MAPK pathway selected from the group consisting of one or more mutations in NRAS, HRAS, CRAF, BRAF, NRAF, MAPK/ERK, MAPK/MEK, NF1, IGFR, PDGFR, VEGFR, FGFR, CCKR, NGFR, EphR, AXLR, TIE receptor, RYK receptor, DDR receptor, RET receptor, ROS receptor, LTK receptor, ROR receptor, and MuSK receptor, provided that the subject also has a KRAS G12C mutation. In some embodiments, the subject has a mutation in NRAS. In some embodiments, the subject has a mutation in HRAS. In some embodiments, the subject has a mutation in CRAF. In some embodiments, the subject has a mutation in BRAF (except for V600X mutation). In some embodiments, the subject has a mutation in NRAF. In some embodiments, the subject has a mutation in MAPK/ERK. In some embodiments, the subject has a mutation in MAPKK/MEK. In some embodiments, the subject has a mutation in NF1. In some embodiments, the subject has a mutation in IGFR. In some embodiments, the subject has a mutation in PDGFR. In some embodiments, the subject has a mutation in VEGFR. In some embodiments, the subject has a mutation in FGFR. In some embodiments, the subject has a mutation in CCKR. In some embodiments, the subject has a mutation in NGFR. In some embodiments, the subject has a mutation in EphR. In some embodiments, the subject has a mutation in AXLR. In some embodiments, the subject has a mutation in TIE receptor. In some embodiments, the subject has a mutation in RYK receptor. In some embodiments, the subject has a mutation in DDR receptor. In some embodiments, the subject has a mutation in RET receptor. In some embodiments, the subject has a mutation in ROS receptor. In some embodiments, the subject has a mutation in LTK receptor. In some embodiments, the subject has a mutation in ROR receptor. In some embodiments, the subject has a mutation in MuSK receptor.

In some embodiments, the subject has a mutation in EGFR, provided that the subject also has a KRAS G12C mutation. In some embodiments, subject has an EGFR mutation including an EGFR exon 19 deletion, exon 20 insertion, L858X mutation, T790X mutation, C797X mutation, G719X mutation, L861X mutation, S768X mutation, E709X mutation, or any combination thereof. In some embodiments, subject has an EGFR mutation including an EGFR exon 19 deletion, and/or exon 20 insertion. In some embodiments, subject has an EGFR exon 19 deletion. In some embodiments, subject has an EGFR exon 20 insertion.

In some embodiments, the subject does not have a mutation in PTPN11, such as an E76K mutation.

In some embodiments, the subject has the solid tumor progressed or recurred on or after at least one prior line of a systemic therapy including a platinum-based doublet chemotherapy and/or an anti-PD-1/PD-L1 therapy, each of which is given in monotherapy or both of which are given in combination therapy.

In some embodiments, the subject has a measurable disease according to response evaluation criteria in solid tumors (RECIST). In some embodiments, treatment of the subject with the compound of formula (I) or (10b) and sotorasib causes a measurable change in disease state according to RECIST.

In some embodiments, the subject has not previously participated in an interventional clinical study within a period of at least about four (4) weeks or five (5) half-lives of an agent used in the interventional clinical study, whichever is shorter, prior to initiation of the treatment with the compound of formula (I) or (10b) in combination with sotorasib.

In some embodiments, the subject has not previously received a radiotherapy or a proton therapy including i) a limited field of radiation for palliation within a period of about one (1) week, or ii) a radiation to more than about 30% of bone marrow or a wide field of radiation within a period of about four (4) weeks, prior to initiation of the treatment with the compound of formula (I) or (10b) in combination with sotorasib.

In some embodiments, the subject has not taken or is not taking a) one or more of strong or moderate inducers or inhibitors of CYP3A4 and/or P-gp inducers (including herbal supplements or food products containing grapefruit juice, star fruit, or Seville oranges) (e.g., as described in Example 3) within a period of 14 days or five (5) half-lives, whichever is longer prior to initiation of the treatment with the compound of formula (I) or (10b) in combination with sotorasib; b) a drug that is a known substrate of CYP3A4 and/or P-gp within a period of 14 days or five (5) half-lives, whichever is longer prior to initiation of the treatment with the compound of formula (I) or (10b) in combination with sotorasib; and/or one or more acid reducing agents, such as proton pump inhibitors (PPIs) or H2 receptor antagonists within a period of 3 days of initiation of the treatment with the compound of formula (I) or (10b) in combination with sotorasib.

In some embodiments, the subject does not have inadequate organ functions including adequate hematological, renal, hepatic, and coagulating functions, as defined below:

### Hematological

a. Absolute neutrophil count <1,500/μL;
b. Platelets <100,000/μL; and
c. Hemoglobin <9 g/dL without transfusion for ≤2 weeks or erythropoiesis-stimulating agents (e.g., Epo, Procrit) for ≤6 weeks.

### Renal

d. Serum creatinine >1.5×ULN, unless creatinine clearance ≥40 mL/min (measured or calculated using the Cockcroft-Gault formula)

### Hepatic

e. Serum total bilirubin ≥2× institutional upper limit of normal (ULN) or ≥3.0× institutional ULN if the patient has a diagnosis of Gilbert syndrome or hemolytic anemia as confirmed by the investigator; and
f. Aspartate aminotransferase/serum glutamic-oxaloacetic transaminase (AST/SGOT) and/or alanine aminotransferase/serum glutamic-pyruvic transaminase (ALT/SGPT) >2.5×ULN.

### Coagulation

g. International normalized ratio (INR) or prothrombin time (PT) >1.5×ULN unless the patient is receiving anticoagulant therapy and as long as PT or activated partial thromboplastin time (aPTT) is within the therapeutic range of intended use of anticoagulants; and
h. Activated partial thromboplastin time >1.5×ULN unless the patient is receiving anticoagulant therapy and as long as PT or aPTT is within the therapeutic range of intended use of anticoagulants.

In some embodiments, the subject does not have active hepatitis B infection, hepatitis C infection, or human immunodeficiency virus (HIV) infection with measurable viral load.

In some embodiments, the subject does not have has a life-threatening illness, medical condition, an active uncontrolled infection, or an organ system dysfunction (e.g., ascites, coagulopathy, or encephalopathy).

In some embodiments, the subject does not have one or more cardiac-related diseases or findings:
a) History of significant cardiovascular disease (e.g., cerebrovascular accident, myocardial infarction or unstable angina), within the last 6 months before starting the treatment;
b) Clinically significant cardiac disease, including New York Heart Association Class II or higher heart failure;
c) History of left ventricular ejection fraction (LVEF) <50% within the previous 12 months before starting the treatment;
d) Resting corrected QT interval (QTc) >470 msec, derived as the averaged from three electrocardiograms (ECGs), using the ECG machines provided; and/or
e) Any clinically significant abnormalities in rhythm, conduction, or morphology of resting ECG (e.g., third degree heart block, Mobitz Type II heart block, ventricular arrhythmias, uncontrolled atrial fibrillation).

In some embodiments, the subject has not been diagnosed of an additional invasive malignancy within the previous 3 years, provided that the additional invasive malignancy is other than curatively treated non-melanomatous skin cancer, superficial urothelial carcinoma, in situ cervical cancer, or any other curatively treated malignancy that is not expected to require treatment for recurrence during the course of the treatment with the compound of formula (I) or (10b) in combination with sotorasib.

In some embodiments, the subject does not have one or more untreated brain metastases from non-brain tumors.

In some embodiments, the subject who has had brain metastases resected or have received radiation therapy ending at least 4 weeks prior to the initiation of the treatment (e.g., Cycle 1, Day 1) with the compound of formula (I) or (10b) in combination with sotorasib is eligible, provided that the subject meets all of the following criteria prior to the initiation of the treatment: a) residual neurological symptoms related to the CNS treatment Grade ≤2; b) on a stable or decreasing dose of ≤10 mg daily prednisone (or equivalent) for at least 2 weeks prior to Cycle 1, Day 1, if applicable; and c) follow-up magnetic resonance imaging (MRI) within 4 weeks prior to Cycle 1, Day 1 shows no new lesions appearing.

In some embodiments, the subject has not undergone a major surgery within 4 weeks prior to the enrollment for the treatment with the compound of formula (I) or (10b) in combination with sotorasib, provided that the surgery or procedure is other than peripherally inserted central catheter line placement, thoracentesis, paracentesis, biopsies, or abscess drainage.

In some embodiments, the subject does not have a history of hypersensitivity to sotorasib or the compound of formula (I) or (10b), active or inactive excipients of sotorasib or the compound of formula (I) or (10b) or drugs with a similar chemical structure or class to either sotorasib or the compound of formula (I) or (10b), dependent on which combination the subject could receive.

In some embodiments, the subject does not have one or more additional activating mutations in BRAF V600X, PTPN11 (SHP2), and/or KRAS Q61X. In some embodiments, the subject does not have a tumor harboring one or more additional activating mutations in BRAF V600X, PTPN11 (SHP2), and/or KRAS Q61X.

In some embodiments, the subject is not previously treated with a KRAS G12C inhibitor (e.g., as described herein). In some embodiments, the subject is not previously treated with sotorasib. In some embodiments, the subject was previously treated with sotorasib. In some embodiments, the subject was previously treated with a KRAS G12C inhibitor other than sotorasib. In some embodiments, the subject is not previously treated with a PTPN11 inhibitor (e.g., SHP2 inhibitor), provided that the PTPN11 inhibitor is other than the compound of formula (I) or (10b). In some embodiments, the subject is not previously treated with a PTPN11 inhibitor selected from the group consisting of TNO-155, RMC-4630, RLY-1971, JAB-3068, JAB-3312, PF-07284892, and ERAS601. In some embodiments, the subject is not previously treated with the compound of formula (I) or (10b). In some embodiments, the subject has previously been treated with a SHP2 inhibitor including any one of TNO-155, RMC-4630, RLY-1971, JAB-3068, JAB-3312, PF-07284892, ERAS601, and the compound of formula (I) or (10b). In some embodiments, the subject was previously treated with the compound of formula (I) or (10b). In some embodiments, the subject was previously treated with a PTPN11 inhibitor other than the compound of formula (I) or (10b).

In some embodiments, the subject does not have a gastrointestinal illness (e.g., post gastrectomy, short bowel syndrome, uncontrolled Crohn's disease, celiac disease with villous atrophy, or chronic gastritis), which may preclude absorption of the compound of formula (I) or (10b).

In some embodiments, the subject is not on dialysis.

In some embodiments, the subject does not have a history of allogenic bone marrow transplant.

Further inclusion and exclusion criteria for subjects who may benefit from treatment with the compound of formula (I) or (10b) in combination with sotorasib, such as subjects enrolled in a Phase 1 Study of the SHP2 Inhibitor Compound (10b) in Combination with sotorasib, are described in Example 3.

In some embodiments, the subject meets all of inclusion criteria of 1) to 11) as described in Example 3. In some embodiments, the subject meets all of inclusion criteria of 1) to 11) as described in Example 3, provided that the subject does not meet any one of exclusion criteria of 1) to 17) as described in Example 3.

### III-4: Treatment Cycle and Dose Adjustment

Treatment with the compound (I) or (10b) in combination with sotorasib can include one or more treatment cycles (e.g., at least 1, 2, 3, or more treatment cycles). In some embodiments, the treatment includes one or more treatment cycles (e.g., at least 1, 2, 3, or more treatment cycles). In some embodiments, the treatment includes at least 2, 3, or more treatment cycles. In some embodiments, the treatment includes 2 to 3 treatment cycles. In some embodiments, the treatment includes 3 treatment cycles. In some embodiments, the treatment includes more than 3 treatment cycles.

In some embodiments, each of one or more treatment cycles has a duration of about 28 days; and the compound of formula (I) or (10b) is administered daily. In some embodiments, each of one or more treatment cycles has a duration of about 28 days; and sotorasib is administered daily. In some embodiments, each of one or more treatment cycles has a duration of about 28 days; the compound of formula (I) or (10b) is administered daily; and sotorasib is administered daily.

The treatment may include a dose escalation period, during which, after a previous treatment cycle, a dose of the compound of formula (I) or (10b) or sotorasib can be adjusted (e.g., dose escalation or de-escalation) or retained. Dose adjustment may be based at least in part on a safety evaluation (e.g., a dose-limiting toxicity (DLT) assessment).

In some embodiments, the subject begins treatment with the compound of formula (I) or (10b) and sotorasib at a first compound dose level and a first sotoroasib dose level, and is subsequently treated at a second compound dose level and a second sotorasib dose level, where the second compound dose level differs from the first compound dose level and/or the second sotorasib dose level differs from the first sotorasib dose level. In some embodiments, the second sotorasib dose level is lower than the first sotorasib dose level. In some embodiments, the second sotorasib dose level is higher than the first sotorasib dose level. In some embodiments, the second compound dose level is lower than the first compound dose level. In some embodiments, the second compound dose level is higher than the first compound dose level.

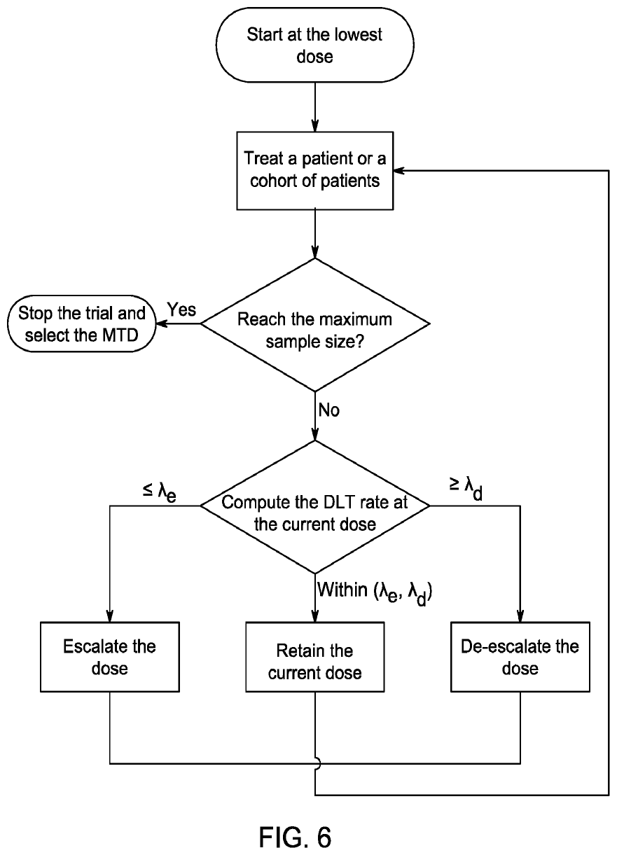

In some embodiments, the administration of sotorasib in combination with the compound of formula (I) or (10b) includes one or more dose escalations (e.g., dose increases), dose retentions, or dose de-escalations (e.g., dose reductions) of sotorasib and/or the compound of formula (I) or (10b). In some embodiments, the administration of sotorasib in combination with the compound of formula (I) or (10b) includes one or more dose escalations, dose retentions, or dose de-escalations of sotorasib. In some embodiments, the administration of sotorasib in combination with the compound of formula (I) or (10b) includes one or more dose de-escalations of sotorasib. In some embodiments, the administration of sotorasib in combination with the compound of formula (I) or (10b) includes one or more dose escalations of sotorasib. In some embodiments, the administration of sotorasib in combination with the compound of formula (I) or (10b) includes one or more dose escalations, dose retentions, or dose de-escalations of the compound of formula (I) or (10b). In some embodiments, the administration of sotorasib in combination with the compound of formula (I) or (10b) includes one or more dose de-escalations (e.g., dose reductions) of the compound of formula (I) or (10b). In some embodiments, the administration of sotorasib in combination with the compound of formula (I) or (10b) includes one or more dose escalations (e.g., dose increases) of the compound of formula (I) or (10b). In some embodiments, the administration of sotorasib in combination with the compound of formula (I) or (10b) includes one or more dose escalations (e.g., dose increases), dose retentions, or dose de-escalations (e.g., dose reductions) of sotorasib and/or the compound of formula (I) or (10b), each of which is determined by a safety or dose-limiting toxicity (DLT) assessment (e.g., relevant to a cohort of subjects). In some embodiments, the administration of sotorasib in combination with the compound of formula (I) or (10b) includes one or more dose escalations, dose retentions, or dose de-escalations of the compound of formula (I) or (10b), each of which is determined by a dose-limiting toxicity (DLT) assessment, as described in Example 3 and FIG. 6.

In some embodiments, the administration of the compound of formula (I) or (10b) includes a dose escalation after a previous treatment cycle, when a dose-limiting toxicity (DLT) rate is less than, e.g., about 19.7% as determined by a DLT assessment (e.g., relevant to a cohort of subjects). In some embodiments, the administration of the compound of formula (I) or (10b) includes a dose escalation in a second treatment cycle after a first treatment cycle, when a dose-limiting toxicity (DLT) rate is less than, e.g., about 19.7% as determined by a DLT assessment (e.g., relevant to a cohort of subjects). In some embodiments, the administration of the compound of formula (I) or (10b) includes a dose escalation in a third treatment cycle after a second treatment cycle, when a dose-limiting toxicity (DLT) rate is less than, e.g., as about 19.7% determined by a DLT assessment (e.g., relevant to a cohort of subjects).

In some embodiments, the administration of the compound of formula (I) or (10b) includes a dose de-escalation after a previous treatment cycle, when a dose-limiting toxicity rate is more than, e.g., about 29.8% as determined by a DLT assessment (e.g., relevant to a cohort of subjects). In some embodiments, the administration of the compound of formula (I) or (10b) includes a dose de-escalation in a second treatment cycle after a first treatment cycle, when a dose-limiting toxicity rate is more than, e.g., about 29.8% as determined by a DLT assessment (e.g., relevant to a cohort of subjects). In some embodiments, the administration of the compound of formula (I) or (10b) includes a dose de-escalation in a third treatment cycle after a second treatment cycle, when a dose-limiting toxicity rate is more than, e.g., about 29.8% as determined by a DLT assessment (e.g., relevant to a cohort of subjects).

In some embodiments, the administration of the compound of formula (I) or (10b) includes a dose retention after a previous treatment cycle, when a dose-limiting toxicity rate is in a range of from about 21.9% to about 29.8% determined by a DLT assessment (e.g., relevant to a cohort of subjects). In some embodiments, the administration of the compound of formula (I) or (10b) includes a dose retention in a second treatment cycle after a first treatment cycle, when a dose-limiting toxicity rate is in a range of from about 21.9% to about 29.8% determined by a DLT assessment (e.g., relevant to a cohort of subjects). In some embodiments, the administration of the compound of formula (I) or (10b) includes a dose retention in a third treatment cycle after a second treatment cycle, when a dose-limiting toxicity rate is in a range of from about 21.9% to about 29.8% determined by a DLT assessment (e.g., relevant to a cohort of subjects).

After the dose escalation period, the treatment further includes a dose expansion/optimization period. In some embodiments of the dose expansion/optimization period, the compound of formula (I) or (10b) is administered at a dose regimen (e.g., Dose Regimen 1 or Dose Regimen 2) determined during the dose escalation period.

In some embodiments, the administration of the compound of formula (I) or (10b) includes one or more dose adjustments. In some embodiments, the administration of the compound of formula (I) or (10b) includes one or more dose adjustments during the dose expansion/optimization period. In some embodiments, the administration of the compound of formula (I) or (10b) optionally includes one or more dose adjustments during the dose expansion/optimization period; and the one or more dose adjustments are determined according to a safety evaluation by Safety Review Committee (SRC).

In some embodiments, dosing adjustments, delays, and discontinuations of the compound of formula (I) or (10b) and/or sotorasib are further based on the criteria of Example 3.

### III-5: Therapeutically Effective Amount/Administration

The compound of formula (I) or (10b) and sotorasib can be provided in jointly therapeutically effective amounts or in synergistically effective amounts, or each of which can be used at a dose different than when each is used alone. In some embodiments, the compound of formula (I) or (10b) and sotorasib are provided in jointly therapeutically effective amounts. In some embodiments, the compound of formula (I) or (10b) and sotorasib are provided in synergistically effective amounts.

In some embodiments, the compound of formula (I) or (10b) and/or sotorasib is used at a dose different than when it is used alone (e.g., as in a monotherapy treatment). In some embodiments, the compound of formula (I) or (10b) and sotorasib are each used at a dose different than when each is used alone. In some embodiments, the compound of formula (I) or (10b) and sotorasib are each used at a dose lower than when each is used alone. In some embodiments, the compound of formula (I) or (10b) is used at a dose lower than when it is used alone. In some embodiments, sotorasib is used at a dose lower than when it is used alone. In some embodiments, the compound of formula (I) or (10b) is used at a dose higher than when it is used alone. In some embodiments, sotorasib is used at a dose higher than when it is used alone.

The compound of formula (I) or (10b) and sotorasib can be administered concomitantly or sequentially. In some embodiments, the compound of formula (I) or (10b) and sotorasib are administered concomitantly. In some embodiments, the compound of formula (I) or (10b) and sotorasib are administered in a pharmaceutical composition including the compound of formula (I) or (10b) and sotorasib. In some embodiments, the compound of formula (I) or (10b) and sotorasib are administered sequentially. In some embodiments, the compound of formula (I) or (10b) is administered prior to the administration of sotorasib. In some embodiments, the compound of formula (I) or (10b) is administered after the administration of sotorasib.

In some embodiments, the therapeutically effective amount of the compound of formula (I) or (10b) is a total daily dosage of no more than about 2000 mg, on a salt-free and anhydrous basis. In some embodiments, the therapeutically effective amount is a total daily dosage of from about 100 mg to about 2000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 100 mg to about 700 mg, from about 150 mg to about 700 mg, from about 200 mg to about 700 mg, from about 250 mg to about 700 mg, from about 300 mg to about 700 mg, from about 3 mg to about 700 mg, from about 400 mg to about 700 mg, from about 450 mg to about 700 mg, from about 500 mg to about 700 mg, from about 550 mg to about 700 mg, from about 100 mg to about 550 mg, from about 150 mg to about 550 mg, from about 200 mg to about 550 mg, from about 250 mg to about 550 mg, from about 300 mg to about 550 mg, from about 350 mg to about 550 mg, from about 400 mg to about 550 mg, from about 450 mg to about 550 mg, from about 100 mg to about 400 mg, from about 150 mg to about 400 mg, from about 200 mg to about 400 mg, from about 250 mg to about 400 mg, or from about 300 mg to about 400 mg of the compound of formula (I) or (10b), on a salt-free and anhydrous basis, or any useful range therein.

In some embodiments, the therapeutically effective amount is a total daily dosage of from about 250 mg to about 400 mg, from about 400 mg to about 550 mg, or from about 550 mg to about 700 mg of the compound of formula (I) or (10b), on a salt-free and anhydrous basis, or any useful range therein.

In some embodiments, the therapeutically effective amount of the compound of formula (10b) is a total daily dosage of about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg of the compound of formula (10b), on a salt-free and anhydrous basis. In some embodiments, the therapeutically effective amount of the compound of formula (10b) is a total daily dosage of about 250 mg, about 400 mg, or about 550 mg of the compound of formula (10b), on a salt-free and anhydrous basis. In some embodiments, the therapeutically effective amount is a total daily dosage of about 250 mg of the compound of formula (10b), on a salt-free and anhydrous basis. In some embodiments, the therapeutically effective amount is a total daily dosage of about 400 mg of the compound of formula (10b), on a salt-free and anhydrous basis. In some embodiments, the therapeutically effective amount is a total daily dosage of about 550 mg of the compound of formula (10b), on a salt-free and anhydrous basis.

In some embodiments, the therapeutically effective amount of sotorasib is a total daily dosage of from about 120 mg to about 960 mg, from about 240 mg to about 960 mg, from about 360 mg to about 960 mg, from about 480 mg to about 960 mg, from about 600 mg to about 960 mg, from about 720 mg to about 960 mg, from about 840 mg to about 960 mg, from about 120 mg to about 840 mg, from about 240 mg to about 840 mg, from about 360 mg to about 840 mg, from about 480 mg to about 840 mg, from about 600 mg to about 840 mg, from about 720 mg to about 840 mg, from about 120 mg to about 720 mg, from about 240 mg to about 720 mg, from about 360 mg to about 720 mg, from about 480 mg to about 720 mg, from about 600 mg to about 720 mg, from about 120 mg to about 600 mg, from about 240 mg to about 600 mg, from about 360 mg to about 600 mg, from about 480 mg to about 600 mg, from about 120 mg to about 480 mg, from about 240 mg to about 480 mg, from about 360 mg to about 480 mg, from about 120 mg to about 360 mg, from about 240 mg to about 360 mg, or from about 120 mg to about 240 mg of sotorasib.

In some embodiments, the therapeutically effective amount of sotorasib is a total daily dosage of about 120 mg, about 240 mg, about 360 mg, about 480 mg, about 600 mg, about 720 mg, about 840 mg, or about 960 mg of sotorasib.

In some embodiments, the therapeutically effective amount of the compound of formula (10b) is a total daily dosage of no more than about 2000 mg, on a salt-free and anhydrous basis; and the therapeutically effective amount of sotorasib is a total daily dosage of no more than about 960 mg. In some embodiments, the therapeutically effective amount of the compound of formula (10b) is a total daily dosage of no more than about 2000 mg, on a salt-free and anhydrous basis; and the therapeutically effective amount of sotorasib is a total daily dosage of about 960 mg. In some embodiments, the therapeutically effective amount of the compound of formula (10b) is a total daily dosage of about 100 mg to about 2000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 100 mg to about 700 mg, from about 150 mg to about 700 mg, from about 200 mg to about 700 mg, from about 250 mg to about 700 mg, from about 300 mg to about 700 mg, from about 350 mg to about 700 mg, from about 400 mg to about 700 mg, from about 450 mg to about 700 mg, from about 500 mg to about 700 mg, from about 550 mg to about 700 mg, from about 100 mg to about 550 mg, from about 150 mg to about 550 mg, from about 200 mg to about 550 mg, from about 250 mg to about 550 mg, from about 300 mg to about 550 mg, from about 350 mg to about 550 mg, from about 400 mg to about 550 mg, from about 450 mg to about 550 mg, from about 100 mg to about 400 mg, from about 150 mg to about 400 mg, from about 200 mg to about 400 mg, from about 250 mg to about 400 mg, or from about 300 mg to about 400 mg of the compound of formula (10b), on a salt-free and anhydrous basis, or any useful range therein; and the therapeutically effective amount of sotorasib is a total daily dosage of from about 120 mg to about 960 mg, from about 240 mg to about 960 mg, from about 360 mg to about 960 mg, from about 480 mg to about 960 mg, from about 600 mg to about 960 mg, from about 720 mg to about 960 mg, from about 840 mg to about 960 mg, from about 120 mg to about 840 mg, from about 240 mg to about 840 mg, from about 360 mg to about 840 mg, from about 480 mg to about 840 mg, from about 600 mg to about 840 mg, from about 720 mg to about 840 mg, from about 120 mg to about 720 mg, from about 240 mg to about 720 mg, from about 360 mg to about 720 mg, from about 480 mg to about 720 mg, from about 600 mg to about 720 mg, from about 120 mg to about 600 mg, from about 240 mg to about 600 mg, from about 360 mg to about 600 mg, from about 480 mg to about 600 mg, from about 120 mg to about 480 mg, from about 240 mg to about 480 mg, from about 360 mg to about 480 mg, from about 120 mg to about 360 mg, from about 240 mg to about 360 mg, or from about 120 mg to about 240 mg of sotorasib. In some embodiments, the therapeutically effective amount is a total daily dosage of about 100 mg to about 2000 mg, from about 150 mg to about 1000 mg, from about 250 mg to about 1000 mg, from about 400 mg to about 1000 mg, from about 250 mg to about 700 mg, from about 250 mg to about 550 mg, from about 250 mg to about 400 mg, from about 400 mg to about 550 mg, or from about 550 mg to about 700 mg of the compound of formula (10b), on a salt-free and anhydrous basis, or any useful range therein; and the therapeutically effective amount of sotorasib is a total daily dosage of about 120 mg, about 240 mg, about 360 mg, about 480 mg, about 600 mg, about 720 mg, about 840 mg, or about 960 mg. In some embodiments, the therapeutically effective amount is a total daily dosage of from about 250 mg to about 400 mg, from about 400 mg to about 550 mg, or from about 550 mg to about 700 mg of the compound of formula (10b), on a salt-free and anhydrous basis, or any useful range therein; and the therapeutically effective amount of sotorasib is a total daily dosage of about 120 mg, about 240 mg, about 360 mg, about 480 mg, about 600 mg, about 720 mg, about 840 mg, or about 960 mg.

In some embodiments, the therapeutically effective amount of the compound of formula (10b) is a total daily dosage of about 250 mg, about 400 mg, or about 550 mg of the compound of formula (10b), on a salt-free and anhydrous basis; and the therapeutically effective amount of sotorasib is a total daily dosage of about 120 mg, about 240 mg, about 360 mg, about 480 mg, about 600 mg, about 720 mg, about 840 mg, or about 960 mg. In some embodiments, the therapeutically effective amount is a total daily dosage of about 250 mg of the compound of formula (10b), on a salt-free and anhydrous basis; and the therapeutically effective amount of sotorasib is a total daily dosage of about 960 mg. In some embodiments, the therapeutically effective amount is a total daily dosage of about 400 mg of the compound of formula (10b), on a salt-free and anhydrous basis; and the therapeutically effective amount of sotorasib is a total daily dosage of about 960 mg. In some embodiments, the therapeutically effective amount is a total daily dosage of about 550 mg of the compound of formula (10b), on a salt-free and anhydrous basis; and the therapeutically effective amount of sotorasib is a total daily dosage of about 960 mg. In some embodiments, the therapeutically effective amount is a total daily dosage of about 250 mg of the compound of formula (10b), on a salt-free and anhydrous basis; and the therapeutically effective amount of sotorasib is a total daily dosage of about 840 mg. In some embodiments, the therapeutically effective amount is a total daily dosage of about 400 mg of the compound of formula (10b), on a salt-free and anhydrous basis; and the therapeutically effective amount of sotorasib is a total daily dosage of about 840 mg. In some embodiments, the therapeutically effective amount is a total daily dosage of about 550 mg of the compound of formula (10b), on a salt-free and anhydrous basis; and the therapeutically effective amount of sotorasib is a total daily dosage of about 840 mg. In some embodiments, the therapeutically effective amount is a total daily dosage of about 250 mg of the compound of formula (10b), on a salt-free and anhydrous basis; and the therapeutically effective amount of sotorasib is a total daily dosage of about 720 mg. In some embodiments, the therapeutically effective amount is a total daily dosage of about 400 mg of the compound of formula (10b), on a salt-free and anhydrous basis; and the therapeutically effective amount of sotorasib is a total daily dosage of about 720 mg. In some embodiments, the therapeutically effective amount is a total daily dosage of about 550 mg of the compound of formula (10b), on a salt-free and anhydrous basis; and the therapeutically effective amount of sotorasib is a total daily dosage of about 720 mg. In some embodiments, the therapeutically effective amount is a total daily dosage of about 250 mg of the compound of formula (10b), on a salt-free and anhydrous basis; and the therapeutically effective amount of sotorasib is a total daily dosage of about 600 mg. In some embodiments, the therapeutically effective amount is a total daily dosage of about 400 mg of the compound of formula (10b), on a salt-free and anhydrous basis; and the therapeutically effective amount of sotorasib is a total daily dosage of about 600 mg. In some embodiments, the therapeutically effective amount is a total daily dosage of about 550 mg of the compound of formula (10b), on a salt-free and anhydrous basis; and the therapeutically effective amount of sotorasib is a total daily dosage of about 600 mg. In some embodiments, the therapeutically effective amount is a total daily dosage of about 250 mg of the compound of formula (10b), on a salt-free and anhydrous basis; and the therapeutically effective amount of sotorasib is a total daily dosage of about 480 mg. In some embodiments, the therapeutically effective amount is a total daily dosage of about 400 mg of the compound of formula (10b), on a salt-free and anhydrous basis; and the therapeutically effective amount of sotorasib is a total daily dosage of about 480 mg. In some embodiments, the therapeutically effective amount is a total daily dosage of about 550 mg of the compound of formula (10b), on a salt-free and anhydrous basis; and the therapeutically effective amount of sotorasib is a total daily dosage of about 480 mg. In some embodiments, the therapeutically effective amount is a total daily dosage of about 250 mg of the compound of formula (10b), on a salt-free and anhydrous basis; and the therapeutically effective amount of sotorasib is a total daily dosage of about 360 mg. In some embodiments, the therapeutically effective amount is a total daily dosage of about 400 mg of the compound of formula (10b), on a salt-free and anhydrous basis; and the therapeutically effective amount of sotorasib is a total daily dosage of about 360 mg. In some embodiments, the therapeutically effective amount is a total daily dosage of about 550 mg of the compound of formula (10b), on a salt-free and anhydrous basis; and the therapeutically effective amount of sotorasib is a total daily dosage of about 360 mg. In some embodiments, the therapeutically effective amount is a total daily dosage of about 250 mg of the compound of formula (10b), on a salt-free and anhydrous basis; and the therapeutically effective amount of sotorasib is a total daily dosage of about 240 mg. In some embodiments, the therapeutically effective amount is a total daily dosage of about 400 mg of the compound of formula (10b), on a salt-free and anhydrous basis; and the therapeutically effective amount of sotorasib is a total daily dosage of about 240 mg. In some embodiments, the therapeutically effective amount is a total daily dosage of about 550 mg of the compound of formula (10b), on a salt-free and anhydrous basis; and the therapeutically effective amount of sotorasib is a total daily dosage of about 240 mg. In some embodiments, the therapeutically effective amount is a total daily dosage of about 250 mg of the compound of formula (10b), on a salt-free and anhydrous basis; and the therapeutically effective amount of sotorasib is a total daily dosage of about 120 mg. In some embodiments, the therapeutically effective amount is a total daily dosage of about 400 mg of the compound of formula (10b), on a salt-free and anhydrous basis; and the therapeutically effective amount of sotorasib is a total daily dosage of about 120 mg. In some embodiments, the therapeutically effective amount is a total daily dosage of about 550 mg of the compound of formula (10b), on a salt-free and anhydrous basis; and the therapeutically effective amount of sotorasib is a total daily dosage of about 120 mg.

In general, the compound of formula (I) or (10b) can be administered orally. In some embodiments, the compound of formula (10b) is administered orally. In some embodiments, the compound of formula (10b) in a tablet formulation is administered orally.

In general, sotorasib can be administered orally. In some embodiments, sotorasib is administered orally. In some embodiments, sotorasib in a tablet formulation is administered orally.

In general, the compound of formula (I) or (10b) can be administered once or multiple times (e.g., 2, 3, 4, or more times) daily. In some embodiments, the compound of formula (10b) is administered once, twice, three times, or four times daily. In some embodiments, the compound of formula (10b) is administered once daily. In some embodiments, the compound of formula (10b) is administered twice daily. In some embodiments, the compound of formula (10b) is administered every other day. In some embodiments, the compound of formula (10b) is administered with four days on and three days off (e.g., compound is administered for four consecutive days and then not administered for three consecutive days), five days on and two days off, two days on and five days off, one week on and one week off, two weeks on and one week off, three weeks on and one week off, or a similar schedule.

In general, sotorasib can be administered once, twice, or multiple times (e.g., 2, 3, 4, or more times) daily. In some embodiments, sotorasib is administered once daily.

In some embodiments, the compound of formula (I) or (10b) and sotorasib are each administered orally. In some embodiments, the compound of formula (10b) and sotorasib are each administered orally. In some embodiments, the compound of formula (I) or (10b) is administered once daily; and sotorasib is administered once daily. In some embodiments, the compound of formula (10b) is administered once daily; and sotorasib is administered once daily.

The compound of formula (I) or (10b) can be in an oral dosage form in one or more dosage strengths, where the compound of formula (I) or (10b) is present in an amount of at least about 5 mg, 10 mg, 20 mg, 30 mg, 50 mg, 100 mg, 120 mg, 180 mg, 200 mg, 300 mg, 400 mg, or 500 mg, on a salt-free and anhydrous basis. In some embodiments, the oral dosage form is a tablet formulation in one or more dosage strengths. In some embodiments of the tablet formulation, the compound of formula (10b) is present in an amount of from 30 to 1000 mg, from 30 to 750 mg, from 30 to 500 mg, from 30 to 200 mg, from 30 to 180 mg, from 30 to 120 mg, from 30 to 90 mg, from 50 to 1000 mg, from 50 to 750 mg, from 50 to 500 mg, from 50 to 250 mg, from 100 to 1000 mg, from 100 to 750 mg, from 100 to 500 mg, from 100 to 250 mg, from 200 to 1000 mg, from 200 to 750 mg, from 200 to 500 mg, from 300 to 1000 mg, from 300 to 750 mg, from 300 to 500 mg, from 400 to 1000 mg, from 400 to 750 mg, from 500 to 1000 mg, from 500 to 750 mg, from 600 to 1000 mg, from 5 to 250 mg, or from 5 to 100 mg in each tablet, on a salt-free and anhydrous basis.

In some embodiments of the tablet formulation, the compound of formula (10b) is present in an amount of about 5 mg, 10 mg, 30 mg, 50 mg, 100 mg, 150 mg, 200 mg, 250 mg, 300 mg, 400 mg, 500 mg, 600 mg, 700 mg, 800 mg, 900 mg, or 1000 mg in each tablet, on a salt-free and anhydrous basis. In some embodiments of the tablet formulation, the compound of formula (10b) is present in an amount of about 30 mg, 50 mg, or 100 mg in each tablet, on a salt-free and anhydrous basis. In some embodiments of the tablet formulation, the compound of formula (10b) is present in an amount of about 30 mg in each tablet, on a salt-free and anhydrous basis. In some embodiments of the tablet formulation, the compound of formula (10b) is present in an amount of about 50 mg in each tablet, on a salt-free and anhydrous basis. In some embodiments of the tablet formulation, the compound of formula (10b) is present in an amount of about 100 mg in each tablet, on a salt-free and anhydrous basis.

In some embodiments, the compound of formula (10b) is administered once daily to provide a total daily dosage of no more than about 2000 mg of the compound of formula (10b). In some embodiments, the compound of formula (10b) is administered once daily to provide a total daily dosage of from about 100 mg to about 2000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 100 mg to about 700 mg, from about 150 mg to about 700 mg, from about 200 mg to about 700 mg, from about 250 mg to about 700 mg, from about 300 mg to about 700 mg, from about 350 mg to about 700 mg, from about 400 mg to about 700 mg, from about 450 mg to about 700 mg, from about 500 mg to about 700 mg, from about 550 mg to about 700 mg, from about 100 mg to about 550 mg, from about 150 mg to about 550 mg, from about 200 mg to about 550 mg, from about 250 mg to about 550 mg, from about 300 mg to about 550 mg, from about 350 mg to about 550 mg, from about 400 mg to about 550 mg, from about 450 mg to about 550 mg, from about 100 mg to about 400 mg, from about 150 mg to about 400 mg, from about 200 mg to about 400 mg, from about 250 mg to about 400 mg, or from about 300 mg to about 400 mg of the compound of formula (10b), on a salt-free and anhydrous basis. In some embodiments, the compound of formula (10b) is administered once daily to provide a total daily dosage of from about 100 mg to about 2000 mg, from about 150 mg to about 1000 mg, from about 250 mg to about 1000 mg, from about 400 mg to about 1000 mg, from about 250 mg to about 700 mg, from about 250 mg to about 550 mg, from about 250 mg to about 400 mg, from about 400 mg to about 550 mg, or from about 550 mg to about 700 mg of the compound of formula (10b), on a salt-free and anhydrous basis. In some embodiments, the compound of formula (10b) is administered once daily to provide a total daily dosage of from about 250 mg to about 400 mg, from about 400 mg to about 550 mg, or from about 550 mg to about 700 mg of the compound of formula (10b), on a salt-free and anhydrous basis. In some embodiments, the compound of formula (10b) is administered once daily to provide a total daily dosage of about 250 mg, about 400 mg, about 550 mg of the compound of formula (10b), on a salt-free and anhydrous basis.

Sotorasib can be in an oral dosage form in one or more dosage strengths. In some embodiments, the oral dosage form of sotorasib is a tablet formulation in one or more dosage strengths. In some embodiments, sotorasib is provided as a tablet formulation comprising about 120 mg of sotorasib in each tablet. In some embodiments, sotorasib is provided as a tablet formulation comprising about 60 mg, 120 mg, 240 mg, 360 mg, or more sotorasib in each tablet.

In some embodiments, sotorasib is administered once daily to provide a total daily dosage of no more than about 2000 mg of sotorasib. In some embodiments, sotorasib is administered once daily to provide a total daily dosage of no more than about 960 mg. In some embodiments, sotorasib is administered once daily to provide a total daily dosage of from about 120 mg to about 960 mg, such as about 120 mg, about 240 mg, about 360 mg, about 480 mg, about 600 mg, about 720 mg, about 840 mg, or about 960 mg.

In some embodiments, the compound of formula (10b) is administered once daily to provide a total daily dosage of no more than about 2000 mg of the compound of formula (10b); and sotorasib is administered once daily to provide a total daily dosage of no more than about 960 mg. In some embodiments, the compound of formula (10b) is administered once daily to provide a total daily dosage of from about 100 mg to about 2000 mg, from about 150 mg to about 1000 mg, from about 250 mg to about 1000 mg, from about 400 mg to about 1000 mg, from about 250 mg to about 700 mg, from about 250 mg to about 550 mg, from about 250 mg to about 400 mg, from about 400 mg to about 550 mg, or from about 550 mg to about 700 mg of the compound of formula (10b), on a salt-free and anhydrous basis; and sotorasib is administered once daily to provide a total daily dosage of about 120 mg to about 960 mg, such as about 120 mg, about 240 mg, about 360 mg, about 480 mg, about 600 mg, about 720 mg, about 840 mg, or about 960 mg. In some embodiments, the compound of formula (10b) is administered once daily to provide a total daily dosage of from about 250 mg to about 400 mg, from about 400 mg to about 550 mg, or from about 550 mg to about 700 mg of the compound of formula (10b), on a salt-free and anhydrous basis; and sotorasib is administered once daily to provide a total daily dosage of about 120 mg to about 960 mg, such as about 120 mg, about 240 mg, about 360 mg, about 480 mg, about 600 mg, about 720 mg, about 840 mg, or about 960 mg. In some embodiments, the compound of formula (10b) is administered once daily to provide a total daily dosage of about 250 mg, about 400 mg, about 550 mg of the compound of formula (10b), on a salt-free and anhydrous basis; and sotorasib is administered once daily to provide a total daily dosage of about 960 mg.

In some embodiments, the compound of formula (10b) is administered once daily during each of one or more treatment cycles, as described herein. In some embodiments, sotorasib is administered once daily during each of one or more treatment cycles, as described herein. In some embodiments, the compound of formula (10b) and sotorasib are each administered once daily during each of one or more treatment cycles, as described herein.

In general, the compound of formula (10b) is recommended to be administered to a subject without food (e.g., after an overnight fast (minimum 8 hours) followed by 2 hours of fasting after the dose is taken). The subject is allowed to have water except for one (1) hour before and after the administration and the subject is given with water (e.g., 240 mL) at the administration. In some embodiments, the compound of formula (10b) is administered to the subject without food, at least about 8 hours prior to the administration and at least about 2 hours post the administration.

In some embodiments, sotorasib is administered once daily, in about 5 minutes after administration of the compound of formula (10b).

### III-6: Efficacy

“A Phase 1 Study of the SHP2 Inhibitor Compound (10b) in Combination with the KRAS G12C Inhibitor Sotorasib in Patients with Advanced or Metastatic Non-Small Cell Lung Cancer with a KRAS G12C Mutation” can evaluate the safety, tolerability, and efficacy of the compound of formula (10b) in combination with sotorasib to reduce or stabilize solid tumors in subjects, as summarized in Example 3.

In various embodiments, the subject is administered the therapy for at least 1 month, at least 2 months, at least 3 months, at least 4 months, at least 5 months, at least 6 months, at least 7 months, at least 8 months, at least 9 months, at least 10 months, at least 11 months, at least 12 months, at least 15 months, at least 18 months, at least 21 months, or at least 23 months, e.g., for 1 month, 2 months, 3 months, 4 months, 5 months, 6 months, 7 months, 8 months, 9 months, 10 months, 11 months, 12 months, 15 months, 18 months, 21 months, or 24 months. In various embodiments, the subject is administered the therapy for at least 1 month. In various embodiments, the subject is administered the therapy for at least 3 months. In various embodiments, the subject is administered the therapy for at least 6 months. In various embodiments, the subject is administered the therapy for at least 8 months.

The subject can respond to the therapy as measured by at least a stable disease (SD), as determined by Response Evaluation Criteria in Solid Tumors (RECIST) 1.1 protocol (Eisenhauer, et al., Eur J Cancer; 2009; 45(2):228-247). RECIST v1.1 is discussed in detail in the examples below. An at least stable disease is one that is a stable disease, has shown a partial response (PR) or has shown a complete response (CR) (i.e., “at least SD”=SD+PR+CR, often referred to as disease control). In various embodiments, the stable disease has neither sufficient shrinkage to qualify for partial response (PR) nor sufficient increase to qualify for progressive disease (PD). In various embodiments, the subject exhibits at least a partial response (i.e., “at least PR”=PR+CR, often referred to as objective response).

Response can be measured by one or more of decrease in tumor size, suppression or decrease of tumor growth, decrease in target or tumor lesions, delayed time to progression, no new tumor or lesion, a decrease in new tumor formation, an increase in survival or progression-free survival (PFS), and no metastases. In various embodiments, the progression of a subject's disease can be assessed by measuring tumor size, tumor lesions, or formation of new tumors or lesions, by assessing the subject using a computerized tomography (CT) scan, a positron emission tomography (PET) scan, a magnetic resonance imaging (MRI) scan, an X-ray, ultrasound, or some combination thereof.

Progression free survival (PFS) can be assessed as described in the RECIST 1.1 protocol. In various embodiments, the subject exhibits a PFS of at least 1 month. In various embodiments, the subject exhibits a PFS of at least 3 months. In some embodiments, the subject exhibits a PFS of at least 6 months.

Administration of a therapeutically effective amount of the compound of formula (I) or (10b) in combination with a therapeutically effective amount of sotorasib can reduce or substantially eliminate cancers or solid tumors in subjects. In some embodiments, the therapeutically effective amount of formula (I) or (10b) in combination with sotorasib substantially eliminates the solid tumor. In some embodiments, the therapeutically effective amount of formula (I) or (10b) in combination with sotorasib reduces a volume of the solid tumor at least about 10%, about 20%, about 30%, about 40%, about 50%, about 60%, about 70%, about 80%, about 90%, or more. In some embodiments, the therapeutically effective amount of formula (I) or (10b) in combination with sotorasib reduces a volume of the solid tumor in a size of from about 10% to about 90%, from about 10% to about 80%, from about 10% to about 70%, from about 10% to about 60%, from about 10% to about 50%, from about 10% to about 40%, from about 10% to about 30%, from about 10% to about 20%, from about 20% to about 90%, from about 20% to about 80%, from about 20% to about 70%, from about 20% to about 60%, from about 20% to about 50%, from about 20% to about 40%, from about 20% to about 30%, from about 30% to about 90%, from about 30% to about 80%, from about 30% to about 70%, from about 30% to about 60%, from about 30% to about 50%, from about 30% to about 40%, from about 40% to about 90%, from about 40% to about 80%, from about 40% to about 70%, from about 40% to about 60%, from about 40% to about 50%, from about 50% to about 90%, from about 50% to about 80%, from about 50% to about 70%, from about 50% to about 60%, from about 60% to about 90%, from about 60% to about 80%, from about 60% to about 70%, from about 70% to about 90%, from about 70% to about 80%, from about 80% to about 90%, or any range therein. In some embodiments, the therapeutically effective amount of formula (I) or (10b) in combination with sotorasib reduces a volume of the solid tumor about 10%, about 20%, about 30%, about 40%, about 50%, about 60%, about 70%, about 80%, or about 90%.

Administration of a therapeutically effective amount of the compound of formula (I) or (10b) in combination with a therapeutically effective amount of sotorasib can stabilize cancers or solid tumors in subjects. In some embodiments, the therapeutically effective amount of formula (I) or (10b) in combination with sotorasib stabilize the solid tumor.

Administration of a therapeutically effective amount of the compound of formula (I) or (10b) in combination with a therapeutically effective amount of sotorasib can maintain a reduction or stabilization of cancers or solid tumors in subjects for a period of time (e.g., 1 to 12 months). In some embodiments, the solid tumor is reduced or stabilized for a period of at least about one month with the therapeutically effective amount of the compound of formula (I) or (10b) in combination with sotorasib. In some embodiments, the solid tumor is reduced or stabilized for a period of at least about 2, 3, 4, 5, 6, 7, 8, 9, 10, 11, or 12 months with the therapeutically effective amount of the compound of formula (10b) in combination with sotorasib. In some embodiments, the solid tumor is reduced or stabilized for a period of from about 1 to about 12 months, from about 1 to about 6 months, from about 1 to about 3 months, or from about 1 to about 2 months.

In some embodiments, the subject is further evaluated to by one or more tests (e.g., as described herein) to provide overall assessments including plasma pharmacokinetic and/or pharmacodynamic profiles. Examples of such tests are described in, e.g., Example 3.

In some embodiments, the subject is further evaluated for one or more biomarkers to determine a correlation of the one or more biomarkers to an antitumor response. Examples of such evaluation are described in Example 3.

### IV. Composition

### Compositions of Compounds of Formula (I) or (10b)

The oral dosage forms including the compound of formula (I) or (10b) can be in any oral dosage forms including one or more pharmaceutically acceptable carriers and/or excipients. Oral preparations include tablets, pills, powder, dragees, capsules, liquids, lozenges, cachets, gels, syrups, slurries, suspensions, etc., suitable for ingestion by the patient.

For preparing oral dosage forms including the compound of formula (I) or (10b), pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired.

The powders, capsules and tablets preferably contain from 5% to 70% of the active pharmaceutical ingredient (e.g., the compound of formula (I) or (10b)) or from about 10% to about 70% of the active pharmaceutical ingredient. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the dosage forms can also be used orally using, for example, push-fit capsules made of gelatin, as well as soft, sealed capsules made of gelatin and a coating such as glycerol or sorbitol. Push-fit capsules can contain the active pharmaceutical ingredient mixed with a filler or binders such as lactose or starches, lubricants such as talc or magnesium stearate, and, optionally, stabilizers. In soft capsules, the active pharmaceutical ingredient may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active pharmaceutical ingredient are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions.

Aqueous solutions suitable for oral use can be prepared by dissolving the active pharmaceutical ingredient in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the active pharmaceutical ingredient in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations including the compound of formula (I) or (10b) can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present disclosure (including the compound of formula (I) or (10b)) can be prepared in a wide variety of oral, parenteral and topical dosage forms. Oral preparations include tablets, pills, powder, dragees, capsules, liquids, lozenges, cachets, gels, syrups, slurries, suspensions, etc., suitable for ingestion by the patient. The compositions of the present disclosure can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present disclosure can be administered transdermally. The compositions of this disclosure can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

For preparing pharmaceutical compositions of the present disclosure (including the compound of formula (I) or (10b)), pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active components are mixed with the carrier having the necessary binding properties in suitable proportions and compacted in a particular shape and size.

The powders, capsules and tablets preferably contain from about 5% to about 70% of the active compounds, such as from about 10% to about 70% of the active compounds (e.g., the compound of formula (I) or (10b)). Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the present disclosure can also be used orally using, for example, push-fit capsules made of gelatin, as well as soft, sealed capsules made of gelatin and a coating such as glycerol or sorbitol. Push-fit capsules can contain the active compounds (e.g., the compound of formula (I) or (10b) and sotorasib) mixed with a filler or binders such as lactose or starches, lubricants such as talc or magnesium stearate, and, optionally, stabilizers. In soft capsules, the active compounds (e.g., the compound of formula (I) or (10b) and sotorasib) may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active compounds (e.g., the compound of formula (I) or (10b)) are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active compounds (e.g., the compound of formula (I) or (10b)), as defined and described herein, in water and adding optional suitable colorants, flavors, stabilizers, and thickening agents. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the active compounds (e.g., the compound of formula (I) or (10b)) in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the present disclosure can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present disclosure (including the compound of formula (I) or (10b)) can be delivered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The compositions of the present disclosure (including the compound of formula (I) or (10b)) can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present disclosure can be formulated for parenteral administration, such as intravenous (IV) administration or administration into a body cavity or lumen of an organ. The formulations for administration will commonly comprise a solution of the compositions of the present disclosure dissolved in a pharmaceutically acceptable carrier. Among the acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by various sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present disclosure in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present disclosure can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present disclosure into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

Lipid-based drug delivery systems include lipid solutions, lipid emulsions, lipid dispersions, self-emulsifying drug delivery systems (SEDDS) and self-microemulsifying drug delivery systems (SMEDDS). In particular, SEDDS and SMEDDS are isotropic mixtures of lipids, surfactants and co-surfactants that can disperse spontaneously in aqueous media and form fine emulsions (SEDDS) or microemulsions (SMEDDS). Lipids useful in the formulations of the present disclosure include any natural or synthetic lipids including, but not limited to, sesame seed oil, olive oil, castor oil, peanut oil, fatty acid esters, glycerol esters, Labrafil®, Labrasol®, Cremophor®, Solutol®, Tween®, Capryol®, Capmul®, Captex®, and Peceol®.

The pharmaceutical formulations of the present disclosure can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in, e.g., 1 mM-50 mM histidine, 0.1%-2% sucrose, 2%-7% mannitol at a pH range of 4.5 to 5.5, that is combined with buffer prior to use.

The pharmaceutical formulations of the present disclosure can be provided as a salt and can be formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

### Compositions of Sotorasib

In some embodiments, sotorasib is provided in a tablet comprising sotorasib, microcrystalline cellulose, lactose monohydrate, croscarmellose sodium, and magnesium stearate. In some embodiments, the tablets are film-coated. In some embodiments, the film coating material comprises polyvinyl alcohol, titanium dioxide, polyethylene glycol, talc, and iron oxide yellow.

### V. Kits

In another aspect, the present disclosure provides a kit for treating cancer in a subject, the kit including:
a) a therapeutically effective amount of a compound represented by formula (I); and
b) a therapeutically effective amount of sotorasib, together with instruction for effective administration,
wherein the compound of formula (I) is as defined and described herein.

The cancer and/or solid tumor are described according to Section III-2: Cancer/Solid Tumor. In some embodiments, the cancer and/or solid tumor are any of embodiments as described in Section III-2: Cancer/Solid Tumor.

The subject is described according to Section III-3: Subject. In some embodiments, the subject is any of embodiments as described in Section III-3: Subject.

The compound of formula (I) is described according to Section III-1: Compound of Formula (I). In some embodiments, the compound of formula (I) is any of embodiments as described in Section III-1: Compound of Formula (I). In some embodiments, the PTPN11 inhibitor of formula (I) is the compound of formula (10b).

In some embodiments, the kit includes instructions for administration of the compound of formula (I) or (10b) and sotorasib. In some embodiments, the kit includes instructions for administration of the compound of formula (10b) and sotorasib. In some embodiments, such instructions include directions relating to safety provisions as well as timing and amounts of administration of the compound of formula (I) or (10b) and sotorasib. In some embodiments, such instructions include directions relating to safety provisions as well as timing and amounts of administration of the compound of formula (10b) and sotorasib.

The compound of formula (I) or (10b) as described herein and sotorasib can be formulated for concomitant administration or sequential administration. In some embodiments, the compound of formula (I) or (10b) and sotorasib are formulated for concomitant administration. In some embodiments, the compound of formula (I) or (10b) and sotorasib are formulated for sequential administration. In some embodiments, the compound of formula (I) or (10b) is administered prior to the administration of sotorasib. In some embodiments, the compound of formula (I) or (10b) is administered after the administration of sotorasib.

### VI. Embodiments

Embodiment 1. A method of treating cancer in a subject, comprising administering to the subject:
a) a therapeutically effective amount of a compound represented by formula (I):

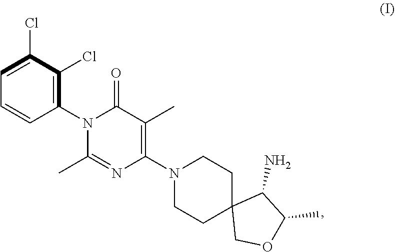

or a pharmaceutically acceptable salt, hydrate, solvate, stereoisomer, conformational isomer, tautomer, or a combination thereof; and
b) a therapeutically effective amount of sotorasib (AMG 510).
Embodiment 2. The method of embodiment 1, wherein the compound of formula (I) is represented by formula (10b):

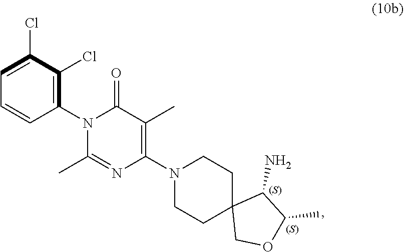

having the name of 6-((3S,4S)-4-amino-3-methyl-2-oxa-8-azaspiro[4.5]decan-8-yl)-3-(Ra)-(2,3-dichlorophenyl)-2,5-dimethylpyrimidin-4(3H)-one.
Embodiment 3. The method of embodiment 1 or 2, wherein the cancer is characterized by a KRAS mutation.
Embodiment 4. The method of embodiment 3, wherein the cancer is characterized by a KRAS G12C mutation.
Embodiment 5. The method of any one of embodiments 1 to 4, wherein the cancer comprises a solid tumor.
Embodiment 6. The method of any one of embodiments 1 to 5, wherein the cancer is lung cancer, colorectal cancer, pancreatic cancer, urothelial carcinoma, stomach cancer, mesothelioma, or a combination thereof.
Embodiment 7. The method of embodiment 6, wherein the cancer is non-small cell lung cancer (NSCLC).
Embodiment 8. The method of any one of embodiments 1 to 7, wherein the cancer is a KRAS G12C-positive cancer resistant to a KRAS G12C inhibitor.
Embodiment 9. The method of any one of embodiments 1 to 8, wherein the cancer is a KRAS G12C-positive cancer characterized by intrinsic and/or acquired resistance to a KRAS G12C inhibitor.
Embodiment 10. The method of any one of embodiments 1 to 9, wherein the cancer is a KRAS G12C-positive cancer resistant to sotorasib.
Embodiment 11. The method of any one of embodiments 1 to 10, wherein the cancer has progressed or recurred on or after at least one prior line of a systemic therapy comprising a platinum-based doublet chemotherapy and/or an anti-PD-1/PD-L1 therapy, each of which is given in monotherapy or both of which are given in combination therapy.
Embodiment 12. The method of any one of embodiments 1 to 11, wherein the subject does not have an activating mutation in BRAF V600X, PTPN11 (SHP2), or KRAS Q61X.
Embodiment 13. The method of any one of embodiments 1 to 12, wherein the subject is not previously treated with a PTPN11 inhibitor.
Embodiment 14. The method of any one of embodiments 1 to 12, wherein the subject is previously treated with a PTPN11 inhibitor other than a compound of formula (I).
Embodiment 15. The method of any one of embodiments 1 to 12, wherein the subject is previously treated with a compound of formula (I).
Embodiment 16. The method of any one of embodiments 1 to 8 and 10 to 15, wherein the subject is not previously treated with a KRAS G12C inhibitor.
Embodiment 17. The method of any one of embodiments 1 to 15, wherein the subject is previously treated with a KRAS G12C inhibitor.
Embodiment 18. The method of any one of embodiments 1 to 15, wherein the subject is previously treated with sotorasib.
Embodiment 19. The method of any one of embodiments 1 to 18, wherein the subject meets all of inclusion criteria of 1) to 11) according to Example 3, provided that the subject does not meet any one of exclusion criteria of 1) to 17) according to Example 3.
Embodiment 20. The method of any one of embodiments 1 to 19, wherein the subject is human.
Embodiment 21. The method of any one of embodiments 1 to 20, wherein the compound of formula (I) or (10b) and sotorasib are administered concomitantly.
Embodiment 22. The method of any one of embodiments 1 to 20, wherein the compound of formula (I) or (10b) and sotorasib are administered sequentially.
Embodiment 23. The method of embodiment 22, wherein the compound of formula (I) or (10b) is administered prior to the administration of sotorasib.
Embodiment 24. The method of embodiment 22, wherein the compound of formula (I) or (10b) is administered after the administration of sotorasib.
Embodiment 25. The method of any one of embodiments 1 to 24, wherein the compound of formula (I) or (10b) or sotorasib is administered orally, or each of sotorasib and the compound of formula (I) or (10b) are administered orally.
Embodiment 26. The method of any one of embodiments 1 to 25, wherein the compound of formula (I) or (10b) is administered orally.
Embodiment 27. The method of any one of embodiments 1 to 26, wherein sotorasib is administered orally.
Embodiment 28. The method of any one of embodiments 1 to 27, wherein the compound of formula (I) or (10b) and sotorasib are provided in jointly therapeutically effective amounts.
Embodiment 29. The method of any one of embodiments 1 to 27, wherein the compound of formula (I) or (10b) and sotorasib are provided in synergistically effective amounts.
Embodiment 30. The method of any one of embodiments 1 to 29, wherein the compound of formula (I) or (10b) and/or sotorasib is used at a dose different than when it is used alone.
Embodiment 31. The method of embodiment 30, wherein the compound of formula (I) or (10b) is used at a dose lower than when it is used alone.
Embodiment 32. The method of embodiment 30, wherein the compound of formula (I) or (10b) is used at a dose higher than when it is used alone.
Embodiment 33. The method of any one of embodiments 30-32, wherein sotorasib is used at a dose lower than when it is used alone.
Embodiment 34. The method of any one of embodiments 30-32, wherein sotorasib is used at a dose higher than when it is used alone.
Embodiment 35. The method of any one of embodiments 1 to 34, wherein the treating comprises one or more treatment cycles; each of one or more treatment cycles has a duration of about 28 days; and the compound of formula (I) or (10b) and/or sotorasib are administered daily.
Embodiment 36. The method of any one of embodiments 1 to 35, wherein the administration of the compound of formula (I) or (10b) and sotorasib comprises one or more dose escalations, dose retentions, or dose de-escalations of the compound of formula (I) or (10b) and/or sotorasib.
Embodiment 37. The method of embodiment 36, wherein the administration of the compound of formula (I) or (10b) and sotorasib comprises one or more dose escalations, dose retentions, or dose de-escalations of the compound of formula (I) or (10b).
Embodiment 38. The method of embodiment 37, wherein the one or more dose escalations, dose retentions, or dose de-escalations of the compound of formula (I) or (10b) are determined by a dose-limiting toxicity (DLT) assessment.
Embodiment 39. The method of embodiment 38, wherein the administration of the compound of formula (I) or (10b) comprises a dose escalation after a previous treatment cycle, when a dose-limiting toxicity (DLT) rate is less than about 19.7% as determined by a DLT assessment.
Embodiment 40. The method of embodiment 38, wherein the administration of the compound of formula (I) or (10b) comprises a dose de-escalation after a previous treatment cycle, when a dose-limiting toxicity rate is more than about 29.8% as determined by a DLT assessment.
Embodiment 41. The method of embodiment 38, wherein the administration of the compound of formula (I) or (10b) comprises a dose retention after a previous treatment cycle, when a dose-limiting toxicity rate is in a range of from about 21.9% to about 29.8% as determined by a DLT assessment.
Embodiment 42. The method of any one of embodiments 35 to 41, wherein the treating comprises a dose escalation period, and wherein, after the dose escalation period, the treating further comprises a dose expansion/optimization period; and the compound of formula (I) or (10b) is administered at a dose regimen determined during the dose escalation period.
Embodiment 43. The method of embodiment 42, wherein, during the dose expansion/optimization period, the administration of the compound of formula (I) or (10b) comprises one or more dose adjustments.
Embodiment 44. The method of any one of embodiments 1 to 43, wherein the therapeutically effective amount of sotorasib is a total daily dosage of about 120 mg, about 240 mg, about 360 mg, about 480 mg, about 600 mg, about 720 mg, about 840 mg, or about 960 mg.
Embodiment 45. The method of embodiment 44, wherein the therapeutically effective amount of sotorasib is a total daily dosage of about 960 mg.
Embodiment 46. The method of any one of embodiments 1 to 45, wherein the therapeutically effective amount of the compound of formula (I) or (10b) is a total daily dosage of from about 100 mg to about 2000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 100 mg to about 700 mg, from about 150 mg to about 700 mg, from about 200 mg to about 700 mg, from about 250 mg to about 700 mg, from about 300 mg to about 700 mg, from about 3 mg to about 700 mg, from about 400 mg to about 700 mg, from about 450 mg to about 700 mg, from about 500 mg to about 700 mg, from about 550 mg to about 700 mg, from about 100 mg to about 550 mg, from about 150 mg to about 550 mg, from about 200 mg to about 550 mg, from about 250 mg to about 550 mg, from about 300 mg to about 550 mg, from about 350 mg to about 550 mg, from about 400 mg to about 550 mg, from about 450 mg to about 550 mg, from about 100 mg to about 400 mg, from about 150 mg to about 400 mg, from about 200 mg to about 400 mg, from about 250 mg to about 400 mg, or from about 300 mg to about 400 mg, on a salt-free and anhydrous basis.
Embodiment 47. The method of embodiment 46, wherein the therapeutically effective amount is a total daily dosage of from about 250 mg to about 400 mg, from about 400 mg to about 550 mg, or from about 550 mg to about 700 mg of the compound of formula (I) or (10b), on a salt-free and anhydrous basis.
Embodiment 48. The method of embodiment 47, wherein the therapeutically effective amount is a total daily dosage of about 250 mg, about 400 mg, or about 550 mg of the compound of formula (I) or (10b), on a salt-free and anhydrous basis.
Embodiment 49. The method of any one of embodiments 46 to 48, wherein the therapeutically effective amount is a total daily dosage of about 250 mg of the compound of formula (I) or (10b), on a salt-free and anhydrous basis.
Embodiment 50. The method of any one of embodiments 46 to 48, wherein the therapeutically effective amount is a total daily dosage of about 400 mg of the compound of formula (I) or (10b), on a salt-free and anhydrous basis.
Embodiment 51. The method of any one of embodiments 46 to 48, wherein the therapeutically effective amount is a total daily dosage of about 550 mg of the compound of formula (I) or (10b), on a salt-free and anhydrous basis.
Embodiment 52. The method of any one of embodiments 1 to 51, wherein the compound of formula (10b) and sotorasib are each administered orally.
Embodiment 53. The method of any one of embodiments 1 to 52, wherein the compound of formula (10b) is administered once, twice, three times, or four times daily.
Embodiment 54. The method of embodiment 53, wherein the compound of formula (I) or (10b) is administered once daily; and sotorasib is administered once daily.
Embodiment 55. The method of any one of embodiments 1 to 54, wherein the compound of formula (I) or (10b) is provided in a tablet formulation.
Embodiment 56. The method of any one of embodiments 1 to 55, wherein the treating reduces a volume of the cancer or a solid tumor at least about 10%, about 20%, about 30%, about 40%, about 50%, about 60%, about 70%, about 80%, or about 90%.
Embodiment 57. The method of any one of embodiments 1 to 55, wherein the treating stabilizes the cancer or a solid tumor.
Embodiment 58. The method of any one of embodiments 1 to 57, wherein the subject is further evaluated for one or more biomarkers that correlate to an antitumor response.
Embodiment 59. A kit for treating cancer in a subject, comprising:
a) a therapeutically effective amount of a compound represented by formula (I):

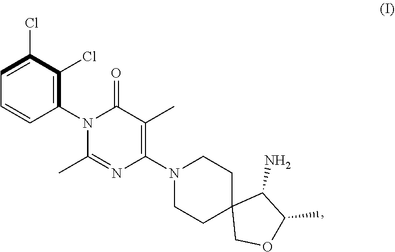

or a pharmaceutically acceptable salt, hydrate, solvate, stereoisomer, conformational isomer, tautomer, or a combination thereof, or a compound represented by formula (10b):

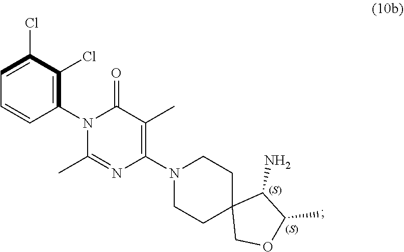

and
b) a therapeutically effective amount of sotorasib, together with instruction for effective administration.
Embodiment 60. The kit of embodiment 59, wherein the compound of formula (I) or (10b) and sotorasib are formulated for concomitant administration.
Embodiment 61. The kit of embodiment 59, wherein the compound of formula (I) or (10b) and sotorasib are formulated for sequential administration.

VII. LIST OF ABBREVIATIONS

| Abbreviation | Definition |
|---|---|
| (e)CRF | (electronic) case report form |
| (p)ERK | (phosphorylated) extracellular signal-regulated kinase |
| AAALAC | Association for the Assessment and Accreditation for |
| | Laboratory Animal Care |
| AE | adverse event |
| ALK | anaplastic lymphoma kinase |
| ALT | alanine aminotransferase (or transaminase) |
| ANC | absolute neutrophil count |
| aPTT | activated partial thromboplastin time |
| AST | aspartate aminotransferase (or transaminase) |
| ATCC | American Type Culture Collection |
| AUC | area under the plasma concentration-time curve |
| BBP | BridgeBio Pharma |
| BCRP | breast cancer resistance protein |
| BICR | blinded independent central review |
| BOIN | Bayesian optimal interval |
| BQL | Below Quantitation Limit |
| BW | Body Weight |
| BWL | Body Weight Loss |
| CI | confidence interval |
| CLIA | Clinical Laboratory Improvement Amendments |
| Cₘₐₓ | maximum plasma drug concentration |
| CNS | central nervous system |
| CR | complete response(s) |
| CRO | contract research organization |
| CT | computed tomography |
| CTCAE | Common Terminology Criteria for Adverse Events |
| ctDNA | circulating tumor deoxyribonucleic acid |
| CYP | cytochrome P450 |
| DLT | dose-limiting toxicity |
| DNA | deoxyribonucleic acid |
| DOR | duration of response |
| DUSP6 | dual specificity phosphatase 6 |
| ECG | electrocardiogram |
| ECHO | echocardiogram |
| ECOG | Eastern Cooperative Oncology Group |
| EDC | electronic data capture (system) |
| EDTA | Ethylenediaminetetraacetic acid |
| EGFR | epidermal growth factor receptor |
| EORTC | European Organization for Research and Treatment |
| | of Cancer |
| EOS | end of study |
| EOT | end of treatment |
| FBS | fetal bovine serum |
| FDA | Food and Drug Administration |
| FSH | follicle-stimulating hormone |
| G12C | Glycine 12 to cysteine mutation |
| GCP | Good Clinical Practice |
| GLP | Good Laboratory Practice |
| GMP | Good Manufacturing Practice |
| GTP | guanosine triphosphate |
| HBV | hepatitis B virus |
| HCV | hepatitis C virus |
| HIV | human immunodeficiency virus |
| HNSTD | highest non-severely toxic dose |
| HPMC | Hydroxypropyl methylcellulose |
| IACS | Institute for Applied Cancer Science |
| IACUC | Institutional Animal Care and Use Committee |
| IB | Investigator's Brochure |
| ICF | informed consent form |
| ICH | International Conference on Harmonization |
| IEC | Independent Ethics Committee |
| INR | international normalized ratio |
| IRB | Institutional Review Board |
| IV | intravenous |
| KRAS | Kirsten rat sarcoma viral oncogene homolog |
| LC-MS/MS | liquid chromatography with tandem mass |
| | spectrometry |
| LD (or LDi) | longest diameter |
| LVEF | left ventricular ejection fraction |
| MAPK | mitogen-activated protein kinase |
| MATE | multidrug and toxin extrusion protein |
| MRI | magnetic resonance imaging |
| MTD | maximum tolerated dose |
| MUGA | multiple-gated acquisition scan (radionuclide |
| | angiography) |
| NCI | National Cancer Institute |
| NGS | next generation sequencing |
| NOD/SCID | Nonobese Diabetic/Severe Combined |
| | Immunodeficiency |
| NSCLC | non-small cell lung cancer |
| OATP | organic anion transporter polypeptide |
| ORR | objective response rate |
| OS | overall survival |
| PBS | Phosphate-buffered saline |
| PD | progressive disease |
| PD-1 | programmed cell death-1 |
| PD-L1 | programmed cell death ligand-1 |
| PET | positron emission tomography |
| PFS | progression free survival |
| P-gp | P-glycoprotein |
| PI | package insert |
| PK | pharmacokinetic |
| PO | Per os (oral administration, by mouth) |
| PR | partial response |
| PS | performance status |
| PT | prothrombin time |
| PTPN11 | protein tyrosine phosphatase non-receptor type 11, |
| | the gene encoding SHP2 |
| QD | once daily |
| RAF | rapidly accelerated fibrosarcoma |
| RAS | any of a family of genes that undergo mutation to |
| | oncogenes and especially to some commonly linked |
| | to human cancers (as of the colon, lung, and pancreas) |
| REB | Research Ethics Board |
| RECIST | Response Evaluation Criteria in Solid Tumors |
| RNA | ribonucleic acid |
| RP1bD | recommended phase 1b dose |
| RP2D | recommended phase 2 dose |
| RPM | Round per minute |
| RPMI | Roswell Park Memorial Institute |
| RTK | receptor tyrosine kinase |
| SAE | serious adverse event |
| SAP | Statistical Analysis Plan |
| SD (Example 1) | Standard Deviation |
| SD | stable disease |
| SEM | Standard error of the mean |
| SGOT | serum glutamic-oxaloacetic transaminase |
| SGPT | serum glutamic-pyruvic transaminase |
| SHP2 | Src homology-2-containing protein tyrosine |
| | phosphatase 2 |
| SRC | Safety Review Committee |
| STD10 | severely toxic dose in 10% of animals |
| SUSAR | suspected unexpected serious adverse reaction |
| TEAE | treatment-emergent adverse event |
| TGI | Tumor Growth Inhibition |
| Tₘₐₓ | time to reach maximum drug concentration |
| | following drug administration |
| UHPLC | Ultra High Performance Liquid Chromatography |
| ULN | upper limit of normal |
| US | United States |

### VIII. Examples

### Example 1: In Vivo Efficacy of Formula (10b) and Compound A

### A. Materials

### Test Article #1—Formula (10b):

Chemical name: 6-[(3S,4S)-4-amino-3-methyl-2-oxa-8-azaspiro [4.5]decan-8-yl]-3-(2,3-dichlorophenyl)-2,5-dimethyl-3,4-dihydropyrimidin-4-one

Molecular formula: C₂₁H₂₆Cl₂N₄O₂.

Molecular weight: 437.37

### Test Article #2—Compound A:

Chemical name: 6-fluoro-7-(2-fluoro-6-hydroxyphenyl)-1-[4-methyl-2-(propan-2-yl)pyridin-3-yl]-4-[(2S)-2-methyl-4-(prop-2-enoyl)piperazin-1-yl]pyrido[2,3-d]pyrimidin-2(1H)-one
CAS #: 252403-56-6
Molecular formula: C₃₀H₃₀F₂N₆O₃
Molecular weight: 560.6

Cell lines. The NCI-H358 cell line was purchased from ATCC. It is a human NSCLC cell line, harboring heterozygous mutation of KRAS G12C. The cells were cultured in RPMI 1640 containing glutamine (Thermo Fisher #22400-089)+10% fetal bovine serum (FBS, Thermo Fisher #10099-141) in 37° C. tissue culture incubator (Thermo Fisher) with 5% CO₂.

Test animals. Female NOD/SCID mice (Beijing Anikeeper Biotech Co., Ltd) were utilized in this experiment. Animals were 6˜8 weeks of age at the time of xenograft implantation. The protocol and any amendment(s) or procedures involving the care and use of animals in this study was reviewed and approved by the Institutional Animal Care and Use Committee (IACUC) of Crown Bioscience Ltd. prior to execution. During the study, the care and use of animals was conducted in accordance with the regulations of the Association for Assessment and Accreditation of Laboratory Animal Care (AAALAC).

All mice were housed in AAALAC accredited animal research facilities at the Crown Bioscience Beijing, at Light Muller Building, Changping Sector of Zhongguancun Scientific Park, No. 21 Huoju Road, Changping District, Beijing, China. All animals were maintained under the supervision and care of Veterinary of the Animal Facilities, who oversee a comprehensive and well-executed health surveillance program.

Mice were housed in polysulfone IVC cage (325 mm×210 mm×180 mm), with a maximum of five animals and had free access to food and water with light 12-hour on/12-hour off. All animals received irradiated Standard rodent chow—Rats & Mice Growing and Breeding Feed diet (Beijing Keao Xieli Feed Co., LTD) ad libitum. Mice were monitored daily and cages changed once every second week.

### B. Experimental Procedures

Formulation. Formula (10b) was prepared in 0.5% methylcellulose. To prepare 0.5% methylcellulose solution, methylcellulose powder (400 cP, Sigma #M0262) was added to heated 80° C. sterile water. The solution was incubated at 80° C. with stirring for 3˜4 hours, then incubated at 4° C. with continuous stirring for 18 hours. After adjusting the final volume with sterile H₂O, the solution was stirred for another 30 min at 4° C., then filtered using 0.45 μM sterile filter. The prepared 0.5% methylcellulose solution was stored at 4° C. for future use.

To prepare formula (10b) dosing suspension in 0.5% methylcellulose, the weighed compound was placed in a glass vial and 0.5% methylcellulose solution was added to the vial with a syringe. After vortexing for 30 seconds, the vial was sonicated in a water bath sonicator (Shanghai Kudos Ultrasonic Instrument Co., LTD, Model SK2510HP) on “High” setting at room temperature for ˜20 min until an off-white suspension without visible solids was obtained. The prepared dosing suspension was stored at 4° C. with gentle continuous stirring. Fresh dosing suspension was prepared once a week.

Compound A was prepared in Labrasol (Gattefosse #3074). To prepare the Compound A dosing solution, the weighed compound was placed in a glass vial and Labrasol was added to the vial. The mixture was vortexed for 30 sec, sonicated in a water bath sonicator (Shanghai Kudos Ultrasonic Instrument Co., LTD, Model SK2510HP) for ˜5 min until a pale yellow solution was obtained. The dosing solution was stored at 4° C. with gentle continuous stirring. Fresh dosing solution was prepared once a week. Immediately before dosing the animals with Compound A, the dosing solution was mixed with an equal volume of sterile ddH₂O, vortexed, and then incubated at room temperature for 10 min, at which time a uniform solution was formed and was used to dose the mice. The diluted dosing solution was used within one hour following addition of water.

In vivo modeling, treatment and data analysis. For xenograft studies with the NCI-H358 cells, 5 million cells with 50% Matrigel (BD bioscience #356234) in sterile PBS (Hyclone Laboratories #SH30256.01) in a volume of 100 μL were injected subcutaneously into the right flank of female 6˜8 weeks old NOD/SCID mice (Beijing Anikeeper Biotech Co., Ltd). Tumor size measurement was performed with caliper and calculated using a standard formula: length×width²/2, where length and width were the long and short diameters of the tumor, respectively.

When the average tumor volume reached 250 mm³, mice were randomized into groups of 8 based on both tumor volume and body weight. Randomization was performed based on the “Matched distribution” method/“Stratified” method in the StudyDirector software version 3.1.399.19 (StudyLog). The mice were then treated with vehicle, Compound A alone at one of three dosing levels (100 mg/kg QD, 30 mg/kg QD and 10 mg/kg QD), formula (10b) alone at one of two dosing levels (100 mg/kg QD and 50 mg/kg QD), or the combination of the two compounds, through oral gavage (PO), as shown below. Formula (10b) was administered in the morning, and Compound A was administered in the afternoon, with the morning and afternoon dosing separated by 6 hours.

| Group | Treatment |
|---|---|
| A | 0.5% methylcellulose QD AM + 50% Labrasol QD PM |
| B | 0.5% methylcellulose AM + Compound A 100 mg/kg QD PM |
| C | 0.5% methylcellulose AM + Compound A 30 mg/kg QD PM |
| D | 0.5% methylcellulose AM + Compound A 10 mg/kg QD PM |
| E | Formula (10b) 100 mg/kg QD AM + 50% Labrasol QD PM |
| F | Formula (10b) 50 mg/kg QD AM + 50% Labrasol QD PM |
| G | Formula (10b) 100 mg/kg QD AM + Compound A 100 mg/kg |
| | QD PM |
| H | Formula (10b) 50 mg/kg QD AM + Compound A 100 mg/kg |
| | QD PM |
| I | Formula (10b) 100 mg/kg QD AM + Compound A 30 mg/kg |
| | QD PM |
| J | Formula (10b) 100 mg/kg QD AM + Compound A 10 mg/kg |
| | QD PM |

Dosing started ˜2 weeks post the subcutaneous implantation of the tumor cells. Dosing volume for formula (10b) was 5 mL/kg, for Compound A was 6 mL/kg, and was adjusted based on individual mouse weight from the biweekly measurements. Tumor volume was measured twice a week. The body weight and tumor volume were captured in the StudyDirector software version 3.1.399.19 (StudyLog). After dosing for 21 days (all dosing levels but 10 mg/kg formula (10b)) or 29 days (10 mg/kg formula (10b)), plasma samples were harvested 2 hours after the final dose of Compound A (8 hours after the final dose of formula (10b)), following the blood collection method described below.

Data were analyzed using Microsoft Excel and GraphPad Prism 8.0. Day 0 was the day before treatment started. Body weight change and tumor growth inhibition (TGI) were calculated based on the following formulas.

$$Body Weight Change \% = ( {BW}_{i} - {BW}_{0} ) / {BW}_{0} * 100 \%$$

BWᵢ and BW₀ are the body weight of an individual mouse on measurement day I and on day 0, respectively.

$$TGI \% = ( {C}_{i} - {T}_{i} ) - ( {C}_{i} - {C}_{0} ) * 100 \%$$

Tᵢ and Cᵢ are the mean tumor volumes of the treatment and vehicle groups on the measurement day, respectively; C₀ is the mean tumor volume of the vehicle group on day 0.

Blood collection for pharmacokinetic analysis. Blood from live animals for pharmacokinetic (PK) analyses was obtained via the retro-orbital sinus. The mouse was grasped firmly behind the ears and rotated slightly to one side so that a disposable, sterile microcapillary tube could be inserted behind the eyeball and gently rotated to break the sinus cavity. Blood flowed through the capillary tube, directly into a K₂ EDTA tube (Jiangsu KangJian Medical Apparatus Corporation #044022) to a volume of 100 μL. Blood in the tube was inverted several times to distribute the EDTA evenly. To separate plasma, tubes were centrifuged in a bench top centrifuge (Eppendorf, Model 5424R) for 5 minutes at 8000 rpm without braking. The supernatant (plasma) was carefully transferred to a microcentrifuge tube and placed onto dry ice prior to storage at −80° C.

LC-MS/MS quantitation of formula (10b) and Compound A in mouse plasma. Concentrations of formula (10b) and Compound A in mouse plasma were quantitated using a validated LC-MS/MS method. For plasma sample analysis, 20 μL of each sample was precipitated with 200 μL of acetonitrile containing Glipizide (100 ng/mL) as an internal standard. This suspension was vortexed for 1 min and centrifuged at 4,000 rpm for 10 min, after which 80 μL of the extract was aliquoted for LC-MS/MS analysis. LC-MS/MS analysis was conducted on a Shimadzu Nexera UHPLC system coupled with a Sciex 6500 TQ-S triple quadrupole mass spectrometer (MS/MS) operated at the positive mode (ESI+). The mass spec source conditions were set as the following: Ion spray voltage (5500 volts), CAD (8), CUR (35), TEM (450), GS1 (60), GS2 (60), EP (10), CXP (12), CEM (2000) and for formula (10b): DP (40), CE (65), for Compound A: DP (40), CE (47), for the internal standard: DP (35), CE (20). formula (10b) and Compound A were separated using a Waters X-Bridge BEH C18 column (2.1×50 mm, 1.7 μm) and detected by a multiple reaction monitoring transition (m/z 437.10>186.10 for formula (10b), m/z 561.20>134.20 for Compound A and m/z 446.20>321.10 for the internal standard). The injection volume was 2 μL. The LC mobile phase A was 0.025% formic acid-water containing 1 mM ammonium acetate and B was 100% methanol containing 5 mM ammonium acetate. The gradient (% B) was 2% (0-0.4 min), 2-65% (0.4-1.7 min), 65-90% (0.7 to 1.3 min), 90% (1.3-1.9 min), 90-2% (1.9 to 1.91 min), 2% (1.91-2.5 min). The flow rate was 0.6 mL/min. The column temperature was 60° C. Under these conditions, the retention time was 1.42 min for formula (10b), 1.44 min for Compound A and the internal standard. The method was validated with the analytical range of 3-3,000 nM for both formula (10b) and Compound A in untreated CD-1 mouse plasma.

### C. Results

Sotorasib (AMG 510) is a KRAS covalent inhibitor that specifically targets the KRAS G12C mutant. It has demonstrated single agent response in patients with solid tumors, including NSCLC, harboring KRAS G12C mutation. Specifically, in the CodeBreaK 100 study, the subgroup with NSCLC had a confirmed objective response rate (complete or partial response) of 32.2% and a disease control rate (objective response or stable disease) of 88.1%; the median progression-free survival was 6.3 months. Hong et al., N. Engl. J. Med. 2020; 383:1207-1217. Unbiased functional genomics approaches have identified SHP2 as a vulnerability under KRAS G12C inhibition. Inhibition of KRAS G12C induces adaptive feedback activation of the MAPK pathway through multiple RTKs, which signal through SHP2. This study was designed to assess the effect of the SHP2 inhibitor formula (10b) in combination with the KRAS G12C inhibitor Compound A in a human NSCLC tumor model harboring KRAS G12C in vivo.

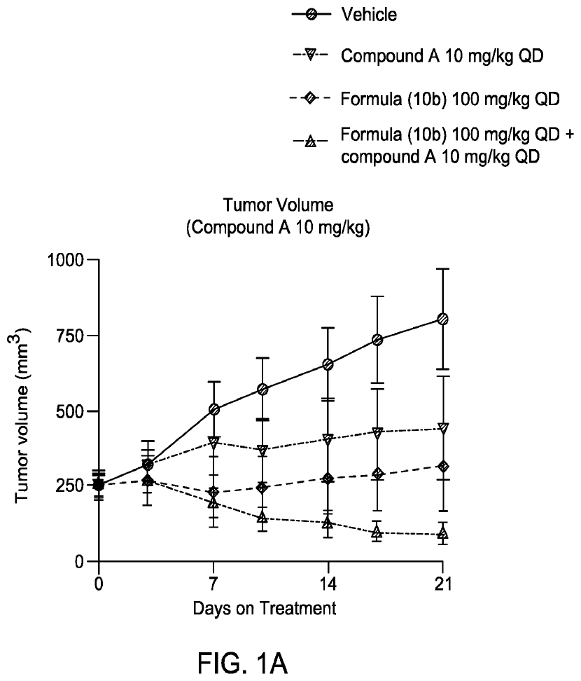

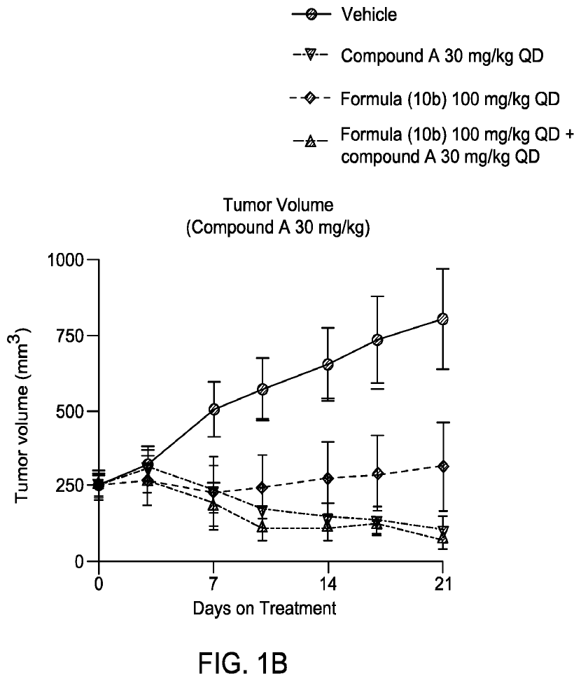

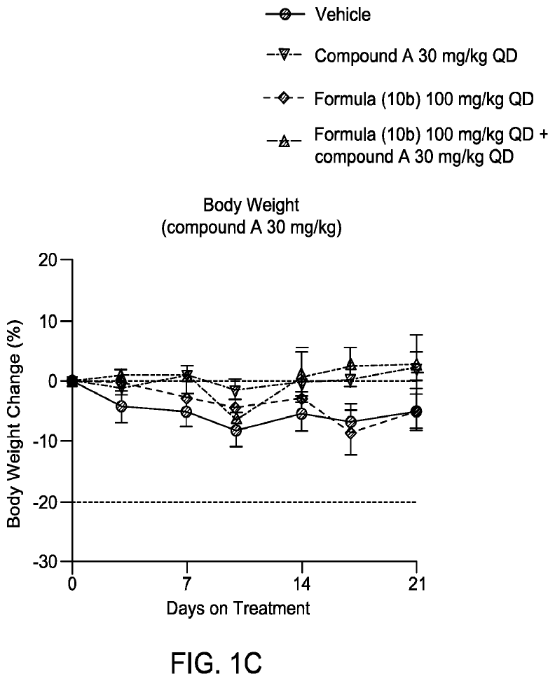

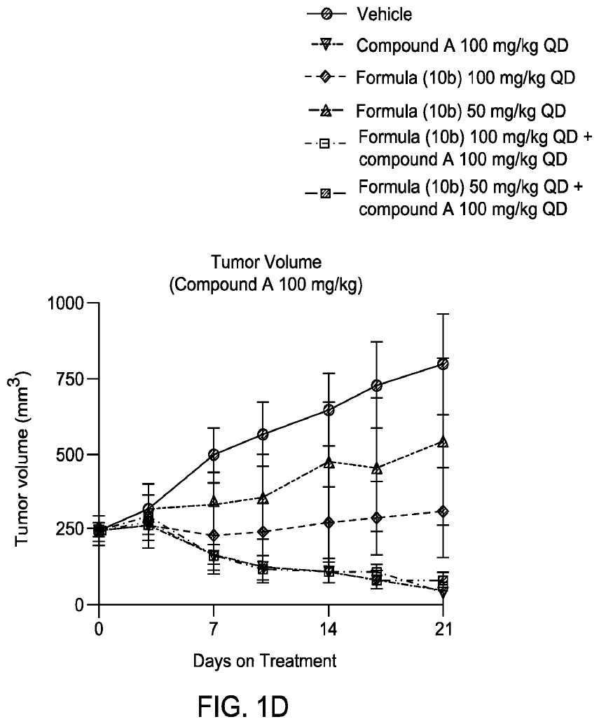

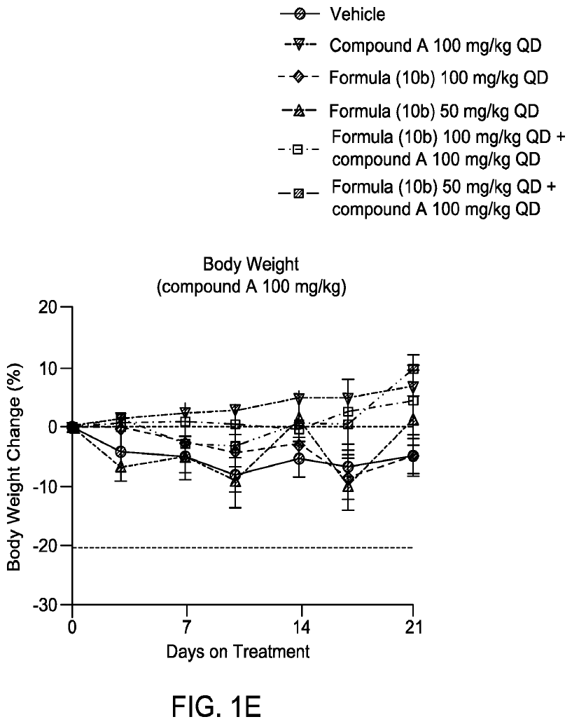

Treatment with the combination of formula (10b) and Compound A resulted in tumor growth inhibition in a subcutaneous model of NSCLC harboring KRAS G12C. A mouse tumor xenograft model was developed by subcutaneous implantation of the NCI-H358 cells which harbor KRAS G12C, and was used to test the anti-tumor response of formula (10b) in combination with Compound A in vivo. Mice harboring established NCI-H358 subcutaneous tumors were randomized and treated with vehicle, Compound A alone, formula (10b) alone, or the combination of the two compounds, delivered orally for 21 days. Formula (10b) was administered every day in the morning, and Compound A was administered every day in the afternoon, with the morning and afternoon dosing separated by 6 hours. Tumor volume was monitored bi-weekly by caliper and body weights were recorded. Data presented in FIGS. 1A-1E represent mean±SEM, and N=8 mice/group for FIGS. 1B-1E and N=10 mice/group for FIG. 1A.

FIG. 1A shows mean (+/−SEM) tumor volume (mm³) of NCI-H358 tumor-bearing female NOD/SCID mice following daily oral dosing of either formula (10b), Compound A, or the combination of both test articles at the indicated dose levels from day 1 to 28.

As shown in FIGS. 1A, 1B, and 1D and Tables 1 and 2, treatment with formula (10b) alone suppressed the growth of the NCI-H358 tumors in a dose-dependent manner, leading to tumor growth inhibition (TGI) of 89% and 46% on study day 21 at 100 mg/kg QD and 50 mg/kg QD, respectively. Treatment with Compound A alone also suppressed the growth of the NCI-H358 tumors in a dose-dependent manner, with tumor regression observed at 100 mg/kg QD, 30 mg/kg QD, and 10 mg/kg QD. Notably, the combination of Compound A 10 mg/kg QD and formula (10b) 100 mg/kg QD more potently suppressed tumor growth as compared to either agent alone, causing tumor regression (FIG. 1A). Notable enhancement of anti-tumor activity was observed when formula (10b) 100 mg/kg QD was combined with Compound A at either 30 mg/kg and 100 mg/kg QD (FIGS. 1B and 1D), likely due to the robust single agent response from Compound A. All dosing conditions were considered well-tolerated, as shown by the maintenance of body weight of the mice during the study (FIGS. 1C and 1E). Although random mouse loss was observed in this study, the mouse loss was not considered treatment related due to lack of dose dependency (Table 3).

TABLE 1
Overview of combination study of formula (10b) at
100 mg/kg and Compound A at 10 mg/kg.

| | Day 29 | | |
|---|---|---|---|
| | Mean tumor | Number of | Mean body |
| Group (n = 10) | regression | regressions | weight change |
| Vehicle | — | 0/10 | +5.1% |
| Formula (10b) 100 mg/kg QD | 14% | 8/10 | −6.5% |
| Compound A 10 mg/kg QD | 13% | 9/10 | −0.3% |
| Compound A 10 mg/kg QD + | 83% | 10/10 | −2.4% |
| Formula (10b) 100 mg/kg QD | | | |

TABLE 2
Endpoint TGI and plasma concentration at 2 hours after the
final dose of Compound A (8 hours after the final dose of
formula (10b)) from the study shown in FIGS. 1B-1E.

| | | Com- | Formula |
|---|---|---|---|
| | | pound A | (10b) |
| | | Plasma | Plasma |
| | Day 21 | concen- | concen- |
| | TGI | tration | tration |
| Treatments | (%) | (μM) | (μM) |
| Vehicle | 0% | BQL | BQL |
| Compound A 100 mg/kg QD | 137% | 8.77 ± 7.25 | BQL |
| Compound A 30 mg/kg QD | 126% | 2.02 ± 0.31 | BQL |
| Formula (10b) 100 mg/kg QD | 89% | BQL | 7.54 ± 1.60 |
| Formula (10b) 50 mg/kg QD | 46% | BQL | 4.44 ± 1.05 |
| Compound A 100 mg/kg QD + | 136% | 7.79 ± 3.40 | 5.41 ± 3.43 |
| Formula (10b) 100 mg/kg QD | | | |
| Compound A 100 mg/kg QD + | 131% | 9.20 ± 5.29 | 5.52 ± 1.46 |
| Formula (10b) 50 mg/kg QD | | | |
| Compound A 30 mg/kg QD + | 133% | 2.70 ± 0.92 | 8.27 ± 1.18 |
| Formula (10b) 100 mg/kg QD | | | |

TGI % = (Ci-Ti)/(Ci-C₀) *100%. Ti and Ci are the mean tumor volumes of the treatment and vehicle groups on the measurement day, respectively; C₀ denotes the mean tumor volume of the vehicle group on Day 0.
For plasma concentrations, data represent mean ± SD.
N = 4 mice/group.
BQL, below quantitation limit (3 nM).

Pharmacokinetic analysis (Table 2) was conducted 2 hours after the final dose of Compound A, which was 8 hours after the final dose of formula (10b). Significant 10 concentrations of Compound A in the plasma were detected, with 30 mg/kg giving rise to 2.0 μM, and 100 mg/kg giving rise to 8.8 μM at 2 hours after the final dose. Compound A plasma concentration was not impacted by treatment with formula (10b). Formula (10b) 100 mg/kg gave rise to similar significant plasma concentration when administered as a single agent (7.5 μM) and in combination with Compound A (5-10 μM), 8 hours after dosing. Formula (10b) 50 mg/kg dosing also resulted in similar significant plasma concentration when administered as a single agent (4.4 μM) and in combination with Compound A (5.5 μM), 8 hours after dosing. The data suggest no drug-drug interactions between Compound A and formula (10b) in mice.

TABLE 3
Summary of mouse morbidity in the efficacy
study presented in FIGS. 1B-1E.

| | Mouse loss and observations |
|---|---|
| Treatment Groups | (n = 8 mice/group) |
| Vehicle | None |
| Compound A 100 mg/kg QD | None |
| Compound A 30 mg/kg QD | None |
| Formula (10b) 100 mg/kg QD | Day 18 (Found dead) |
| | Day 21 (Found dead) |
| Formula (10b) 50 mg/kg QD | Day 3 (BWL) |
| | Day 12 (BWL) |
| | Day 19 (BWL) |
| Compound A 100 mg/kg QD + | Day 8 (Found dead) |
| Formula (10b) 100 mg/kg QD | Day 15 (Found dead) |
| | Day 16 (Found dead) |
| Compound A 100 mg/kg QD + | Day 15 (BWL) |
| Formula (10b) 50 mg/kg QD | Day 17 (Found dead) |
| Compound A 30 mg/kg QD + | Day 10 (BWL) |
| Formula (10b) 100 mg/kg QD | |

Each event (mouse loss) indicates loss of one mouse, with the day indicating the dosing day on which the mouse was removed from the study.
BWL, body weight loss;
QD, dosed once every day.

### D. Conclusions

In the NCI-H358 NSCLC xenograft model, which harbors a KRAS G12C mutation, treatment with formula (10b) in combination with Compound A robustly inhibited tumor growth. In detail, treatment with Compound A 10 mg/kg QD as a single agent resulted in tumor growth inhibition, and combination with formula (10b) resulted in tumor regression. Treatment with higher doses of Compound A (30 mg/kg QD or 100 mg/kg QD) as a single agent caused robust tumor regression; combination with formula (10b) did not notably further suppress tumor volume. Treatment with formula (10b) monotherapy at 100 mg/kg QD causes approximately tumor stasis. All treatment conditions tested are well tolerated in the mouse model.

### Example 2: In Vivo Efficacy of the Combination of Formula (10b) and Sotorasib

### A. Materials

### Test Article #1—Formula (L1b):

Chemical name: 6-[(3S,4S)-4-amino-3-methyl-2-oxa-8-azaspiro [4.5]decan-8-yl]-3-(2,3-dichlorophenyl)-2,5-dimethyl-3,4-dihydropyrimidin-4-one
Molecular formula: C₂₁H₂₆Cl₂N₄O₂
Molecular weight: 437.37

### Test Article #2—Sotorasib:

CAS #: 2296729-00-3
Molecular formula: C₃₀H₃₀F₂N₆O₃
Molecular weight: 560.6

### B. Experimental Procedures

Tumor Cell Line Culture. The human NSCLC NCI-H358 cell line (Shanghai Institutes for Biological Sciences of Chinese Academy of Sciences) was maintained in vitro with RPMI-1640 medium (Gibco, catalog C22400500BT) supplemented with 10% heat inactivated fetal bovine serum (Gibco, catalog 10099-141C) in a 37° C. chamber with 5% CO₂. Passage 9 NCI-H358 cells were thawed and expanded, and passage 13 cells were harvested for inoculations. The human NSCLC NCI-H2122 cell line (American Type Culture Collection) was maintained in vitro with RPMI-1640 medium (Gibco, catalog C22400500BT) supplemented with 10% heat inactivated fetal bovine serum (Gibco, catalog 10091-148) in a 37° C. chamber with 5% CO₂. Passage 6 NCI-H2122 cells were thawed and expanded, and passage 14 cells were harvested for inoculations.

Animal Inoculations and Randomization. All experiments were reviewed and approved by the Crown Bioscience, Inc., Institutional Animal Care and Use Committee (IACUC) prior to execution and performed in accordance with the regulations of the Association for Assessment and Accreditation of Laboratory Animal Care (AAALAC). For the NCI-H358 study, 75 NOD/SCID female mice (Beijing Anikeeper Biotech Co., Ltd) at 6 to 8 weeks of age were inoculated subcutaneously in the right front flank region with NCI-H358 tumor cells (5×10⁶ cells per mouse), suspended in a 1:1 ratio of PBS to Matrigel (Corning, catalog 354234) in a volume of 0.1 mL per mouse. Mice were randomized into four groups (10 mice/group) by tumor volume (size mm³) 13 days post cell inoculation when the mean tumor volume was 160 mm³. For the NCI-H2122 study, 80 BALB/c nude female mice (GemPharmatech Co., Ltd) at 6 to 8 weeks of age were inoculated subcutaneously in the right front flank region with NCI-H2122 tumor cells (1×10⁷ cells per mouse), suspended in a 1:1 ratio of PBS to Matrigel (Corning, catalog 356234) in a volume of 0.1 mL per mouse. Mice were randomized into four groups (10 mice/group) by tumor volume (size mm³) 9 days post cell inoculation when the mean tumor volume was 180 mm³.

Dosing. Four groups of NCI-H358 tumor-bearing female NOD/SCID mice (10 animals/group) were administered one of the following treatment regimens 1) vehicle, 2) formula (10b) (100 mg/kg), 3) sotorasib (10 mg/kg), or 4) a combination of formula (10b) (100 mg/kg) and sotorasib (10 mg/kg) once daily by oral gavage for a period of 28 days. Four groups of NCI-H2122 tumor-bearing female BALB/c nude mice (10 animals/group) were administered one of the following treatment regimens 1) vehicle, 2) formula (10b) (100 mg/kg), 3) sotorasib (100 mg/kg), or 4) a combination of formula (10b) (100 mg/kg) and sotorasib (100 mg/kg) once daily by oral gavage for a period of 21 days.

Formula (10b) Formulation. Formula (10b) formulation buffer (0.5% v/v methyl cellulose in sterile deionized water) was prepared by weighing the desired amount of methyl cellulose (Sigma-Aldrich, catalog M0262, viscosity 400 cP) into a glass bottle. Sterile deionized water equivalent to 75% v/v of the intended final volume was added under continuous magnetic stirring with a stir bar and stirred at room temperature until complete dissolution. The buffer was then brought to the final volume with sterile deionized water. A correction factor of 1.025 was applied to the formula (10b) formulation to accommodate for the purity (97.7%).

Formula (10b) working suspensions of 10 mg/mL (10.25 mg/mL with correction factor) were prepared and administered at a 10 mL/kg dose volume by oral gavage to mice for the 100 mg/kg doses. To prepare formula (10b) formulations, compound was accurately weighed into a glass vial. Formula (10b) formulation buffer equivalent to 70% v/v of the intended final volume was added to the glass vial containing drug substance and mixed well using a ¼-inch probe for 4 to 9 minutes until a homogeneous suspension was achieved with no large visible agglomerates/particles. The rest of the suspending vehicle was added to reach the intended final volume to the dispersion containing drug substance. The suspension was mixed well for 30 minutes, stored at 4° C. for one week, and well mixed prior to and throughout dosing each day.

Sotorasib Formulation. Sotorasib formulation buffer (2% v/v HPMC/1% v/v Tween 80 in sterile deionized water) was prepared by weighing the desired amount of HPMC (Sigma-Aldrich, catalog H3785) and Tween 80 (Sigma-Aldrich, catalog P4780) into a glass bottle. Sterile deionized water equivalent to 80% v/v of the intended final volume was added under continuous magnetic stirring with a stir bar and stirred at room temperature until complete dissolution. The buffer was then brought to the final volume with sterile deionized water.

Sotorasib working suspensions of 1 mg/mL and 10 mg/mL were prepared and administered at a 10 mL/kg dose volume by oral gavage to mice for the 10 mg/kg and 100 mg/kg doses, respectively. To prepare sotorasib formulations, compound was accurately weighed into a glass vial. Sotorasib formulation buffer equivalent to 100% v/v of the intended final volume was added to the glass vial containing drug substance and mixed well until a homogeneous suspension was achieved with no large visible agglomerates/particles. The suspension was mixed well and the working suspension was dosed as soon as possible. The suspension was prepared daily and well mixed prior to and throughout dosing.

Tumor and Body Weight Measurements. Tumor volumes were measured two times per week after randomization in two dimensions using a caliper, and the volume was expressed in mm³ using the formula: Tumor volume=(length×width×width)/2, where length was defined as the longest tumor dimension and width was defined as the longest tumor dimension perpendicular to the length. Dosing as well as tumor and body weight measurements were conducted in a Laminar flow cabinet. Individual mouse tumor volumes (and mean tumor volumes (±standard error of the mean (SEM)) for each group were recorded. Tumor growth inhibition (TGI), an indicator of anti-tumor effectiveness, was calculated for each treatment (T) versus control (C) group using the day 1 (0) and indicated measurement day (i) mean tumor volume measurements with the formula: TGI (%)=(1−(Ti−T0)/(Ci−C0))×100. TGI was calculated on the day after the last continuous daily dose (day 29) and only reported if ≤100%. Tumor regression (REG) was also assessed and defined as a tumor with a smaller tumor volume on the indicated day of the study compared to the first day of dosing on day 1. If the TGI was >100%, mean tumor regression was reported instead and calculated by determining the mean percentage of tumor regression on the day after the last continuous daily dose as indicated compared to the first dose on day 1.

Body weights (BW) were also recorded twice weekly. Individual mouse body weights and body weight change, expressed in percentage, were calculated for each animal using the day 1 (0) body weight on the first day of dosing and the body weight on the indicated (i) day after dosing using the following formula: BW change (%)=((BWi/BW0)×100)−100. Mean body weight changes (±SEM) were recorded for group. Treatments were not considered tolerated if >20% of the mice in the group had ≥20% body weight loss or >20% of the mice in group spontaneously died or had any clinical signs of distress that required euthanasia.

Animal tumor and body weight measurement data were recorded and stored using Study Director™ software (version 3.1.399.19). All analyses were performed using GraphPad Prism software (version 9) or calculated from the raw data in Microsoft Excel.

Statistical analyses. Statistical analyses were performed with GraphPad Prism software (version 9). For statistical analyses comparing the vehicle group to all other groups, two-way repeated-measures analysis of variance (ANOVA) followed by post hoc Tukey's multiple comparisons test of the means was applied over the indicated number of days. For statistical analyses comparing the monotherapy and combination groups, two-way repeated-measures ANOVA was performed between each monotherapy group mean and combination group mean over the number of indicated days. A p value of less than 0.05 was considered statistically significant.

Experimental Endpoints. Animal health was monitored at least twice weekly through behavioral observations and weight checks. After tumor inoculation, the animals were checked daily for morbidity and mortality. During routine monitoring, the animals were checked for any effects of tumor growth and treatments on behavior such as mobility, food and water consumption, body weight gain/loss, eye/hair matting, and any other abnormalities.

Study endpoint was defined when animals reached one of the following criteria for euthanasia: ≥20% body weight loss at any time versus day 1, ≥15% body weight loss for 72 hours versus day 1, individual tumor volume of ≥3000 mm³, or all mice in each group were euthanized when the mean tumor volume reached ≥2000 mm³. In the NCI-H358 study, animals in all groups were euthanized on day 34 or day 35 and in the NCI-H2122 study, animals in all groups were euthanized on day 21 or day 22.

### C. Results

Sotorasib (AMG 510) is a KRAS covalent inhibitor that specifically targets the KRAS G12C mutant. It has demonstrated single agent response in patients with solid tumors, including NSCLC, harboring KRAS G12C mutation. Specifically, in the CodeBreaK 100 study, the subgroup with NSCLC had a confirmed objective response rate (complete or partial response) of 32.2% and a disease control rate (objective response or stable disease) of 88.1%; the median progression-free survival was 6.3 months. Hong et al., N. Engl. J. Med. 2020; 383:1207-1217. Cell line studies have shown that SHP2 inhibitors increased KRAS-GDP occupancy and enhanced KRAS G12C inhibitor efficacy as the current generation of KRAS G12C inhibitors only inhibit the GDP-bound form of KRAS G12C. Unbiased functional genomics approaches have identified SHP2 as a vulnerability under KRAS G12C inhibition. The addition of SHP2 inhibition to therapeutics that target mutant KRAS G12C may improve efficacy in several ways. Because mutant KRAS G12C retains some level of cycling between these two states, the GTP-bound form of KRAS G12C can still activate ERK signaling in the presence of sotorasib. SHP2 inhibition decreases SOS1-dependent GTP loading of RAS, which leads to elevated RAS-GDP levels and may therefore enhance the efficacy of sotorasib. In addition, compensatory bypass signaling feedback activation of either upstream or downstream mediators of the RTK/MAPK pathway has been demonstrated to reduce effectiveness of KRAS G12C inhibitors both preclinically and clinically, and SHP2 inhibition may inhibit this feedback activation.

SHP2 inhibition may also enhance anti-tumor activity of KRAS G12C inhibitors in patients with molecular subtypes where outcomes have been particularly poor. For example, mutations in the tumor suppressor Kelch-like ECH-associated protein 1 (KEAP1) are found in approximately 20% of KRAS mutant NSCLC, and co-mutation of KRAS and KEAP1 is associated with poorer prognosis and clinical outcomes.

Two studies were performed to determine if the combination of the SHP2 inhibitor formula (10b) and sotorasib enhanced anti-tumor activity over either agent alone. Studies were performed with the NSCLC NCI-H358 cell line-derived xenograft model, which bears a KRAS G12C mutation and the NSCLC NCI-H2122 cell line-derived xenograft model, which bears KRAS G12C, KEAP1, and STK11 co-mutations.

Treatment with the combination of formula (10b) and sotorasib resulted in tumor regressions in the NSCLC NCI-H358 subcutaneous xenograft model harboring a KRAS G12C mutation. An in vivo study was conducted to evaluate the impact of formula (10b) in combination with the KRAS G12C inhibitor, sotorasib, on tumor volume in female NOD/SCID mice bearing the cell line-derived NCI-H358 NSCLC xenograft model, which harbors a KRAS G12C mutation. Female NOD/SCID mice were implanted subcutaneously with NSCLC NCI-H358 tumor cells. When tumors reached a mean size of 160 mm³, mice were randomized into treatment groups (n=10 per group) and dosed by oral gavage once each day with the indicated levels of vehicle, 100 mg/kg formula (10b), 10 mg/kg sotorasib, or the combination of 100 mg/kg formula (10b) and 100 mg/kg sotorasib from day 1 to day 28. Mouse tumor volumes were measured twice weekly until day 34 and results are shown through day 29.

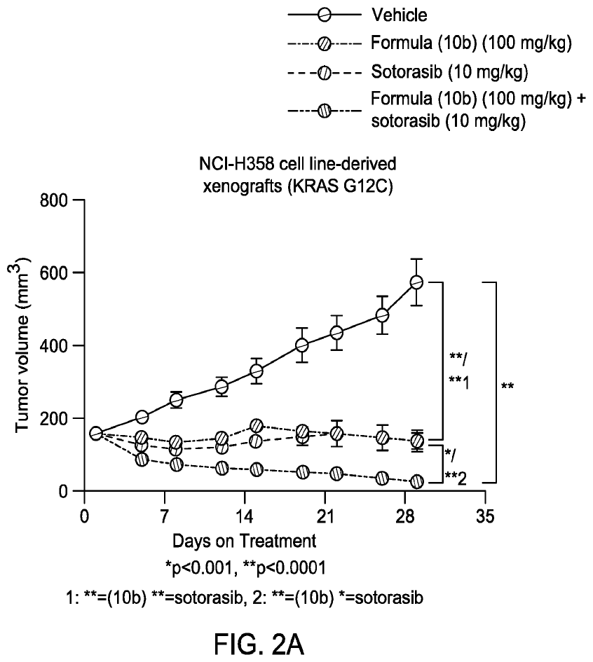

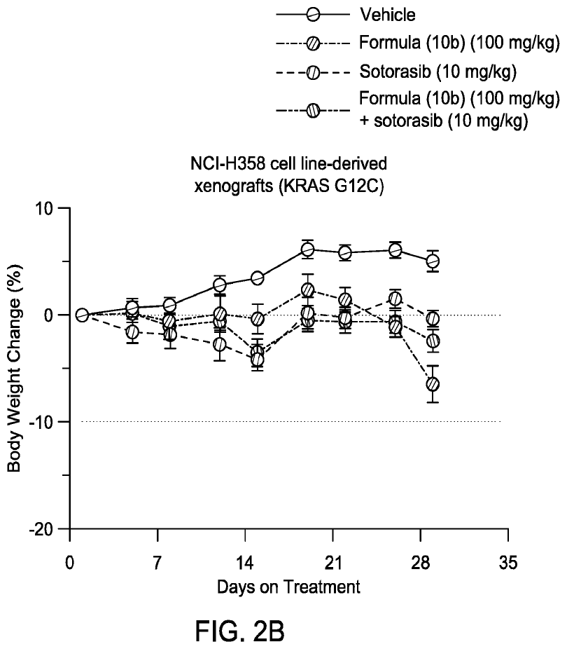

As shown in FIG. 2A and Table 4, the combination of formula (10b) and sotorasib showed significantly greater anti-tumor activity than treatment with either test article as monotherapy. Following once daily oral administration of formula (10b) (100 mg/kg) and sotorasib (10 mg/kg) in combination, a statistically significant tumor volume reduction was observed in the combination group compared to both monotherapy groups (FIG. 2A and Table 4) and the day 29 mean tumor regression was significantly increased to 83% (Table 4). The results also showed that both formula (10b) and sotorasib had monotherapy anti-tumor activity at the tested dose levels. Following once daily oral administration of formula (10b) (100 mg/kg) or sotorasib (10 mg/kg), a statistically significant reduction in tumor volume compared to the vehicle group was observed for both treatment groups (FIG. 2A and Table 4), with day 29 mean tumor regressions of 14% and 13% for the formula (10b) and sotorasib monotherapy groups, respectively.

TABLE 4
Summary of Anti-tumor Activity of Formula (10b) and Sotorasib
as Monotherapy and in Combination in NOD/SCID Mice Bearing
NCI-H358 Subcutaneous Cell Line-Derived Tumors

| | | | Day 29 | | | | |
|---|---|---|---|---|---|---|---|
| | | Dose | Mean Tumor | Mean | p value | p value vs | |
| Group | | Level | Volume (mm³) | Tumor | vs | Formula | p value vs |
| (n = 10) | Treatment | (mg/kg) | (±SEM) | REG | Vehicle* | (10b)** | Sotorasib** |
| 1 | Vehicle | — | 574 (±64) | — | — | — | — |
| 2 | Formula | 100 | 138 (±17) | 14% | <0.0001 | — | — |
| | (10b) | | | | | | |
| 3 | Sotorasib | 10 | 139 (±27) | 13% | <0.0001 | — | — |
| 4 | Formula | 100 | 27 (±3) | 83% | <0.0001 | <0.0001 | 0.0004 |
| | (10b) | | | | | | |
| | Sotorasib | 10 | | | | | |

Abbreviations: ANOVA = analysis of variance; NOD/SCID = non-obese diabetic/severe combined immunodeficiency; n = number; REG = regression; SEM = standard error of the mean.
Note:
Daily oral dosing was performed from day 1 to day 28 and the last tumor measurement was performed on day 34. For the statistical analyses, 2-way repeated-measures ANOVA of group means from day 5 to 29 followed by post hoc Tukey's multiple comparisons test was performed for the vehicle group comparisons* and 2-way repeated-measures ANOVA of the monotherapy and combination group means from day 5 to 29 were performed for the combination group comparisons**.

As shown in FIG. 2B, there was no impact of the treatments on body weights and all treatments were well tolerated in the mouse model.

Treatment with the combination of formula (10b) and sotorasib results in tumor growth inhibition in the NSCLC NCI-H2122 subcutaneous xenograft model harboring KRAS G12C, KEAP1, and STK11 co-mutations. An in vivo study was conducted to evaluate the impact of formula (10b) in combination with the KRAS G12C inhibitor, sotorasib, on tumor volume in BALB/c nude mice bearing the cell line-derived NCI-H2122 NSCLC xenograft model, which harbors a KRAS G12C mutation. The model also has several co-occurring mutations including a frameshift insertion in KEAP1 (KEAP1 A203fs), and a frameshift deletion in STK11 (STK11 G279fs), which cause loss of function of KEAP1 and STK11. When tumors reached a mean size of 180 mm³, mice were randomized into treatment groups (n=10 per group) and dosed by oral gavage once each day with the indicated levels of vehicle, 100 mg/kg formula (10b), 100 mg/kg sotorasib, or the combination of 100 mg/kg formula (10b) and 100 mg/kg sotorasib from day 1 to day 21. Mouse tumor volumes and body weights were measured twice weekly until day 21.

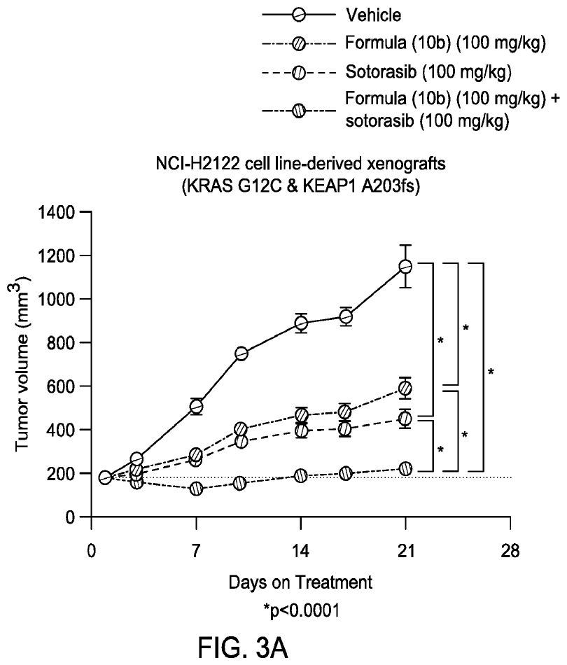

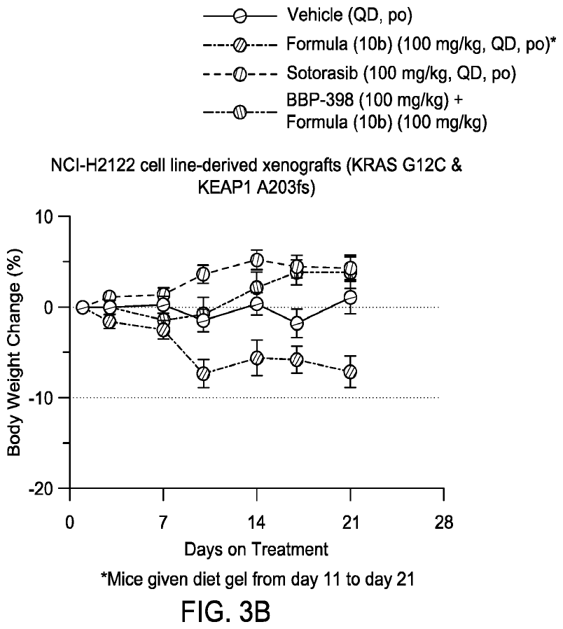

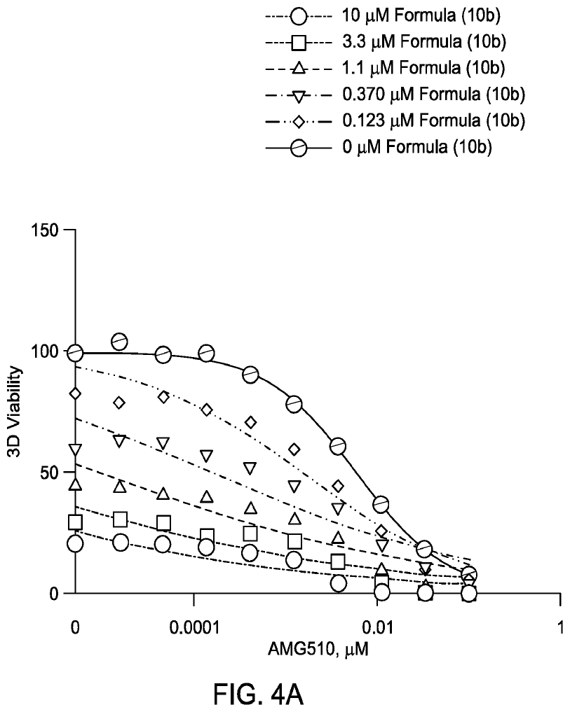

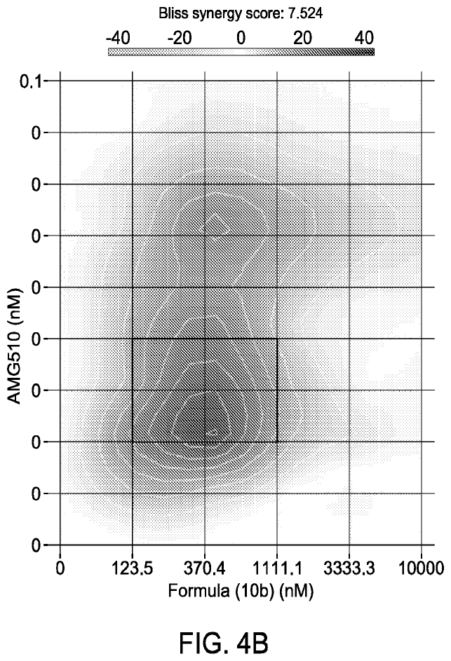

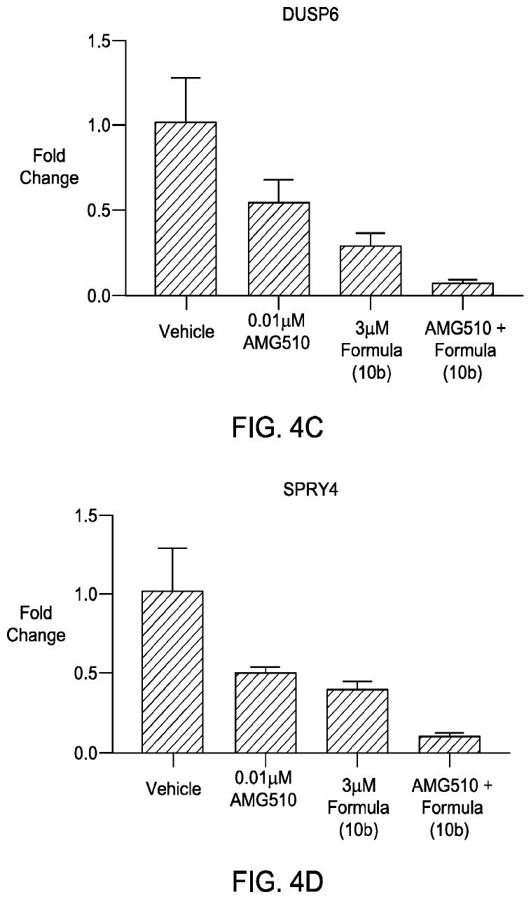

As shown in FIG. 3A and Table 5, the combination of formula (10b) and sotorasib showed significantly greater anti-tumor activity than treatment with either test article as monotherapy. Following once daily oral administration of formula (10b) (100 mg/kg) and sotorasib (100 mg/kg) in combination, a statistically significant tumor volume reduction was observed in the combination group compared to both monotherapy groups (FIG. 3A and Table 5) and the day 21 tumor growth inhibition was significantly increased to 96% (Table 5). The results also showed that both formula (10b) and sotorasib had monotherapy anti-tumor activity at the tested dose levels. Following once daily oral administration of formula (10b) (100 mg/kg) or sotorasib (100 mg/kg), a statistically significant reduction in tumor volume compared to the vehicle group was observed for both treatment groups (FIG. 3A and Table 5), with day 21 tumor growth inhibition of 58% and 72% for the formula (10b) and sotorasib monotherapy groups, respectively (Table 5).

TABLE 5
Summary of Anti-tumor Activity of Formula (10b) and Sotorasib
as Monotherapy and in Combination in BALB/c Nude Mice Bearing
NCI-H2122 Subcutaneous Cell Line-Derived Tumors

| | | | Day 21 | | | | |
|---|---|---|---|---|---|---|---|
| | | Dose | Mean Tumor | | p value | p value vs | |
| Group | | Level | Volume (mm³) | | vs | formula | p value vs |
| (n = 10) | Treatment | (mg/kg) | (±SEM) | TGI | Vehicle* | (10b)** | Sotorasib** |
| 1 | Vehicle | — | 1149 (±98) | — | — | — | — |
| 2 | Formula | 100 | 590 (±48) | 58% | p < 0.0001 | — | — |
| | (10b) | | | | | | |
| 3 | Sotorasib | 100 | 449 (±47) | 72% | p < 0.0001 | — | — |
| 4 | Formula | 100 | 221 (±12) | 96% | p < 0.0001 | p < 0.0001 | p < 0.0001 |
| | (10b) | | | | | | |
| | Sotorasib | 100 | | | | | |

Abbreviations: ANOVA = analysis of variance; BALB = Bagg Albino; n = number; SEM = standard error of the mean; TGI = tumor growth inhibition.
Note:
Daily oral dosing was performed from day 1 to day 21 and the last tumor measurement was performed on day 21. For the statistical analyses, 2-way repeated-measures ANOVA of group means from day 3 to 21 followed by post hoc Tukey's multiple comparisons test was performed for the vehicle group comparisons* and 2-way repeated-measures ANOVA of the monotherapy and combination group means from day 3 to 21 were performed for the combination group comparisons**.

As shown in FIG. 3B, there was no impact of the sotorasib monotherapy or combination treatments on body weights and these treatments were well tolerated in the mouse model. Unexpectedly, several mice in the formula (10b) monotherapy group had body weight loss over 10%, therefore, the animals in this group were placed on diet gel from day 11 to day 21 (FIG. 3B). The cause of this weight loss is unknown and has not been observed in previous studies at this dose level (100 mg/kg). Importantly, formula (10b) was administered at the same dose level (100 mg/kg) in the combination group of formula (10b) and sotorasib, and the combination was well tolerated (FIG. 3B).

### D. Conclusions

The results from these two in vivo studies demonstrate that formula (10b) in combination with sotorasib was well tolerated and showed a significant anti-tumor benefit in the mutant KRAS G12C NSCLC NCI-H358 cell line-derived xenograft model and the mutant KRAS G12C and KEAP1 A203fs NSCLC NCI-H2122 cell line-derived xenograft model. These in vivo studies support the clinical evaluation of the combination and show that SHP2 inhibition may potentiate the response of KRAS G12C inhibitors and increase the number of patients, with mutant KRAS G12C with or without co-mutations such as KEAP1 or STK11, that may benefit from formula (10b) as a potential treatment in combination with sotorasib.

### Example 3: A Phase 1A/1B Study of the SHP2 Inhibitor Formula (10b) in Combination with the KRAS G12C Inhibitor Sotorasib in Patients with Solid Tumors with a KRAS G12C Mutation

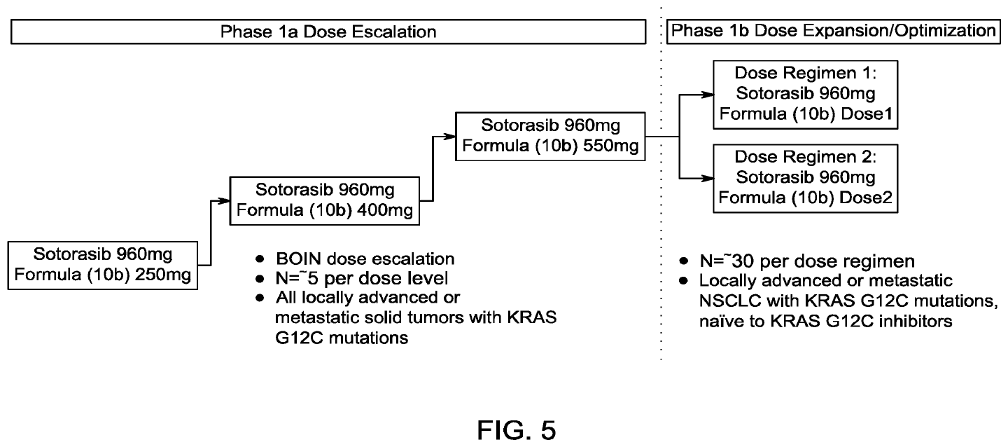

FIG. 5 shows a Phase 1A/1B study of formula (10b) in combination with sotorasib in patients with solid tumors with a KRAS G12C mutation. The study design includes Phase 1a dose escalation and Phase 1b dose expansion/optimization.

TABLE 6
Schedule of Assessments: Phase 1a Dose Escalation and Phase 1b Dose Expansion/Optimization

| | | | | | | | | | | Subse- |
|---|---|---|---|---|---|---|---|---|---|---|
| | | | | | | | | | | quent |
| | Pre- | | | | | | | | | ODD |
| | Screen- | Screen- | | | | | | | | Cycles (eg, |
| | ing | ing | Cycle 1 | | | | | Cycle 2^{BB} | | 3, 5)^{BB} |
| Study Day ^{A} | | −30 | 1 ^{U, V} | 2 | 8 | 15 | 22^{JJ} | 1 ^{U} | 15 | 1 ^{U} |
| | | to −1 | | | | | | | | |
| +/− days allowable ^{A} | | 0 | 0 | 0 | ±3 | ±3 | −1 | ±3 | ±3 | ±3 |
| | | | | | | | to +3 | | | |
| Molecular | | | | | | | | | | |
| Prescreening ^{KK} | | | | | | | | | | |
| Informed consent | X | | | | | | | | | |
| Testing | X | | | | | | | | | |
| Study Consent | | | | | | | | | | |
| Informed consent | | X | | | | | | | | |
| Confirmation of | | | | | | | | | | |
| Disease State ^{D} | | | | | | | | | | |
| Locally advanced or | | X | | | | | | | | |
| metastatic solid | | | | | | | | | | |
| tumors (Phase 1a) | | | | | | | | | | |
| Advanced NSCLC | | X | | | | | | | | |
| (Phase 1b) | | | | | | | | | | |
| Confirmation of | | | | | | | | | | |
| Tumor Mutation ^{C} | | | | | | | | | | |
| Documented KRAS- | | X | | | | | | | | |
| G12C mutation | | | | | | | | | | |
| Administration of | | | | | | | | | | |
| Study Treatment ^{B} | | | | | | | | | | |
| Compound (10b) | | | X-----------------------------------------------------------------------------X | | | | | | | |
| Sotorasib | | | X-----------------------------------------------------------------------------X | | | | | | | |
| Reconciliation | | | | | X | X | X | X | X | X |
| between patient | | | | | | | | | | |
| diary and pill | | | | | | | | | | |
| count | | | | | | | | | | |
| DLT | | | | | | | | | | |
| Assessment Period | | | | | | | | | | |
| (Phase 1a Only) | | | | | | | | | | |
| DLT Assessment | | | X---------------------------X | | | | | | | |
| Period ^{NN} | | | | | | | | | | |
| Disease Assessment | | | | | | | | | | |
| Restaging scan (CT, | | X | | | | | | | | X |
| MRI, or PET-CT) ^{E} | | | | | | | | | | (±7 d) |
| Relevant tumor | | X | | | | | | | | X |
| marker ^{F} | | | | | | | | | | |
| Study Procedures | | | | | | | | | | |
| & Examinations | | | | | | | | | | |
| Eligibility ^{G} | | X ^{G} | | | | | | | | |
| Demographics ^{H} | | X | | | | | | | | |
| Medical history ^{I} | | X | X^{S} | | | | | | | |
| ECG | | X ^{FF} | X^{W, FF} | X ^{Z} | | | | X^{W, FF} | | X^{W, FF} |
| MUGA/ECHO | | X | | | | | | X ^{CC} | | X ^{CC} |
| Physical | | X | X^{S} | | X | | | X | | X |
| Examination ^{J} | | | | | | | | | | |
| ECOG performance | | X | X ^{X, Y} | | | | | X | | X |
| status | | | | | | | | | | |
| Vital signs ^{K} | | X | X ^{Y} | X ^{Z} | X ^{Y} | X ^{Y} | | X ^{Y} | X ^{Y} | X ^{Y} |
| Prior/Concomitant | | Document any medication or vaccine that the patient is receiving at the time of signing the | | | | | | | | |
| medications ^{LL} | | ICF and/or within 30 d or 5 half-lives (whichever is longer) of first dose of study treatment. | | | | | | | | |
| | | Document concomitant medications taken from time of signing the ICF through 28 d after the | | | | | | | | |
| | | last dose of study treatment or date of the decision to permanently discontinue study treatment. | | | | | | | | |
| AE reporting ^{L} | SAEs | SAEs | Document AEs and SAEs from first dose of study treatment through at least 28 d after | | | | | | | |
| | only | only | last dose of treatment. AE information will be collected at study visits or over the phone. | | | | | | | |
| Survival | | | | | | | | | | |
| Anticancer therapies | | | | | | | | | | |
| since drug | | | | | | | | | | |
| discontinuation | | | | | | | | | | |
| Laboratory | | | | | | | | | | |
| Evaluations ^{M} | | | | | | | | | | |
| FSH ^{N} | | X | | | | | | | | |
| Pregnancy test ^{O} | | X | X | | | | | X | | X |
| HIV, HBV & HCV | | X | | | | | | | | |
| Blood hematology ^{P} | | X | X ^{Y} | X ^{Z} | X ^{Y} | X ^{Y} | | X ^{Y} | X ^{Y} | X ^{Y} |
| Chemistry ^{Q} | | X | X ^{Y} | X ^{Z} | X ^{Y} | X ^{Y} | | X ^{Y} | X ^{Y} | X ^{Y} |
| Coagulation | | X | X^{W} | X ^{Z} | | | | X^{W} | | X ^{Y} |
| (aPTT, PT, INR) | | | | | | | | | | |
| Urinalysis (spot | | X | X ^{Y} | | | | | X ^{Y} | | X ^{Y} |
| collection) | | | | | | | | | | |
| Research Studies (PK/ | | | | | | | | | | |
| Pharmacodynamic) ^{MM} | | | | | | | | | | |
| Comp. (10b) | | | X ^{AA} | X ^{Z} | X ^{Y} | X ^{Y} | | X ^{AA} | | X ^{EE} |
| samples | | | | | | | | | | |
| Sotorasib PK | | | X ^{AA} | X ^{Z} | X ^{Y} | X ^{Y} | | X ^{AA} | | X ^{EE} |
| samples | | | | | | | | | | |
| Circulating tumor | | X | | | | | | X | | X |
| markers ^{R} | | | | | | | | | | |
| Fresh tumor biopsy | | X^{T} | | | | | | X ^{DD} | | |

| | Subse- | | | |
|---|---|---|---|---|
| | quent | | | |
| | EVEN | | | Long- |
| | Cycles (eg, | | EOT | Term |
| | 4, 6)^{BB} | ET | Follow-up | Follow-up |
| Study Day ^{A} | 1 ^{U} | NA | Last | Every 3 |
| | | | Dose +28 d | months |
| +/− days allowable ^{A} | ±3 | NA | ±3 | NA |
| Molecular | | | | |
| Prescreening ^{KK} | | | | |
| Informed consent | | | | |
| Testing | | | | |
| Study Consent | | | | |
| Informed consent | | | | |
| Confirmation of | | | | |
| Disease State ^{D} | | | | |
| Locally advanced or | | | | |
| metastatic solid | | | | |
| tumors (Phase 1a) | | | | |
| Advanced NSCLC | | | | |
| (Phase 1b) | | | | |
| Confirmation of | | | | |
| Tumor Mutation ^{C} | | | | |
| Documented KRAS- | | | | |
| G12C mutation | | | | |
| Administration of | | | | |
| Study Treatment ^{B} | | | | |
| Compound (10b) | X-----------------------------------------------------------------------------X | | | |
| Sotorasib | X-----------------------------------------------------------------------------X | | | |
| Reconciliation | X | | X | |
| between patient | | | | |
| diary and pill | | | | |
| count | | | | |
| DLT | | | | |
| Assessment Period | | | | |
| (Phase 1a Only) | | | | |
| DLT Assessment | | | | |
| Period ^{NN} | | | | |
| Disease Assessment | | | | |
| Restaging scan (CT, | | | X (±7 | |
| MRI, or PET-CT) ^{E} | | | d) ^{GG} | |
| Relevant tumor | | | X | |
| marker ^{F} | | | | |
| Study Procedures | | | | |
| & Examinations | | | | |
| Eligibility ^{G} | | | | |
| Demographics ^{H} | | | | |
| Medical history ^{I} | | | | |
| ECG | | X | X | |
| MUGA/ECHO | | X ^{CC, HH} | X ^{CC, HH} | |
| Physical | X | X | X | |
| Examination ^{J} | | | | |
| ECOG performance | X | X | X | |
| status | | | | |
| Vital signs ^{K} | X ^{Y} | X | X | |
| Prior/Concomitant | Document any medication or vaccine that the patient is receiving at the time of signing the | | | |
| medications ^{LL} | ICF and/or within 30 d or 5 half-lives (whichever is longer) of first dose of study treatment. | | | |
| | Document concomitant medications taken from time of signing the ICF through 28 d after the | | | |
| | last dose of study treatment or date of the decision to permanently discontinue study treatment. | | | |
| AE reporting ^{L} | Document AEs and SAEs from first dose of study treatment through at least 28 d after | | | |
| | last dose of treatment. AE information will be collected at study visits or over the phone. | | | |
| Survival | | | | X ^{II} |
| Anticancer therapies | | | | X ^{II} |
| since drug | | | | |
| discontinuation | | | | |
| Laboratory | | | | |
| Evaluations ^{M} | | | | |
| FSH ^{N} | | | | |
| Pregnancy test ^{O} | X | X | X | |
| HIV, HBV & HCV | | | | |
| Blood hematology ^{P} | X ^{Y} | X | X | |
| Chemistry ^{Q} | X ^{Y} | X | X | |
| Coagulation | X ^{Y} | X | X | |
| (aPTT, PT, INR) | | | | |
| Urinalysis (spot | | X | X | |
| collection) | | | | |
| Research Studies (PK/ | | | | |
| Pharmacodynamic) ^{MM} | | | | |
| Comp. (10b) | X ^{EE} | | | |
| samples | | | | |
| Sotorasib PK | X ^{EE} | | | |
| samples | | | | |
| Circulating tumor | X | | X | |
| markers ^{R} | | | | |
| Fresh tumor biopsy | | | X (±7 d) | |

### Abbreviations and Footnotes of Table 6

Abbreviations: AE=adverse event; aPTT=activated partial thromboplastin time; ctDNA=circulating tumor deoxyribonucleic acid; CR=complete response(s); CT=computed tomography; ECG=electrocardiogram; ECHO=echocardiogram; ECOG=Eastern Cooperative Oncology Group; EOS=End of study; EOT=End of treatment; ET=Early Termination; DLT=dose-limiting toxicity; FDA=Food and Drug Administration; FSH=follicle-stimulating hormone; FU=Follow-Up; HBV=hepatitis B virus; HCV=hepatitis C virus; HIV=human immunodeficiency virus; ICF=informed consent form; INR=international normalized ratio; IV=intravenous; KRAS=Kirsten rat sarcoma viral oncogene homolog; LVEF=left ventricular ejection fraction; MRI=magnetic resonance imaging; MUGA=multiple-gated acquisition scan; NA=not applicable; NSCLC=non-small cell lung cancer; PD=progressive disease; PET=positron emission tomography; PK=pharmacokinetic; PR=partial response; PT=prothrombin time; QD=once daily; QTcF=QT using Fridericia's correction formula; RECIST=Response Evaluation Criteria in Solid Tumors; RP1bD=recommended Phase 1b dose
A. Due to the Coronavirus Disease 2019 (COVID-19) pandemic, telephone or video contact visits for safety monitoring, alteration of the visit schedule, or other appropriate clinical trial conduct changes, may occur. The study days allowable are intended as a best practice; however, may be adjusted as required. If necessary, additional changes to the schedule of events will be performed and documented consistent with current FDA guidance (e.g., FDA Guidance of Clinical Trials of Medical Products during COVID-19 Public Health Emergency).
B. In each 28-day cycle, study treatments will be taken orally every 24 hours (±3 hours). Compound (10b) should be taken before sotorasib. The day before and the day of study visit, both study treatments should be taken within a 5-minute window unless one of the study treatments is on hold. Study treatments should be taken with approximately 240 mL (8 oz) of water after an overnight fast (minimum 8 hr) followed by 2 hr of fasting after the dose is taken. Water is allowed ad lib except 1 hour before and after dosing. Compound (10b) will be taken QD, in combination with sotorasib QD. Two doses of Compound (10b) selected from Phase 1a Dose Escalation will be used for Compound (10b) in Phase 1b Dose Expansion/Optimization. The doses may be adjusted based on an SRC decision following review of the totality of the data.
c. Confirm documentation of a KRAS G12C mutation from local or central laboratory testing in tumor or liquid biopsy samples collected within 2 yr prior to screening. Note: Determination of KRAS G12C status by the central laboratory as confirmation of local laboratory testing is not required prior to enrollment.
D. For Phase 1a: patients with locally advanced or metastatic solid tumor and a KRAS-G12C mutation. For Phase 1b: patients with locally advanced or metastatic NSCLC and a KRAS-G12C mutation who are KRAS-G12Ci naïve.
E. Restaging scans (CT, MRI, or PET-CT) should be done at screening, C3D1 (±7 d) and then every 8 wk (±7 d) thereafter until PD. Scans are to be taken at the EOT Follow-up Visit unless taken within 28 d of the EOT Follow-up visit. Confirmatory scans will also be obtained at least 4 wk following initial documentation of an objective response (i.e., PR or CR per RECIST v1.1). The type of scan obtained is at the discretion of the investigator as appropriate for the disease. However, the same method should be used for the duration of the study. All scans will be read locally; in Phase 1b Dose Expansion/Optimization, scans will also be read via blinded independent central review. Patients who discontinue study drug for reasons other than radiographic PD will have radiographic assessments during LTFU per standard of care imaging, with data reported as they become available, until radiographic PD is observed (unless consent is withdrawn).
F. Relevant tumor markers will be assessed as appropriate using blood samples.
G. Eligibility to be confirmed prior to dosing.
H. Including year of birth, sex, height, race, ethnicity, as allowed per regional regulations.
I. Including relevant medical history, current medical conditions, oncology history (e.g., diagnosis, extent of cancer and stage, prior anticancer therapy), radiation history.
J. Complete physical examination to be performed at screening and EOT Follow-up Visit or ET (as applicable); focused examination (e.g., symptom directed per investigator's judgment) to be performed at other visits. Weight is to be included for both complete and focused examinations.
K. Vital signs should be taken before any blood draw.
L. SAEs should be collected from time of informed consent. Death solely due to progression of disease should not be reported as an SAE but as with all deaths, must be captured on the relevant CRF page.
M. Blood hematology, chemistry, coagulation, pregnancy, and urinalysis pre-dose assessments may be performed up to 2 d prior to scheduled visit. If these screening assessments are performed within 48 hr prior to Cycle 1 Day 1, these results may be used as baseline (pre-dose assessments) without requiring the pre-dose assessment within 2 hours of dosing as described in footnotes W, Y.
N. Women who are not surgically sterile to confirm menopausal state.
O. Women who are not surgically sterile or menopausal with serum test at all indicated time points. Patients must have a negative test within 48 hr of D1 of every cycle.
P. Complete blood count with differential.
Q. See Section 8.2.2 for the list of parameters in the clinical chemistry panel. [NOTE: patient should fast for all screening labs, all predose labs, and all labs up to 2 hours after dose]
R. Including ctDNA. Samples should be collected after at least 7 consecutive days of Compound (10b) dosing and may be excluded from sample collection if visit cannot be scheduled within allowable visit window.
S. Medical history and physical exam should be confirmed and updated with any findings which may have occurred prior to the first dose on C1D1.
T. Fresh tumor biopsies should be collected for all patients enrolled in the study if at all feasible. If fresh tumor biopsies were not done during molecular prescreening and are not feasible during Screening, archival tumor biopsies collected preferably within 6 mo but no later than 2 yr of enrollment and with sufficient tumor for central testing of KRAS mutations can be used. Patients may be enrolled even when tumor biopsies cannot be obtained at the discretion of the medical monitor, in consultation with the investigator. Enrollment of patients for whom collection of tumor biopsies will not occur will be handled on a case-by-case basis by the medical monitor. Please refer to the Lab Specimen Manual for details regarding collection and handling of tumor tissue samples.
U. If the start of the subsequent cycle is delayed due to simultaneous dose interruptions of both study medications, weekly assessments should be recorded as unscheduled visits to the just completed 28-d cycle until the dosing of at least one study treatment is resumed. The first day of the next cycle will be the day a study treatment is restarted. Should either Compound (10b) or sotorasib be interrupted for 15 consecutive days, the patient should be discontinued permanently from both study medications.
V. Cycle 1 Day 1 visit should take place on a Monday, Tuesday, Wednesday, or Thursday.
W. Collect pre-dose (within 2 hr prior to dosing study treatment[s]) and 4 hr (±15 min) after administration of study treatment(s).
X. Unless done in the prior 7 d.
Y Collect pre-dose within 2 hr prior to dosing of study treatment(s) on day of visit.
Z. Collect pre-dose (i.e., approximately 24 hr [±2 hr] after prior day's administration of study treatment(s) but before dosing of study treatment(s) on day of visit).
AA. For Compound (10b) and sotorasib PK: Collect blood samples pre-dose (within 2 hr prior to dosing of study treatment[s]) and 0.5 (±5 min), 1 (±10 min), 2 (±15 min), 4 (±15 min) and 6 hr (±15 min) after administration of study treatment(s). Steady-state (C2D1) PK samples should be collected after at least 7 consecutive days of dosing of each study treatment. All samples may be excluded from sample collection if visit cannot be scheduled within allowable visit window.
BB. If the patient will not be continuing on subsequent cycle (i.e., not continuing on study), the patient should still undergo all Day 1 visit procedures of the subsequent cycle, except the 2 hr PK and pharmacodynamic samples.
CC. Window (±3 d) allowed for each MUGA/ECHO evaluation starting at Cycle 2.
DD. Collect post-dose between 2 and 6 hr after administration of study treatment(s) on Cycle 2 Day 1 (±7 d).
EE. Collect pre-dose within 2 hr prior to and 2 hr (±15 min) after administration of study treatment(s) on day of visit. Samples should be collected after at least 7 consecutive days of dosing of study treatment(s). All samples may be excluded from collection if visit cannot be scheduled within allowable visit window.
FF. The 12-lead ECG will be taken in triplicate at screening and at C1D1, C2D1, C3D1, and every 2 cycles thereafter.
GG. Unless completed within 4 wk of the EOT Follow-up Visit.
HH. A MUGA scan or ECHO will be performed at the EOT Follow-up Visit and the ET visit only if an assessment of LVEF has not been performed within the last 14 d.
II. Patients who discontinue treatment for radiographic PD as well as patients who discontinue treatment for reasons other than PD and subsequently have radiographic PD will be followed approximately every 3 months (via telephone) for survival status and new anticancer therapy information until EOS. This survival follow-up also applies to patients who discontinue study treatment for reasons other than radiographic PD but withdraw consent for further tumor assessments. Survival follow-up will continue until all patients have discontinued study treatment, died, withdrawn consent, or been lost to follow-up, or until EOS, whichever occurs first. Long-term follow up will also include the collection of disease response(s) and secondary malignancies. Clinic visits may also take the place of long-term follow up telephone contact if scheduled as part of routine follow up.
JJ. C1D22 will be a telephone visit during which the daily self-administration of study treatment(s) will be reviewed, per the patient diary, and information on AEs and concomitant medications will be recorded in source documents and transcribed to the CRF.
KK. Only if there is no documentation of KRAS mutation within the 2 years prior to screening, molecular prescreening for KRAS mutation using an appropriate clinically validated and/or FDA approved test through a laboratory that is CLIA certified if applicable.
LL. Concomitant medications include all prescription and over-the-counter medication, vaccine, or dietary supplement (vitamins, minerals, herbs)
MM. For details on the pharmacodynamic assessments, refer to Section 8.5 of the clinical protocol.
NN. To qualify for the DLT assessment period, patients need to have taken 21 doses within the 28-day assessment period. See Section 4.1.2 and Section 9.1.1 of the clinical protocol for additional details.

TABLE 7
Planned Phase 1a Dose Escalation

| Cohorts (Number of DLT | Planned Doses^{b} | |
|---|---|---|
| Evaluable Patients)^{a} | Compound (10b) | Sotorasib |
| Cohort 1 (n = 5-7) | 250 mg PO QD | 960 mg PO QD |
| Cohort 2 (n = 5-7) | 400 mg PO QD | 960 mg PO QD |
| Cohort 3 (n = 5-7) | 550 mg PO QD | 960 mg PO QD |

Abbreviations: PO = by mouth; QD = once daily; RP1bD = recommended Phase 1b dose; SRC = Safety Review Committee
^{a}Additional patients may be enrolled in existing cohorts if deemed necessary by the SRC to evaluate the safety, tolerability, and the RP1bD.
^{b}Dose escalation, an additional dose level, alternative dose levels, and/or alternative dose schedules may be enrolled following SRC review of the totality of the data and after the SRC approves or makes a recommendation regarding the next dose cohort.

DLT is defined as an AE or abnormal laboratory value, excluding toxicities clearly related to disease progression or intercurrent illness, and occurring during the first cycle (28 days) on study that meets any of the following criteria:
Grade 3 laboratory abnormalities lasting more than 7 days or Grade 4 laboratory abnormalities of aspartate amino transferase/alanine amino transferase (AST/ALT) and/or bilirubin, of any duration, with the following exceptions:
   For patients with Grade 1 AST/ALT at baseline (>ULN to 2.5×ULN), an AST/ALT value of >7.5×ULN will be considered a DLT
Grade 3 or higher non-hematological toxicity excluding:
   Grade 3 nausea, vomiting or diarrhea for less than 72 hours with adequate supportive care
   Grade 3 fatigue lasting less than a week
   Grade 3 electrolyte abnormality that lasts for less than 72 hours that is not clinically complicated and resolves spontaneously or with conventional medical interventions
   Grade 3 amylase or lipase lasting less than 72 hours and not associated with manifestations of pancreatitis
Meet criteria for Hy's Law (i.e., ≥3×ULN ALT and/or AST with ≥2×ULN total bilirubin and alkaline phosphatase <2×ULN), unless patient has congenital hyperbilirubinemia.
Grade 3 neutropenia >7 days.
Grade 4 neutropenia.
Febrile neutropenia [ANC <1000/mm³ with single oral temperature >38.3° C. (101° F.) or with two consecutive oral temperatures ≥38° C. (100.4° F.) measured at least 1 hour apart].
Grade 3 thrombocytopenia with Grade 2 or higher bleeding.
Grade 4 thrombocytopenia or thrombocytopenia requiring platelet transfusion.
Grade 4 life-threatening anemia.
Grade 5 toxicities.
Any other ≥Grade 3 clinically significant or persistent toxicity that may be considered a DLT following review by the SRC.

### Adverse Events (AE)

An AE is the development of an undesirable medical condition or the deterioration of a pre-existing medical condition in a patient participating in a clinical study, whether or not it is considered to have a causal relationship with the study medication. An AE can therefore be any unfavorable and unintended sign (including an abnormal laboratory finding), symptom, or disease (new or exacerbated). In clinical studies, an AE can include an undesirable medical condition occurring at any time, including baseline or washout periods, even if no study treatment has been administered.

All AEs that occur after the patient has received the first dose of study medication, or during the study participation, whether or not they are related to study medication must be recorded on the appropriate form provided. Each AE is to be evaluated for seriousness, intensity, and causal relationship with each study medication or other factors. SAEs should be recorded and reported from time of informed consent.

At every visit, patients should be asked about AEs in an open-ended manner as well as asked about the status of any previously reported AEs.

When possible, a specific disease or syndrome rather than an individual associated sign or symptom should be identified by the investigator and recorded on the form. If an observed or reported sign or symptom is not considered a component of a specific disease or syndrome by the investigator, it should be recorded as a separate AE on the form.

NOTE: An unexpected worsening or exacerbation of the condition/indication under investigation should be reported as an AE. However, anticipated day-to-day fluctuations or expected progression of the disease under investigation (based upon the investigator's clinical judgment) are not to be considered AEs. Disease progression should be recorded on the relevant CRF page. Death solely due to progression of disease should not be reported as an SAE but as with all deaths, must be captured in the CRF.

Abnormal laboratory findings (e.g., clinical chemistry, hematology, and urinalysis), special investigations (e.g., scan, MUGA/ECHO) or other abnormal assessments (e.g., physical exam findings, ECGs, and vital signs) that are judged by the investigator as clinically significant i.e., requires further investigation and/or treatment will be recorded as AEs. Clinically significant abnormal laboratory findings, special investigations or other abnormal assessments that are detected during the study or are present at baseline and significantly worsen following exposure to study treatment are to be reported as AEs. However, clinically significant abnormal laboratory findings, special investigations or other abnormal assessments that are associated with the disease being studied, unless judged by the investigator as more severe than expected for the patient's condition, or that are present at the start of the study and do not worsen, should not be reported as AEs.

Medical or surgical procedures (e.g., endoscopy, appendectomy) are not AEs; the condition that necessitates the procedure is the AE if new or worsened from baseline. Elective procedures or preplanned interventions for preexisting conditions that did not worsen since the start of the study are also not AEs; the preexisting condition should be captured as medical history. Situations in which an untoward medical occurrence did not occur but hospitalization is for social reasons and/or convenience are not AEs.

It is the responsibility of the investigator to review all laboratory findings, including laboratory assessments performed at outside labs, in all patients and determine if they constitute an AE. The investigator will assess external laboratory findings and determine clinical significance of any which are deemed AEs. In these instances, the laboratory reports should be signed and dated by the investigator.

### Serious Adverse Events (SAEs)

An SAE is any AE, occurring at any dose and regardless of causality, that:
Results in death or death is the outcome of an AE and not the AE itself unless the cause of death is unknown in which case death should be recorded as the AE.
Is life-threatening
   Life-threatening means that, in the opinion of the investigator or study sponsor, the patient was at immediate risk of death from the reaction as it occurred, (i.e., it does not include a reaction that hypothetically might have caused death had it occurred in a more severe form).
Requires in patient hospitalization or prolongation of existing hospitalization.
   In general, hospitalization signifies that the patient has been detained (usually involving at least an overnight stay) at the hospital or emergency ward for observation and/or treatment that would not have been appropriate in the physician's office or outpatient setting.
   Complications that occur during hospitalization are AEs. If a complication prolongs hospitalization or fulfills any other serious criteria, the event is serious.
   Hospitalization admissions and/or surgical operations scheduled to occur during the study period, but planned before the signing of the ICF, are not considered AEs if the illness or disease existed before the patient was enrolled in the trial, provided that it did not deteriorate in an unexpected manner during the trial (e.g., surgery performed earlier than planned).
Results in persistent or significant disability/incapacity.
   Disability is defined as a substantial disruption of a person's ability to conduct normal life functions.
   This definition is not intended to include experiences of relatively minor medical significance such as uncomplicated headache, nausea, vomiting, diarrhea, influenza, and accidental trauma (e.g., sprained ankle) that may interfere with or prevent everyday life functions but do not constitute a substantial disruption.
Is a congenital anomaly/birth defect.
Is an important medical event.
   An important medical event is an event that may not result in death, be life-threatening, or require hospitalization but may be considered an SAE when, based upon appropriate medical judgment, it may jeopardize the patient and may require medical or surgical intervention to prevent one of the outcomes listed in the definitions for SAEs. Examples of such medical events include allergic bronchospasm requiring intensive treatment in an emergency room or at home, blood dyscrasias or convulsions that do not result in in patient hospitalization, or the development of drug dependency or drug abuse.

A distinction should be made between the terms “serious” and “severe” since they are not synonymous. The term “severe” is often used to describe the intensity (severity) of a specific event (as in mild, moderate, or severe myocardial infarction); the event itself, however, may be of relatively minor medical significance (such as severe headache). This is not the same as “serious,” which is based on patient/event outcome or action criteria usually associated with events that pose a threat to a patient's life or functioning. A severe AE does not necessarily need to be considered serious. For example, persistent nausea of several hours' duration may be considered severe nausea but not an SAE if the event does not meet the serious criteria. On the other hand, a stroke resulting in only a minor degree of disability may be considered mild but would be defined as an SAE based on the above noted serious criteria. Seriousness (not severity) serves as a guide for defining regulatory reporting obligations.

Intensity of AEs are graded using NCI CTCAE v 5.0, accessed at https://ctep.cancer.gov/protocolDevelopment/electronic_applications/docs/CTCAE_v5_Quick_Reference_5×7.pdf, the disclosure of which is incorporated by reference in its entirety.

### Administration, Dosing Adjustments, Delays, and Discontinuations

Each treatment cycle will be 28 days in duration unless alternative dosing schedules are implemented based on SRC review. Absent a reason to interrupt dosing, a new cycle will be initiated immediately upon completion of the prior cycle. Compound (10b) and sotorasib will be self-administered with no interruptions unless 1) there are AEs, as described below; 2) the SRC recommends an alternative dosing schedule based on the safety, tolerability, and PK/pharmacodynamic data observed; or 3) patients are seen in the clinic for assessment of AEs, PK sample collection, or laboratory evaluations. At such visits, the patient will take Compound (10b) and sotorasib concurrently at the clinic as instructed by research staff. For any blood draws performed outside of the clinic, the patient will take Compound (10b) and sotorasib and record/provide dosing information as instructed by research staff. Each dose of Compound (10b) should be taken once orally every 24 hours (±3 hours) with approximately 240 mL (8 ounces) of water. Patients should take Compound (10b) after an overnight fast (minimum 8 hours) followed by 2 hours of fasting after the dose is taken. Water is allowed ad lib except 1 hour before and after dosing. If a dose is missed, the next dose will not be increased to account for missing a dose. The patient will take the next regular dose at the scheduled time.

Although sotorasib could be taken with or without food, the 2 study treatments should be taken in a fasted state as described for Compound (10b). Compound (10b) should be taken before sotorasib. The day before and the day of study visit, both study treatments should be taken within a 5-minute window.

If either Compound (10b) or sotorasib is permanently discontinued, the patient must be discontinued from the other study treatment as well; monotherapy with Compound (10b) or sotorasib is not allowed in this study.

Reasons for permanently discontinuing both study treatments (Compound (10b) and sotorasib) include, but are not limited to, the following:
Patient develops a DLT and cannot be continued on a lower dose of study treatment(s) (as discussed elsewhere in this example);
Either study treatment is permanently discontinued for reasons as outlined elsewhere for either Compound (10b) or sotorasib;
Disease progression according to RECIST v.1.1 per investigator assessment;
Risk to patient, as judged by the investigator and/or sponsor;
Severe noncompliance with the protocol, as judged by the investigator and/or sponsor;
Patient becomes pregnant;
Patient's decision;
Start of a non-study anticancer treatment; and
Does not restart treatment within 15 days of dose interruption

Specific reasons for withdrawal from the study will include the following:
Withdrawal of consent by the patient, who is at any time free to withdraw from participation in the study, without prejudice to further treatment;
Loss to follow-up (defined as no contact after 3 documented attempts by telephone followed by 1 attempt via certified letter);
Death from any cause;
Sponsor's decision to terminate study; and
Investigator's decision.

For each patient, a maximum of two dose reductions per oral agent (Compound (10b) or sotorasib) will be allowed. Dose reduction for each oral agent is discussed in detail elsewhere in this example.

### Compound (10b)

Compound (10b) dose modification decisions should be based on the CTCAE grading scale v5.0 and according to Table 8. If drug-related AEs are not specified in the table, doses may be reduced or held or permanently discontinued at the discretion of the investigator for the patient's safety.

For each patient, a maximum of two dose reductions of Compound (10b) will be allowed. Dose reduction for Compound (10b) means treatment at the next lower, previously tested dose level as outlined in Table 7. If a dose reduction is required below the lowest Compound (10b) dose previously tested, the dose should be selected after discussion between the investigator and the medical monitor.

TABLE 8
Dose Adjustments of Compound (10b)
for Drug-Related Adverse Events

| Grade | Occurrence | Dose Modification^{a} |
|---|---|---|
| 1 or 2 | Any time | No dose reduction. |
| 3 | 1st and 2nd | Hold treatment with Compound (10b). |
| | time | If the drug-related AE resolves to ≤ Grade 1 or |
| | | baseline ≤14 days, resume Compound (10b) at |
| | | the next lower dose level as outlined in Table 2 |
| | | and previously tested and SRC approved. Dose |
| | | re-escalation to the dose of Compound (10b) at |
| | | which the drug-related AE occurred, is not |
| | | permitted. |
| | | If the drug-related AE persists for >14 days, |
| | | discontinue patient from Compound (10b) |
| | | permanently. |
| | 3rd time | Discontinue patient from Compound (10b) |
| | | permanently. |
| | | (For patients with clinical benefit/response from |
| | | Compound (10b) continuation may be considered |
| | | if toxicities resolve to ≤ Grade 1 and with proper |
| | | dose adjustments after consultation between the |
| | | investigator and the medical monitor). |
| 4 | Any time | Discontinue patient from Compound (10b) |
| | | permanently. |
| | | (For patients with clinical benefit/response from |
| | | Compound (10b) continuation may be considered |
| | | if toxicities resolve to ≤ Grade 1 and with proper |
| | | dose adjustments after consultation between the |
| | | investigator and the medical monitor). |

^{a}These general guidelines constitute guidance to the investigator and may be supplemented by discussions with the medical monitor in specific cases.

### Other Allowable Compound (10b) Dose Modifications

Treatment interruptions and dose modifications other than the ones mentioned above can be considered for Compound (10b) after discussion between the investigator and the medical monitor and proper documentation of the rationale.

Any changes should be within the following guidelines:
Further dose reductions of Compound (10b) can be made to keep clinically significant drug-related AEs Grade <1. Dose adjustments by more than 1 dose level at a time can be considered when judged in the best interest of the patient (e.g., severe myelosuppression, lactic acidosis) when the toxicity has resolved.
If a Grade 3 AE considered related to Compound (10b) is observed, treatment may be delayed for up to 2 weeks to allow resolution of the toxicity, with re-treatment at a reduced dose at the discretion of the investigator. If Grade 3 nausea, emesis, diarrhea, or clinically significant electrolyte abnormalities occur and resolve within 3 days on optimum treatment, treatment may be resumed without dose reduction. If recurrent Grade 3 nausea, emesis, diarrhea, or clinically significant electrolyte abnormalities occur of >3 days occur, treatment can be resumed with dose reduction after resolution of symptoms.
If a patient experiences more than one AE, Compound (10b) dose decisions will be based on the most severe AE.
A patient who requires a dose reduction and subsequently experiences a recurrent, clinically significant drug-related AE may undergo one additional dose reduction. Patients who continue to experience the event after two dose reductions will be withdrawn from the study. The lower doses will be based first on the lower doses cleared by the SRC as part of the dose escalation of this study and if lower doses are required, based on SRC cleared doses in Study NAV-1001.

### Sotorasib

Per the product label for LUMAKRAS (LUMAKRAS Package Insert 2021, incorporated by reference in its entirety herein), two dose reductions are allowed for sotorasib in the event of an adverse reaction (reduction to 480 mg per day; reduction to 240 mg per day). Drug should be discontinued if patients are unable to tolerate the minimum dose of 240 mg per day.

Dosage modifications for sotorasib due to adverse reactions are presented in Table 9.

TABLE 9
Recommended Dose Modifications for
Sotorasib due to Adverse Reactions

| Adverse Reaction | Severity | Dosage Modification |
|---|---|---|
| Hepatotoxicity | Grade 2 AST or | Withhold sotorasib until |
| | ALT with | recovery to ≤ Grade 1 or |
| | symptoms, Or | baseline. |
| | Grade 3 to 4 AST | Resume sotorasib at the next |
| | or ALT | lower dose level. |
| | AST or ALT >3 × | Permanently discontinue |
| | ULN with total | sotorasib. |
| | bilirubin >2 × ULN | |
| | in the absence of | |
| | alternative causes | |
| ILD/pneumonitis | Any Grade | Withhold sotorasib if |
| | | ILD/pneumonitis is |
| | | suspected. |
| | | Permanently discontinue |
| | | sotorasib if ILD/pneumonitis |
| | | is confirmed. |
| Nausea or vomiting | Grade 3 to 4 | Withhold sotorasib until |
| despite appropriate | | recovery to ≤ Grade 1 or |
| supportive care | | baseline. |
| (including anti- | | Resume sotorasib at the next |
| emetic therapy) | | lower dose level. |
| Diarrhea despite | Grade 3 to 4 | Withhold sotorasib until |
| appropriate | | recovery to ≤ Grade 1 or |
| supportive care | | baseline. |
| (including | | Resume sotorasib at the next |
| antidiarrheal | | lower dose level. |
| therapy) | | |
| Other adverse | Grade 3 to 4 | Withhold sotorasib until |
| reactions | | recovery to ≤ Grade 1 or |
| | | baseline. |
| | | Resume sotorasib at the next |
| | | lower dose level. |

Abbreviations: ALT = alanine aminotransferase, AST = aspartate aminotransferase, CTCAE = Common Terminology Criteria for Adverse Events; ILD = interstitial lung disease, ULN = upper limit normal
Note:
Grading is based on the CTCAE v5.0.
Source: Product label LUMAKRAS (LUMAKRAS Package Insert 2021, incorporated by reference in its entirety)

### Efficacy Assessments

Restaging scans (computed tomography [CT], MRI, or positron emission tomography/CT[PET-CT]) will be performed. Confirmatory scans should be obtained at least 4 weeks following initial documentation of an objective response (i.e., PR or CR per RECIST v1.1). The type of scan obtained is at the discretion of the investigator as appropriate for the disease. However, the same method should be used for the duration of the study.

Patients who discontinue treatment for reasons other than radiographic PD will continue to have radiographic assessments per standard of care imaging, with data reported as they become available, or the initiation of subsequent anticancer therapies, death, or patient withdraws consent for radiographic assessments, whichever occurs first.

Tumor response will be evaluated using RECIST v1.1 (Eisenhauer Eur J Cancer; 2009; 45(2):228-247). Tumor response will be evaluated locally by the investigator for the study; scans for the Phase 1b portion of the study will also be read via blinded independent central review (BICR). Progression free survival will be determined based upon date of first documented progression or death due to any cause.

### Pharmacokinetics (PK) and Pharmacodynamics (PD)

### Pharmacokinetics

Blood samples for PK evaluation of Compound (10b) and sotorasib will be collected from all enrolled patients participating in the study. Time points of blood sample collection for PK of Compound (10b) and sotorasib are outlined in Table 10. Additional details on collection and storage of samples can be found in the Laboratory Manual.

TABLE 10
Compound (10b) and Sotorasib PK Sample Collection Schedule

| | | Time Points | | | | | |
|---|---|---|---|---|---|---|---|
| | | Pre-dose | | | | | |
| | | (within 2 hr | 0.5 hour | 1 hour | 2 hour | 4 hour | 6 hour |
| | | prior to Comp. | post-dose | post-dose | post-dose | post-dose | post-dose |
| Cycle Day | | (10b) dosing) | (±5 min) | (±10 min) | (±15 min) | (±15 min) | (±15 min) |
| 1 | 1 | X | X | X | X | X | X |
| 1 | 2 | X^{A} | | | | | |
| 1 | 8 | X | | | | | |
| 1 | 15 | X | | | | | |
| 2 | 1 | X | X | X | X | X | X |
| ≥3 | 1 | X | | | X | | |

^{A}Collect pre-dose (i.e., approximately 24 hr [±2 hr] after prior day's administration of study treatment(s) but before dosing of study treatment(s) on day of visit).

On the days of PK sampling, patients should take the study medications at the clinic as instructed by research staff. Patients who forget and take their medication at home will be excluded from PK analysis for that day; they should not have blood samples collected. Complete dosing information, including the date and time of actual blood draw and time of the last study treatment dose prior to the sampling, should be obtained on all sampling days and recorded on the appropriate blood collection CRF. If any of the scheduled sampling times are missed or a sample is not drawn according to this schedule, the actual collection date and time will be recorded, and the remaining samples will be collected on schedule whenever possible.

Steady-state (C2D1 and beyond) PK samples should be collected after at least 7 consecutive days of dosing of each study treatment. If a study visit cannot be rescheduled within an acceptable window, PK samples should not be drawn.

If vomiting occurs within 4 hours following Compound (10b) or sotorasib administration on the day of PK blood sampling, the time (using a 24-hour clock) of vomiting should be recorded. Additional Compound (10b) or sotorasib should not be taken in an effort to replace the material that has been vomited. The occurrence and frequency of any vomiting during a treatment cycle must be noted in the AE CRF. PK collection should continue as scheduled in all cohorts.

### Pharmacodynamics

Blood and tumor samples will be used to assess the pharmacodynamic effects of Compound (10b) in combination with sotorasib and potential mechanisms of antitumor response and resistance, including but not limited to assessments provided in Table 11. These samples will also be used to develop biomarkers that may be used to predict response or resistance to Compound (10b) in combination with sotorasib.

Fresh tumor biopsies should be collected for all patients enrolled in the study if at all feasible. If fresh tumor biopsies were not done during molecular prescreening and are not feasible during screening, archival tumor biopsies collected preferably within 6 months but no later than 2 years of enrollment with sufficient tumor for central testing of KRAS G12C mutation can be used. Fine needle aspirates or other cytology samples are not acceptable.

Patients may be enrolled even when tumor biopsies cannot be obtained at the discretion of the medical monitor, in consultation with the investigator. Enrollment of patients for whom collection of tumor biopsies will not occur will be handled on a case-by-case basis by the medical monitor.

The samples obtained will be used in different biomarker studies, including:
Gene expression within biopsied tumor tissue and circulating in the blood to identify genes or a signature of genes whose expression is modulated by Compound (10b), and which may serve as pharmacodynamic biomarkers.
Molecular profiling (e.g., NGS, PCR, etc.) of tumor biopsies and ctDNA (liquid biopsies) to evaluate potential mechanisms of drug resistance.
pSHP2 and pERK assessment via immunohistochemistry in tumor tissue as a measure of MAPK pathway flux.
Tumor microenvironment (TME) profiling in tumor biopsies via IHC or multiplex immunofluorescence as a measure of tumor immune cell infiltration and activation.

TABLE 11
Pharmacodynamic Assessments

| | | | | | Sub- | |
|---|---|---|---|---|---|---|
| | Screen- | Cycle | | Cycle | sequent | |
| | ing | 1 | | 2 | Cycles | EOT V1 |
| Study Day | −30 | 1 | 2 | 1 | 1 | Last |
| | to −1 | | | | | dose + |
| | | | | | | 28 days |
| +/− days allowable | 0 | 0 | 0 | ±3 | ±3 | ±3 |

Tumor biopsy

| | | | |
|---|---|---|---|
| pSHP2 IHC | X | X | X |
| pERK IHC | X | X | X |
| TME biomarkers | X | X | X |
| IHC/IF | | | |
| Transcriptomics | X | X | X |

Blood

| | | | | |
|---|---|---|---|---|
| ctDNA NGS | X | X | X^{A} | X |

^{A}Samples should be collected after at least 7 consecutive days of Compound (10b) dosing

### BOIN Design

A BOIN design with target toxicity rate of ϕ=0.25 will be used to find the MTD of Compound (10b) in combination with sotorasib. As shown in FIG. 6, the BOIN design uses the following rule, optimized to minimize the probability of incorrect dose assignment, to guide dose escalation/de-escalation:
if the observed DLT rate at the current dose is ≤0.197, escalate the dose to the next higher dose level;
if it is ≥0.298, de-escalate the dose to the next lower dose level;
otherwise, stay at the current dose.

For the purpose of overdose control, doses j and higher levels will be eliminated from further examination if Pr(pⱼ>0.25|data) >0.95, where pⱼ is the true DLT rate of dose level j, j=1, . . . , 3. This posterior probability is evaluated based on the beta-binomial model yⱼ|pⱼ˜binomial(pⱼ) with pⱼ˜uniform(0, 1), where yⱼ is the number of patients experienced DLT at dose level j. When the lowest dose is eliminated, stop the trial for safety. The probability cutoff 0.95 is chosen to be consistent with the common practice that when the target DLT rate Φ≤⅙, a dose with ⅗ patients experiencing DLT is eliminated.

The steps to implement the BOIN design are described as follows:
1. To assign a dose to the next cohort of patients (which will be done by the SRC), dose escalation/de-escalation will be conducted according to the rules displayed in Table 12 and based on a target toxicity rate of 25%. When using Table 12, please note the following:
   a. “Eliminate” means eliminate the current and higher doses from the trial to prevent treating any future patients at these doses because they are overly toxic.
   b. When a dose is eliminated, automatically de-escalate the dose to the next lower level. When the lowest dose is eliminated, the trial will be stopped for safety. In this case, no dose should be selected as the MTD.
   c. If none of the actions (i.e., escalation, de-escalation, or elimination) are triggered, new patients will be treated at the current dose.
   d. If the current dose is the lowest dose and the rule indicates dose de-escalation, new patients will be treated at the lowest dose unless the number of DLTs reaches the elimination boundary, at which point the trial will be terminated for safety.
   e. If the current dose is the highest dose and the rule indicates dose escalation, new patients will be treated at the highest dose.
   f. In the event that operational/practical circumstances result in an over-enrollment (i.e., >7 DLT evaluable patients per the current cohort size), the next dose level decision would be based on the actual number of DLT evaluable patients in the cohort.
2. Dose escalation will start with 5-7 DLT evaluable patients being enrolled at Compound (10b) 250 mg plus sotorasib 960 mg (i.e., the lowest dose level). Per Table 12, for illustrative purposes, if one DLT is observed in any of 6 evaluable patients enrolled, escalate to the next dose level. If a DLT is observed in ≥2 of 6 evaluable patients enrolled, then de-escalate to a lower dose.
3. If escalated to a higher dose level, follow same approach as in 2.
4. The maximum sample size for dose escalation is 21 DLT evaluable patients and 10 DLT evaluable in any dose level cohort.

TABLE 12
Dose Escalation Decision Rules

The number of patients treated
at the current dose

| Actions ^{a} | 5 | 6 | 7 | 8 | 9 | 10 |
|---|---|---|---|---|---|---|
| Escalate if # of | 0 | 1 | 1 | 1 | 1 | 1 |
| patients with DLT ≤ | | | | | | |
| De-escalate if # of | 2 | 2 | 3 | 3 | 3 | 3 |
| patients with DLT ≥ | | | | | | |
| Eliminate if # of | 3 | 4 | 4 | 5 | 5 | 5 |
| patients with DLT ≥ | | | | | | |

Abbreviations: DLT = dose limiting toxicity
^{a} When none of the actions (i.e., escalate, de-escalate, or eliminate) are triggered, stay at the current dose for treating the next cohort of patients. Note that “# of DLT” is the number of patients with at least 1 DLT.

After the trial is completed, the MTD will be selected based on isotonic regression as specified in Liu and Yuan (2015). This computation is implemented by the “Estimate MTD” tab of the BOIN Design Desktop Program. Specifically, select as the MTD the dose for which the isotonic estimate of the DLT rate is closest to the target DLT rate. If there are ties, select the higher dose level when the isotonic estimate is lower than the target DLT rate and select the lower dose level when the isotonic estimate is greater than or equal to the target DLT rate. The RP1bD for cohort expansion will be chosen based on toxicity (i.e., MTD), as well as other clinical considerations, e.g., PK/pharmacodynamics.

Table 13 shows the operating characteristics of the trial design based on 1000 simulations of the trial using the BOIN Design Desktop Program (MD Anderson Cancer Center, 2021). The operating characteristics show that the design selects the true MTD, if any, with high probability and allocates more patients to the dose levels with the DLT rate closest to the target of 0.3.

TABLE 13
Operating Characteristics for BOIN
Dose Finding from Simulation

| | Dose^{a} Level of | | | | |
|---|---|---|---|---|---|
| | Compound (10b) | | | Number of | % Early |
| | 1 | 2 | 3 | Patients | Stopping |
| Scenario 1 | | | | | |
| True DLT Rate | 0.02 | 0.03 | 0.04 | | |
| Selection % | 0.6 | 3.2 | 96.2 | | 0.0 |
| % Pts Treated | 27.1 | 28.7 | 44.2 | 20 | |
| Scenario 2 | | | | | |
| True DLT Rate | 0.03 | 0.06 | 0.08 | | |
| Selection % | 2.8 | 9.5 | 87.7 | | 0.0 |
| % Pts Treated | 30.1 | 31.5 | 38.3 | 20 | |
| Scenario 3 | | | | | |
| True DLT Rate | 0.06 | 0.11 | 0.16 | | |
| Selection % | 11.6 | 29.9 | 58.4 | | 0.1 |
| % Pts Treated | 36.8 | 35.7 | 27.5 | 19 | |
| Scenario 4 | | | | | |
| True DLT Rate | 0.13 | 0.22 | 0.32 | | |
| Selection % | 39.8 | 40.3 | 17.8 | | 2.1 |
| % Pts Treated | 52.8 | 35.6 | 11.6 | 17 | |
| Scenario 5 | | | | | |
| True DLT Rate | 0.25 | 0.44 | 0.64 | | |
| Selection % | 78.0 | 9.0 | 0.1 | | 12.9 |
| % Pts Treated | 79.3 | 19.9 | 0.8 | 13 | |

Abbreviations: BOIN = Bayesian optimal interval design; DL = the highest dose cleared by the SRC in the ongoing Compound (10b) monotherapy trial (NCT04528836); DLT = dose limiting toxicity
^{a}Dose escalation, alternative dose levels, and/or alternative dose schedules will proceed following review of the totality of the safety data by the SRC and after the SRC approves or makes a recommendation regarding the next dose cohort.
Note:
“% Early Stopping” refers to early stopping due to excessive DLT.

### Efficacy

### Patient Evaluation & Statistics

Disease assessment will be performed in patients with solid tumors at screening, then every 8 weeks thereafter, unless more frequent assessments are warranted clinically or per RECIST v1.1 criteria outlined herein.

Descriptive statistics will be provided for continuous variables, and frequency tables will be used to summarize categorical variables. The best overall response and the proportions of patients who experienced CR, PR, SD and PD in patients with measurable disease will be displayed along with the corresponding 95% exact CIs.

Time-to-event variables (e.g., DOR, PFS, and OS) will be analyzed by the Kaplan-Meier method. Median, along with 25^{th} percentile, 75^{th} percentile, the minimum, and maximum, time to event will be estimated, and Kaplan-Meier curves will be plotted.

### Antitumor Effect—Solid Tumors

Tumor response is the primary efficacy endpoint in Phase 1b Dose Expansion/Optimization; patients will be assessed using standard criteria (Note: all patients must have measurable disease at study entry and therefore will be assessed per RECIST v1.1 criteria). For the purposes of this study, tumor response should be assessed every 8 weeks (±7 days) (i.e., Day 1 of every odd cycle) thereafter until PD while patients are on treatment. Disease assessments for patients who discontinue for reasons other than PD are outlined in section 7.6 of the clinical protocol. In addition to a baseline scan, confirmatory scans will also be obtained at least 4 weeks following initial documentation of an objective response (i.e., PR or CR).

Response and progression will be evaluated in this study using RECIST v1.1; the new international criteria proposed by the RECIST Committee (Eisenhauer et al., Eur J Cancer; 2009; 45(2):228-247). Changes in only the largest diameter (unidimensional measurement) of the tumor lesions are used in the RECIST criteria.

### Disease Parameters

Measurable disease. Measurable lesions are defined as those that can be accurately measured in at least one dimension (longest diameter [LD] to be recorded) as ≥10 mm by scan (CT, MRI, or PET-CT; scan slice thickness should be no greater than 5 mm). All tumor measurements must be recorded in millimeters (or decimal fractions of centimeters).

Non-measurable disease. All other lesions (or sites of disease), including small lesions (LD<10 mm or pathological lymph nodes with ≥10 mm to <15 mm) are considered non-measurable disease. Bone lesions, leptomeningeal disease, ascites, pleural/pericardial effusions, lymphangitis cutis/pulmonis, inflammatory breast disease, abdominal masses (not followed by CT, MRI, or PET-CT), and cystic lesions are all truly non-measurable.

Target lesions. All measurable lesions up to a maximum of 2 lesions per organ and 5 lesions in total, representative of all involved organs, should be identified as target lesions and recorded and measured at baseline. Target lesions should be selected on the basis of their size (lesions with the LD) and their suitability for accurate repeated measurements (either by imaging techniques or clinically). A sum of the LD for all target lesions will be calculated and reported as the baseline sum LD. The baseline sum LD will be used as reference by which to characterize the objective tumor response.

Non-target lesions. All other lesions (or sites of disease) including any measurable lesions over and above the 5 target lesions should be identified as non-target lesions and should also be recorded at baseline. Measurements of these lesions are not required, but the presence or absence of each should be noted throughout follow-up.

### Methods for Evaluation of Measurable Disease

All measurements should be taken and recorded in metric notation using a ruler or calipers. All baseline evaluations should be performed as closely as possible to the beginning of treatment and never more than 4 weeks before the beginning of the treatment.

The same method of assessment and the same technique should be used to characterize each identified and reported lesion at baseline and during follow-up. Imaging-based evaluation is preferred to evaluation by clinical examination when both methods have been used to assess the antitumor effect of a treatment.

CT, MRI, or PET-CT scans should be used to measure lesions selected for response assessment: These techniques should be performed with cuts of 5 mm or less in slice thickness contiguously. The same method of assessment and the same technique should be used to characterize each identified and reported lesion at baseline and during follow-up.

Tumor markers: Tumor markers alone cannot be used to assess response. If markers are initially above the upper normal limit, they must normalize for a patient to be considered in complete clinical response. Specific additional criteria for standardized usage of prostate-specific antigen and CA-125 response in support of clinical trials are being developed.

Cytology, Histology: These techniques can be used to differentiate between PR and CR in rare cases (e.g., residual lesions in tumor types, such as germ cell tumors, where known residual benign tumors can remain).

The cytological confirmation of the neoplastic origin of any effusion that appears or worsens during treatment when the measurable tumor has met criteria for response or stable disease is mandatory to differentiate between response or stable disease (an effusion may be a side effect of the treatment) and PD.

### Response Criteria

### Evaluation of Target Lesions

Complete Response (CR): Disappearance of all target lesions.

Partial Response (PR): At least a 30% decrease in the sum of the LD of target lesions, taking as reference the baseline sum LD.

Progressive Disease (PD): At least a 20% increase in the sum of the LD of target lesions, taking as reference the smallest sum LD recorded since the treatment started or the appearance of one or more new lesions.

Stable Disease (SD): Neither sufficient shrinkage to qualify for PR nor sufficient increase to qualify for PD, taking as reference the smallest sum LD since the treatment started.

### Evaluation of Non-Target Lesions

Complete Response (CR): Disappearance of all non-target lesions and normalization of tumor marker level.

Note: If tumor markers are initially above the upper normal limit, they must normalize for a patient to be considered in complete clinical response.

Stable Disease (SD): Persistence of one or more non-target lesion(s) and/or maintenance of tumor marker level above the normal limits

Progressive Disease (PD): Appearance of one or more new lesions and/or unequivocal progression of existing non-target lesions

Although a clear progression of “non-target” lesions only is exceptional, the opinion of the treating physician should prevail in such circumstances, and the progression status should be confirmed at a later time by the review panel (or principal investigator).

### Evaluation of Best Overall Response

The best overall response is the best response recorded from the start of the treatment until disease progression/recurrence (taking as reference for PD the smallest measurements recorded since the treatment started). The patient's best response assignment will depend on the achievement of both measurement and confirmation criteria.

TABLE 14
Evaluation of Best Overall Response

| | | | | Best Response |
|---|---|---|---|---|
| Target | Non-Target | New | Overall | for this Category |
| Lesions | Lesions | Lesions | Response | Also Requires: |
| CR | CR | No | CR | ≥4 wk confirmation |
| CR | Non-CR/ | No | PR | ≥4 wk confirmation |
| | Non-PD | | | |
| PR | Non-PD | No | PR | |
| SD | Non-PD | No | SD | Documented at least |
| | | | | once ≥4 wk from |
| | | | | baseline |
| PD | Any | Yes or No | PD | No prior SD, PR |
| Any | PDª | Yes or No | PD | or CR |
| Any | Any | Yes | PD | |

^{a}In exceptional circumstances, unequivocal progression in non-target lesions may be accepted as disease progression.
Note:
Patients with a global deterioration of health status requiring discontinuation of treatment without objective evidence of disease progression at that time should be reported as “symptomatic deterioration”. Every effort should be made to document the objective progression even after discontinuation of treatment.

### Duration of Response

Duration of overall response: DOR is measured from the time measurement criteria are met for CR or PR (whichever is first recorded) until the first date that recurrent or PD is objectively documented (taking as reference for PD the smallest measurements recorded since the treatment started).

The duration of overall CR is measured from the time measurement criteria are first met for CR until the first date that recurrent disease is objectively documented.

Duration of stable disease: Stable disease is measured from the start of the treatment until the criteria for progression are met, taking as reference the smallest measurements recorded since the treatment started.

### Progression Free Survival

Progression free survival (PFS) is defined as the date of the start of treatment to the date of the event defined as the first documented progression or death due to any cause. If patient has not had an event, PFS will be censored according to details described in the SAP. Kaplan-Meier analysis of PFS will be provided.

Although the foregoing disclosure has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of treating cancer in a subject, comprising administering to the subject:
a) a therapeutically effective amount of a compound represented by formula (I): 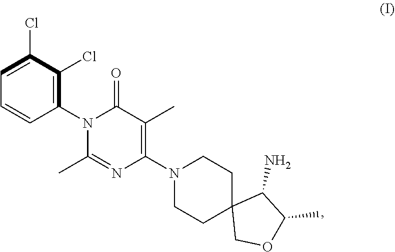
or a pharmaceutically acceptable salt, hydrate, solvate, stereoisomer, conformational isomer, tautomer, or a combination thereof; and
b) a therapeutically effective amount of sotorasib (AMG 510).

2. The method of claim 1, wherein the compound of formula (I) is represented by formula (10b): 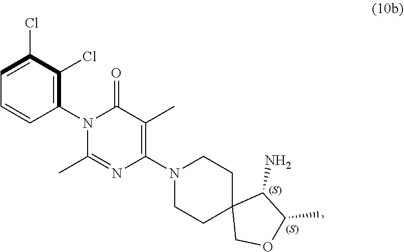
having the name of 6-((3S,4S)-4-amino-3-methyl-2-oxa-8-azaspiro[4.5]decan-8-yl)-3-(Ra)-(2,3-dichlorophenyl)-2,5-dimethylpyrimidin-4(3H)-one.

3. The method of claim 1 or 2, wherein the cancer is characterized by a KRAS mutation.

4. The method of claim 3, wherein the cancer is characterized by a KRAS G12C mutation.

5. The method of any one of claims 1 to 4, wherein the cancer comprises a solid tumor.

6. The method of any one of claims 1 to 5, wherein the cancer is lung cancer, colorectal cancer, pancreatic cancer, urothelial carcinoma, stomach cancer, mesothelioma, or a combination thereof.

7. The method of claim 6, wherein the cancer is non-small cell lung cancer (NSCLC).

8. The method of any one of claims 1 to 7, wherein the cancer is a KRAS G12C-positive cancer resistant to a KRAS G12C inhibitor.

9. The method of any one of claims 1 to 8, wherein the cancer is a KRAS G12C-positive cancer characterized by intrinsic and/or acquired resistance to a KRAS G12C inhibitor.

10. The method of any one of claims 1 to 9, wherein the cancer is a KRAS G12C-positive cancer resistant to sotorasib.

11. The method of any one of claims 1 to 10, wherein the cancer has progressed or recurred on or after at least one prior line of a systemic therapy comprising a platinum-based doublet chemotherapy and/or an anti-PD-1/PD-L1 therapy, each of which is given in monotherapy or both of which are given in combination therapy.

12. The method of any one of claims 1 to 11, wherein the subject does not have an activating mutation in BRAF V600X, PTPN11 (SHP2), or KRAS Q61X.

13. The method of any one of claims 1 to 12, wherein the subject is not previously treated with a PTPN11 inhibitor.

14. The method of any one of claims 1 to 12, wherein the subject is previously treated with a PTPN11 inhibitor other than a compound of formula (I).

15. The method of any one of claims 1 to 12, wherein the subject is previously treated with a compound of formula (I).

16. The method of any one of claims 1 to 15, wherein the subject is not previously treated with a KRAS G12C inhibitor.

17. The method of any one of claims 1 to 8 and 10 to 15, wherein the subject is previously treated with a KRAS G12C inhibitor.

18. The method of any one of claims 1 to 15, wherein the subject is previously treated with sotorasib.

19. The method of any one of claims 1 to 18, wherein the subject meets all of inclusion criteria of 1) to 11) according to Example 3, provided that the subject does not meet any one of exclusion criteria of 1) to 17) according to Example 3.

20. The method of any one of claims 1 to 19, wherein the subject is human.

21. The method of any one of claims 1 to 20, wherein the compound of formula (I) or (10b) and sotorasib are administered concomitantly.

22. The method of any one of claims 1 to 20, wherein the compound of formula (I) or (10b) and sotorasib are administered sequentially.

23. The method of claim 21, wherein the compound of formula (I) or (10b) is administered prior to the administration of sotorasib.

24. The method of claim 21, wherein the compound of formula (I) or (10b) is administered after the administration of sotorasib.

25. The method of any one of claims 1 to 24, wherein the compound of formula (I) or (10b) or sotorasib are administered orally, or each of sotorasib and the compound of formula (I) or (10b) are administered orally.

26. The method of any one of claims 1 to 25, wherein the compound of formula (I) or (10b) is administered orally.

27. The method of any one of claims 1 to 26, wherein sotorasib is administered orally.

28. The method of any one of claims 1 to 27, wherein the compound of formula (I) or (10b) and sotorasib are provided in jointly therapeutically effective amounts.

29. The method of any one of claims 1 to 27, wherein the compound of formula (I) or (10b) and sotorasib are provided in synergistically effective amounts.

30. The method of any one of claims 1 to 29, wherein the compound of formula (I) or (10b) and/or sotorasib is used at a dose different than when it is used alone.

31. The method of claim 30, wherein the compound of formula (I) or (10b) is used at a dose lower than when it is used alone.

32. The method of claim 30, wherein the compound of formula (I) or (10b) is used at a dose higher than when it is used alone.

33. The method of any one of claims 30 to 32, wherein sotorasib is used at a dose lower than when it is used alone.

34. The method of any one of claims 30 to 32, wherein sotorasib is used at a dose higher than when it is used alone.

35. The method of any one of claims 1 to 34, wherein the treating comprises one or more treatment cycles; each of one or more treatment cycles has a duration of about 28 days; and the compound of formula (I) or (10b) and/or sotorasib are administered daily.

36. The method of any one of claims 1 to 35, wherein the administration of the compound of formula (I) or (10b) and sotorasib comprises one or more dose escalations, dose retentions, or dose de-escalations of the compound of formula (I) or (10b) and/or sotorasib.

37. The method of claim 36, wherein the administration of the compound of formula (I) or (10b) and sotorasib comprises one or more dose escalations, dose retentions, or dose de-escalations of the compound of formula (I) or (10b).

38. The method of claim 37, wherein the one or more dose escalations, dose retentions, or dose de-escalations of the compound of formula (I) or (10b) are determined by a dose-limiting toxicity (DLT) assessment.

39. The method of claim 38, wherein the administration of the compound of formula (I) or (10b) comprises a dose escalation after a previous treatment cycle, when a dose-limiting toxicity (DLT) rate is less than about 19.7% as determined by a DLT assessment.

40. The method of claim 38, wherein the administration of the compound of formula (I) or (10b) comprises a dose de-escalation after a previous treatment cycle, when a dose-limiting toxicity rate is more than about 29.8% as determined by a DLT assessment.

41. The method of claim 38, wherein the administration of the compound of formula (I) or (10b) comprises a dose retention after a previous treatment cycle, when a dose-limiting toxicity rate is in a range of from about 21.9% to about 29.8% as determined by a DLT assessment.

42. The method of any one of claims 35 to 41, wherein the treating comprises a dose escalation period, and wherein, after the dose escalation period, the treating further comprises a dose expansion/optimization period; and the compound of formula (I) or (10b) is administered at a dose regimen determined during the dose escalation period.

43. The method of claim 42, wherein, during the dose expansion/optimization period, the administration of the compound of formula (I) or (10b) comprises one or more dose adjustments.

44. The method of any one of claims 1 to 43, wherein the therapeutically effective amount of sotorasib is a total daily dosage of about 120 mg, about 240 mg, about 360 mg, about 480 mg, about 600 mg, about 720 mg, about 840 mg, or about 960 mg.

45. The method of claim 44, wherein the therapeutically effective amount of sotorasib is a total daily dosage of about 960 mg.

46. The method of any one of claims 1 to 45, wherein the therapeutically effective amount of the compound of formula (I) or (10b) is a total daily dosage of from about 100 mg to about 2000 mg, from about 150 mg to about 1000 mg, from about 200 mg to about 1000 mg, from about 250 mg to about 1000 mg, from about 300 mg to about 1000 mg, from about 350 mg to about 1000 mg, from about 400 mg to about 1000 mg, from about 450 mg to about 1000 mg, from about 500 mg to about 1000 mg, from about 550 mg to about 1000 mg, from about 600 mg to about 1000 mg, from about 650 mg to about 1000 mg, from about 700 mg to about 1000 mg, from about 100 mg to about 700 mg, from about 150 mg to about 700 mg, from about 200 mg to about 700 mg, from about 250 mg to about 700 mg, from about 300 mg to about 700 mg, from about 3 mg to about 700 mg, from about 400 mg to about 700 mg, from about 450 mg to about 700 mg, from about 500 mg to about 700 mg, from about 550 mg to about 700 mg, from about 100 mg to about 550 mg, from about 150 mg to about 550 mg, from about 200 mg to about 550 mg, from about 250 mg to about 550 mg, from about 300 mg to about 550 mg, from about 350 mg to about 550 mg, from about 400 mg to about 550 mg, from about 450 mg to about 550 mg, from about 100 mg to about 400 mg, from about 150 mg to about 400 mg, from about 200 mg to about 400 mg, from about 250 mg to about 400 mg, or from about 300 mg to about 400 mg, on a salt-free and anhydrous basis.

47. The method of claim 46, wherein the therapeutically effective amount is a total daily dosage of from about 250 mg to about 400 mg, from about 400 mg to about 550 mg, or from about 550 mg to about 700 mg of the compound of formula (I) or (10b), on a salt-free and anhydrous basis.

48. The method of claim 47, wherein the therapeutically effective amount is a total daily dosage of about 250 mg, about 400 mg, or about 550 mg of the compound of formula (I) or (10b), on a salt-free and anhydrous basis.

49. The method of any one of claims 46 to 48, wherein the therapeutically effective amount is a total daily dosage of about 250 mg of the compound of formula (I) or (10b), on a salt-free and anhydrous basis.

50. The method of any one of claims 46 to 48, wherein the therapeutically effective amount is a total daily dosage of about 400 mg of the compound of formula (I) or (10b), on a salt-free and anhydrous basis.

51. The method of any one of claims 46 to 48, wherein the therapeutically effective amount is a total daily dosage of about 550 mg of the compound of formula (I) or (10b), on a salt-free and anhydrous basis.

52. The method of any one of claims 1 to 51, wherein the compound of formula (10b) and sotorasib are each administered orally.

53. The method of any one of claims 1 to 52, wherein the compound of formula (10b) is administered once, twice, three times, or four times daily.

54. The method of claim 53, wherein the compound of formula (I) or (10b) is administered once daily; and sotorasib is administered once daily.

55. The method of any one of claims 1 to 54, wherein the compound of formula (I) or (10b) is provided in a tablet formulation.

56. The method of any one of claims 1 to 55, wherein the treating reduces a volume of the cancer or a solid tumor at least about 10%, about 20%, about 30%, about 40%, about 50%, about 60%, about 70%, about 80%, or about 90%.

57. The method of any one of claims 1 to 55, wherein the treating stabilizes the cancer or a solid tumor.

58. The method of any one of claims 1 to 57, wherein the subject is further evaluated for one or more biomarkers that correlate to an antitumor response.

59. A kit for treating cancer in a subject, comprising:
a) a therapeutically effective amount of a compound represented by formula (I): 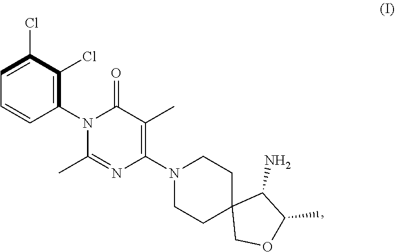
or a pharmaceutically acceptable salt, hydrate, solvate, stereoisomer, conformational isomer, tautomer, or a combination thereof, or a compound represented by formula (10b): 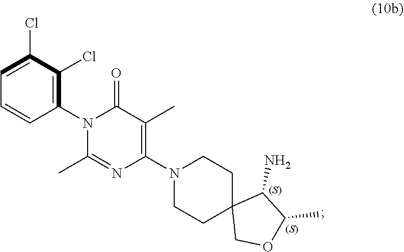
and
b) a therapeutically effective amount of sotorasib,
together with instruction for effective administration.

60. The kit of claim 59, wherein the compound of formula (I) or (10b) and sotorasib are formulated for concomitant administration.

61. The kit of claim 59, wherein the compound of formula (I) or (10b) and sotorasib are formulated for sequential administration.
